SEQUENCE LISTING (43 sequences)

SEQ ID NO: 1 - DNA, 2034 bp, Homo sapiens
atgatgcaagaatctgggactgagacaaaaagtaacggttcagccatccagaatgggtcgggcggcagcaaccacttactagagtgcggcggtcttcgggaggggcggtccaacggagagacgccggccgtggacatcggggcagctgacctcgcccacgcccagcagcagcagcaacaggcacttcaggtggcaagacagctccttcttcagcagcaacagcagcagcaagttagtggattaaaatctcccaagaggaatgacaaacaaccagctcttcaggttcccgtgtcagtggctatgatgacacctcaagttatcactccccagcaaatgcagcagatcctccagcaacaagtgctgagccctcagcagctccaggttctcctccagcagcagcaggccctcatgcttcaacagcagcagcttcaagagttttataaaaaacaacaggaacagttgcagcttcaacttttacaacaacaacatgctggaaaacagcctaaagagcaacagcaggtggctacccagcagttggcttttcagcagcagcttttacagatgcagcagttacagcagcagcacctcctgtctttgcagcgccaaggccttctgacaattcagcccgggcagcctgcccttccccttcaacctcttgctcaaggcatgattccaacagaactgcagcagctctggaaagaagtgacaagtgctcatactgcagaagaaaccacaggcaacaatcacagcagtttggatctgaccacgacatgtgtctcctcctctgcaccttccaagacctccttaataatgaacccacatgcctctaccaatggacagctctcagtccacactcccaaaagggaaagtttgtcccatgaggagcacccccatagccatcctctctatggacatggtgtatgcaagtggccaggctgtgaagcagtgtgcgaagatttccaatcatttctaaaacatctcaacagtgagcatgcgctggacgatagaagtacagcccaatgtagagtacaaatgcaggttgtacagcagttagagctacagcttgcaaaagacaaagaacgcctgcaagccatgatgacccacctgcatgtgaagtctacagaacccaaagccgcccctcagcccttgaatctggtatcaagtgtcactctctccaagtccgcatcggaggcttctccacagagcttacctcatactccaacgaccccaaccgcccccctgactcccgtcacccaaggcccctctgtcatcacaaccaccagcatgcacacggtgggacccatccgcaggcggtactcagacaaatacaacgtgcccatttcgtcagcagatattgcgcagaaccaagaattttataagaacgcagaagttagaccaccatttacatatgcatctttaattaggcaggccattctcgaatctccagaaaagcagctaacactaaatgagatctataactggttcacacgaatgtttgcttacttccgacgcaacgcggccacgtggaagaatgcagtgcgtcataatcttagtcttcacaagtgttttgtgcgagtagaaaacgttaaaggggcagtatggacagtggatgaagtagaattccaaaaacgaaggccacaaaagatcagtggtaacccttcccttattaaaaacatgcagagcagccacgcctactgcacacctctcaatgcagctttacaggcttcaatggctgagaatagtatacctctatacactaccgcttccatgggaaatcccactctgggcaacttagccagcgcaatacgggaagagctgaacggggcaatggagcataccaacagcaacgagagtgacagcagtccaggcagatctcctatgcaagccgtgcatcctgtacacgtcaaagaagagcccctcgatccagaggaagctgaagggcccctgtccttagtgacaacagccaaccacagtccagattttgaccatgacagagattacgaagatgaaccagtaaacgaggacatggagtga

SEQ ID NO: 2 - DNA, 122 bp, Homo sapiens
aatgcagtgcgtcataatcttagtcttcacaagtgttttgtgcgagtagaaaacgttaaaggggcagtatggacagtggatgaagtagaattccaaaaacgaaggccacaaaagatcagtgg

SEQ ID NO: 3 - DNA, 2082 bp, Homo sapiens
atgatgcaagaatctgggactgagacaaaaagtaacggttcagccatccagaatgggtcgggcggcagcaaccacttactagagtgcggcggtcttcgggaggggcggtccaacggagagacgccggccgtggacatcggggcagctgacctcgcccacgcccagcagcagcagcaacaggcacttcaggtggcaagacagctccttcttcagcagcaacagcagcagcaagttagtggattaaaatctcccaagaggaatgacaaacaaccagctcttcaggttcccgtgtcagtggctatgatgacacctcaagttatcactccccagcaaatgcagcagatcctccagcaacaagtgctgagccctcagcagctccaggttctcctccagcagcagcaggccctcatgcttcaacagcagcagcttcaagagttttataaaaaacaacaggaacagttgcagcttcaacttttacaacaacaacatgctggaaaacagcctaaagagcaacagcaggtggctacccagcagttggcttttcagcagcagcttttacagatgcagcagttacagcagcagcacctcctgtctttgcagcgccaaggccttctgacaattcagcccgggcagcctgcccttccccttcaacctcttgctcaaggcatgattccaacagaactgcagcagctctggaaagaagtgacaagtgctcatactgcagaagaaaccacaggcaacaatcacagcagtttggatctgaccacgacatgtgtctcctcctctgcaccttccaagacctccttaataatgaacccacatgcctctaccaatggacagctctcagtccacactcccaaaagggaaagtttgtcccatgaggagcacccccatagccatcctctctatggacatggtgtatgcaagtggccaggctgtgaagcagtgtgcgaagatttccaatcatttctaaaacatctcaacagtgagcatgcgctggacgatagaagtacagcccaatgtagagtacaaatgcaggttgtacagcagttagagctacagcttgcaaaagacaaagaacgcctgcaagccatgatgacccacctgcatgtgaagtctacagaacccaaagccgcccctcagcccttgaatctggtatcaagtgtcactctctccaagtccgcatcggaggcttctccacagagcttacctcatactccaacgaccccaaccgcccccctgactcccgtcacccaaggcccctctgtcatcacaaccaccagcatgcacacggtgggacccatccgcaggcggtactcagacaaatacaacgtgcccatttcgtcagcagatattgcgcagaaccaagaattttataagaacgcagaagttagaccaccatttacatatgcatctttaattaggcaggccattctcgaatctccagaaaagcagctaacactaaatgagatctataactggttcacacgaatgtttgcttacttccgacgcaacgcggccacgtggaagggtgccattcgcaccaacctctcactgcataagtgctttatcagagtagaggatgagtttgggtccttttggacagttgatgatgaagagtttaaacgtggccgccatattcaacgaggccgtcctcgcaaatactgccccgatgaaaactttgacgagcttgtcgcacataacccttcccttattaaaaacatgcagagcagccacgcctactgcacacctctcaatgcagctttacaggcttcaatggctgagaatagtatacctctatacactaccgcttccatgggaaatcccactctgggcaacttagccagcgcaatacgggaagagctgaacggggcaatggagcataccaacagcaacgagagtgacagcagtccaggcagatctcctatgcaagccgtgcatcctgtacacgtcaaagaagagcccctcgatccagaggaagctgaagggcccctgtccttagtgacaacagccaaccacagtccagattttgaccatgacagagattacgaagatgaaccagtaaacgaggacatggagtga

SEQ ID NO: 4 - DNA, 170 bp, Homo sapiens
ggtgccattcgcaccaacctctcactgcataagtgctttatcagagtagaggatgagtttgggtccttttggacagttgatgatgaagagtttaaacgtggccgccatattcaacgaggccgtcctcgcaaatactgccccgatgaaaactttgacgagcttgtcgcaca

SEQ ID NO: 5 - DNA, 2118 bp, Mus musculus
atgatgcaagaatctgggtctgagacaaaaagtaacggatcagccatccagaacgggtccagcggtggcaaccacttactagagtgcggggcacttcgtgacactcggtccaacggagaggcaccagcggtggacctgggggcagccgaccttgcccacgtccagcagcagcaacagcaggccctgcaggtggcaaggcagctcctccttcagcagcagcaacagcagcagcagcaacagcagcagcagcagcagcagcaacagcagcaacagcagcagcaacaacagcagcagcagcaacagcagcagcagcagcagcaagttagtggattaaagtctcccaagaggaatgacaagcaaccagctcttcaggttcccgtgtcagtggctatgatgacacctcaggttatcactcctcaacaaatgcagcagatcctccagcagcaggtgctgagcccccagcagctccaggttctcctccagcagcagcaggccctcatgcttcaacagcagcttcaagaattttataaaaaacaacaggaacagttgcagcttcaacttctccaacagcaacatgctggaaaacagccgaaagagcagcaggtggctacccagcagttggctttccagcagcagcttctccagatgcagcagctgcagcagcagcacctcttgtctctccagcgccaaggcctcctaacaattcagccggggcagcctgcccttcccctccaacccctcgctcaaggcatgattccaacggaactgcagcagctctggaaagaagtgacaagtgcccacactgcagaggaaaccacaagcagcaaccacagcagcctagacctgaccagcacatgtgtctcgtcctcggcaccttccaagtcctccctaatcatgaacccgcatgcctctaccaatggacagctctcggtccacactcccaaaagggaaagcttgtcccacgaggagcacccccacagccaccctctctatggacatggcgtatgcaagtggccaggctgtgaggcggtttgtgacgacttcccagcctttctaaaacatctcaacagtgagcatgcgctggacgatagaagcacagctcaatgtagagtacaaatgcaggttgtacagcagttagagctacagcttgcaaaagacaaagagcgcctgcaagccatgatgacccacctgcatgtgaagtctacagaacccaaagctgcccctcagcccctgaatctggtatcaagtgtcaccctctccaagtctgcctcagaggcttctccacagagcttacctcatactccaacaacccccaccgcccccctgactcctgtcacccaaggcccctccgtcatcaccaccaccagcatgcacacggtgggacctatccgcaggcggtactcagacaaatacaacgtgcccatttcttcagcagatattgcgcagaaccaagaattttataagaacgcggaagttagaccaccatttacatatgcatctttaatcaggcaggccattctcgaatctccagaaaagcagctaacactaaacgaaatctataactggttcacacgaatgtttgcttacttccgacgcaatgcagccacgtggaagaatgcagtgcgtcataatcttagtctccacaagtgttttgtgcgagtagagaacgttaaaggggcagtatggacagtggatgaagtagagttccaaaaacggaggccacaaaagatcagtggtaacccttcccttattaaaaacatgcagagcagccacgcctactgcacacctctcaatgcagctttacaggcttccatggctgagaatagtatacctctgtacactaccgcttccatgggaaatcccactctgggcagcctggccagtgccatccgggaggagctgaacggggccatggagcacaccaacagcaacgagagtgacagcagtccaggcagatcccctatgcaagctgtgcaccccatacacgtcaaagaagaacccctcgaccccgaggaagctgaaggccctctgtccttagtgacaacagccaaccacagtccagattttgaccatgacagagattacgaagacgaaccagtaaatgaggacatggagtga

SEQ ID NO: 6 - DNA, 122 bp, Mus musculus
aatgcagtgcgtcataatcttagtctccacaagtgttttgtgcgagtagagaacgttaaaggggcagtatggacagtggatgaagtagagttccaaaaacggaggccacaaaagatcagtgg

SEQ ID NO: 7 - DNA, 2166 bp, Mus musculus
atgatgcaagaatctgggtctgagacaaaaagtaacggatcagccatccagaacgggtccagcggtggcaaccacttactagagtgcggggcacttcgtgacactcggtccaacggagaggcaccagcggtggacctgggggcagccgaccttgcccacgtccagcagcagcaacagcaggccctgcaggtggcaaggcagctcctccttcagcagcagcaacagcagcagcagcaacagcagcagcagcagcagcagcaacagcagcaacagcagcagcaacaacagcagcagcagcaacagcagcagcagcagcagcaagttagtggattaaagtctcccaagaggaatgacaagcaaccagctcttcaggttcccgtgtcagtggctatgatgacacctcaggttatcactcctcaacaaatgcagcagatcctccagcagcaggtgctgagcccccagcagctccaggttctcctccagcagcagcaggccctcatgcttcaacagcagcttcaagaattttataaaaaacaacaggaacagttgcagcttcaacttctccaacagcaacatgctggaaaacagccgaaagagcagcaggtggctacccagcagttggctttccagcagcagcttctccagatgcagcagctgcagcagcagcacctcttgtctctccagcgccaaggcctcctaacaattcagccggggcagcctgcccttcccctccaacccctcgctcaaggcatgattccaacggaactgcagcagctctggaaagaagtgacaagtgcccacactgcagaggaaaccacaagcagcaaccacagcagcctagacctgaccagcacatgtgtctcgtcctcggcaccttccaagtcctccctaatcatgaacccgcatgcctctaccaatggacagctctcggtccacactcccaaaagggaaagcttgtcccacgaggagcacccccacagccaccctctctatggacatggcgtatgcaagtggccaggctgtgaggcggtttgtgacgacttcccagcctttctaaaacatctcaacagtgagcatgcgctggacgatagaagcacagctcaatgtagagtacaaatgcaggttgtacagcagttagagctacagcttgcaaaagacaaagagcgcctgcaagccatgatgacccacctgcatgtgaagtctacagaacccaaagctgcccctcagcccctgaatctggtatcaagtgtcaccctctccaagtctgcctcagaggcttctccacagagcttacctcatactccaacaacccccaccgcccccctgactcctgtcacccaaggcccctccgtcatcaccaccaccagcatgcacacggtgggacctatccgcaggcggtactcagacaaatacaacgtgcccatttcttcagcagatattgcgcagaaccaagaattttataagaacgcggaagttagaccaccatttacatatgcatctttaatcaggcaggccattctcgaatctccagaaaagcagctaacactaaacgaaatctataactggttcacacgaatgtttgcttacttccgacgcaatgcagccacgtggaagggtgccattcgcaccaacctctcactgcataagtgctttatcagagtagaggatgagtttgggtccttttggacagttgatgatgaagagtttatacgtggccgccatattcaacgaggccgtcctcgcaaatactgccccggtgaaaactctgacgagcttggcgcacataacccttcccttattaaaaacatgcagagcagccacgcctactgcacacctctcaatgcagctttacaggcttccatggctgagaatagtatacctctgtacactaccgcttccatgggaaatcccactctgggcagcctggccagtgccatccgggaggagctgaacggggccatggagcacaccaacagcaacgagagtgacagcagtccaggcagatcccctatgcaagctgtgcaccccatacacgtcaaagaagaacccctcgaccccgaggaagctgaaggccctctgtccttagtgacaacagccaaccacagtccagattttgaccatgacagagattacgaagacgaaccagtaaatgaggacatggagtga

SEQ ID NO: 8 - DNA, 170 bp, Mus musculus
ggtgccattcgcaccaacctctcactgcataagtgctttatcagagtagaggatgagtttgggtccttttggacagttgatgatgaagagtttatacgtggccgccatattcaacgaggccgtcctcgcaaatactgccccggtgaaaactctgacgagcttggcgcaca

SEQ ID NO: 9 - protein, 677 aa, Homo sapiens
MMQESGTETKSNGSAIQNGSGGSNHLLECGGLREGRSNGETPAVDIGAADLAHAQQQQQQALQVARQLLLQQQQQQQVSGLKSPKRNDKQPALQVPVSVAMMTPQVITPQQMQQILQQQVLSPQQLQVLLQQQQALMLQQQQLQEFYKKQQEQLQLQLLQQQHAGKQPKEQQQVATQQLAFQQQLLQMQQLQQQHLLSLQRQGLLTIQPGQPALPLQPLAQGMIPTELQQLWKEVTSAHTAEETTGNNHSSLDLTTTCVSSSAPSKTSLIMNPHASTNGQLSVHTPKRESLSHEEHPHSHPLYGHGVCKWPGCEAVCEDFQSFLKHLNSEHALDDRSTAQCRVQMQVVQQLELQLAKDKERLQAMMTHLHVKSTEPKAAPQPLNLVSSVTLSKSASEASPQSLPHTPTTPTAPLTPVTQGPSVITTTSMHTVGPIRRRYSDKYNVPISSADIAQNQEFYKNAEVRPPFTYASLIRQAILESPEKQLTLNEIYNWFTRMFAYFRRNAATWKNAVRHNLSLHKCFVRVENVKGAVWTVDEVEFQKRRPQKISGNPSLIKNMQSSHAYCTPLNAALQASMAENSIPLYTTASMGNPTLGNLASAIREELNGAMEHTNSNESDSSPGRSPMQAVHPVHVKEEPLDPEEAEGPLSLVTTANHSPDFDHDRDYEDEPVNEDME

SEQ ID NO: 10 - protein, 40 aa, Homo sapiens
NAVRHNLSLHKCFVRVENVKGAVWTVDEVEFQKRRPQKIS

SEQ ID NO: 11 - protein, 693 aa, Homo sapiens
MMQESGTETKSNGSAIQNGSGGSNHLLECGGLREGRSNGETPAVDIGAADLAHAQQQQQQALQVARQLLLQQQQQQQVSGLKSPKRNDKQPALQVPVSVAMMTPQVITPQQMQQILQQQVLSPQQLQVLLQQQQALMLQQQQLQEFYKKQQEQLQLQLLQQQHAGKQPKEQQQVATQQLAFQQQLLQMQQLQQQHLLSLQRQGLLTIQPGQPALPLQPLAQGMIPTELQQLWKEVTSAHTAEETTGNNHSSLDLTTTCVSSSAPSKTSLIMNPHASTNGQLSVHTPKRESLSHEEHPHSHPLYGHGVCKWPGCEAVCEDFQSFLKHLNSEHALDDRSTAQCRVQMQVVQQLELQLAKDKERLQAMMTHLHVKSTEPKAAPQPLNLVSSVTLSKSASEASPQSLPHTPTTPTAPLTPVTQGPSVITTTSMHTVGPIRRRYSDKYNVPISSADIAQNQEFYKNAEVRPPFTYASLIRQAILESPEKQLTLNEIYNWFTRMFAYFRRNAATWKGAIRTNLSLHKCFIRVEDEFGSFWTVDDEEFKRGRHIQRGRPRKYCPDENFDELVAHNPSLIKNMQSSHAYCTPLNAALQASMAENSIPLYTTASMGNPTLGNLASAIREELNGAMEHTNSNESDSSPGRSPMQAVHPVHVKEEPLDPEEAEGPLSLVTTANHSPDFDHDRDYEDEPVNEDME

SEQ ID NO: 12 - protein, 56 aa, Homo sapiens
GAIRTNLSLHKCFIRVEDEFGSFWTVDDEEFKRGRHIQRGRPRKYCPDENFDELVA

SEQ ID NO: 13 - protein, 705 aa, Mus musculus
MMQESGSETKSNGSAIQNGSSGGNHLLECGALRDTRSNGEAPAVDLGAADLAHVQQQQQQALQVARQLLLQQQQQQQQQQQQQQQQQQQQQQQQQQQQQQQQQQQQQVSGLKSPKRNDKQPALQVPVSVAMMTPQVITPQQMQQILQQQVLSPQQLQVLLQQQQALMLQQQLQEFYKKQQEQLQLQLLQQQHAGKQPKEQQVATQQLAFQQQLLQMQQLQQQHLLSLQRQGLLTIQPGQPALPLQPLAQGMIPTELQQLWKEVTSAHTAEETTSSNHSSLDLTSTCVSSSAPSKSSLIMNPHASTNGQLSVHTPKRESLSHEEHPHSHPLYGHGVCKWPGCEAVCDDFPAFLKHLNSEHALDDRSTAQCRVQMQVVQQLELQLAKDKERLQAMMTHLHVKSTEPKAAPQPLNLVSSVTLSKSASEASPQSLPHTPTTPTAPLTPVTQGPSVITTTSMHTVGPIRRRYSDKYNVPISSADIAQNQEFYKNAEVRPPFTYASLIRQAILESPEKQLTLNEIYNWFTRMFAYFRRNAATWKNAVRHNLSLHKCFVRVENVKGAVWTVDEVEFQKRRPQKISGNPSLIKNMQSSHAYCTPLNAALQASMAENSIPLYTTASMGNPTLGSLASAIREELNGAMEHTNSNESDSSPGRSPMQAVHPIHVKEEPLDPEEAEGPLSLVTTANHSPDFDHDRDYEDEPVNEDME

SEQ ID NO: 14 - protein, 40 aa, Mus musculus; identical to SEQ ID NO: 10

SEQ ID NO: 15 - protein, 721 aa, Mus musculus
MMQESGSETKSNGSAIQNGSSGGNHLLECGALRDTRSNGEAPAVDLGAADLAHVQQQQQQALQVARQLLLQQQQQQQQQQQQQQQQQQQQQQQQQQQQQQQQQQQQQVSGLKSPKRNDKQPALQVPVSVAMMTPQVITPQQMQQILQQQVLSPQQLQVLLQQQQALMLQQQLQEFYKKQQEQLQLQLLQQQHAGKQPKEQQVATQQLAFQQQLLQMQQLQQQHLLSLQRQGLLTIQPGQPALPLQPLAQGMIPTELQQLWKEVTSAHTAEETTSSNHSSLDLTSTCVSSSAPSKSSLIMNPHASTNGQLSVHTPKRESLSHEEHPHSHPLYGHGVCKWPGCEAVCDDFPAFLKHLNSEHALDDRSTAQCRVQMQVVQQLELQLAKDKERLQAMMTHLHVKSTEPKAAPQPLNLVSSVTLSKSASEASPQSLPHTPTTPTAPLTPVTQGPSVITTTSMHTVGPIRRRYSDKYNVPISSADIAQNQEFYKNAEVRPPFTYASLIRQAILESPEKQLTLNEIYNWFTRMFAYFRRNAATWKGAIRTNLSLHKCFIRVEDEFGSFWTVDDEEFIRGRHIQRGRPRKYCPGENSDELGAHNPSLIKNMQSSHAYCTPLNAALQASMAENSIPLYTTASMGNPTLGSLASAIREELNGAMEHTNSNESDSSPGRSPMQAVHPIHVKEEPLDPEEAEGPLSLVTTANHSPDFDHDRDYEDEPVNEDME

SEQ ID NO: 16 - protein, 56 aa, Mus musculus
GAIRTNLSLHKCFIRVEDEFGSFWTVDDEEFIRGRHIQRGRPRKYCPGENSDELGA

SEQ ID NO: 17 - DNA, 19 bp, Mus musculus
cagtggatgaagtagagtt

SEQ ID NO: 18 - DNA, 19 bp, Mus musculus
gatgaagtagagttccaaa

SEQ ID NO: 19 - DNA, 19 bp, Mus musculus
gtggtaacccttcccttat

SEQ ID NO: 20 - DNA, 19 bp, Mus musculus
tggtaacccttcccttatt

SEQ ID NO: 21 - DNA, 19 bp, Mus musculus
ctttatcagagtagaggat

SEQ ID NO: 22 - DNA, 19 bp, Mus musculus
cagttgatgatgaagagtt

SEQ ID NO: 23 - DNA, 19 bp, Mus musculus
acaaaagatcagtggtaac

SEQ ID NO: 24 - DNA, 19 bp, Mus musculus
gtggtaacccttcccttat

SEQ ID NO: 25 - DNA, 19 bp, Homo sapiens
cagtggatgaagtagaatt

SEQ ID NO: 26 - DNA, 19 bp, Homo sapiens
ccacgtggaagaatgcagt

SEQ ID NO: 27 - DNA, 19 bp, Homo sapiens
agtggtaacccttccctta

SEQ ID NO: 28 - DNA, 19 bp, Homo sapiens
aaagatcagtggtaaccct

SEQ ID NO: 29 - DNA, 19 bp, Homo sapiens
ccatattcaacgaggccgt

SEQ ID NO: 30 - DNA, 19 bp, Homo sapiens
ccaacctctcactgcataa

SEQ ID NO: 31 - DNA, 19 bp, Homo sapiens
gcacataacccttccctta

SEQ ID NO: 32 - DNA, 19 bp, Homo sapiens
acgagcttgtcgcacataa

SEQ ID NO: 33 - DNA, 22 bp, Mus musculus
gcaagctgaccctgaagttcat

SEQ ID NO: 34 - DNA, 21 bp, Mus musculus
gacagtggatgaagtagagtt

SEQ ID NO: 35 - DNA, 21 bp, Mus musculus
gacgagcttggcgcacataac

SEQ ID NO: 36 - DNA, 300 bp, Homo sapiens
ttttcagatttgtactctggaacctattttgggcttgtatgcatattaaaaaatatttctgctggtcagttattaccagagactagacgaaaatgccaaagtacgattaatttataattttggtttatttttgcttttggaaaagacatccttaaccatgagaaggtcctccacgggggtggtagctacagctcttggggatggttttacaaatgtggactgttaggctgaatttactgctgtttttggagtcttttcttatagaaaatgtaaatgttctctcttttatacttgctgcag

SEQ ID NO: 37 - DNA, 300 bp, Homo sapiens
gtacgtccaccatgtttgaacttcttagcctagcttggattgtgctaacaagtataacgtagaggctttggctgctagatggtgttagaccaaagaactggtttcacgttgtctcagttaagggaagcaaaaaagcatttcatccctcctgttttgtatacccaccagtccccctgccctgcttgctggtctttgttgcatcacatgctagtagcttttaggtggcaatgcatattaaccctcacaaaccatttgcttatgcttattgcattttattttctttttcctgttccctgtatt

SEQ ID NO: 38 - DNA, 300 bp, Homo sapiens
ctagatggtgttagaccaaagaactggtttcacgttgtctcagttaagggaagcaaaaaagcatttcatccctcctgttttgtatacccaccagtccccctgccctgcttgctggtctttgttgcatcacatgctagtagcttttaggtggcaatgcatattaaccctcacaaaccatttgcttatgcttattgcattttattttctttttcctgttccctgtattggatgtctgttctgcccccaaccccgtctccttttgttgccactccatctcctttgctgctgctcctatcccag

SEQ ID NO: 39 - DNA, 300 bp, Homo sapiens
gtaagagcctttacttgcttttgttttttttttctgctgttgctttggctttgcatggttgactcatcccatgtagttttgcatttcaccaatagcagtgccacagctttctgcaacgaaactaaattgtgtgatttgtacatgtattgctctcgtttctctttggaacaccagtgccttgaataatgcttttttgacgtctttaatgcaaatattcgttctattttgattcagcagcttacggggaccctagaactaaatacttgtgcttaacccataatcccccctctctccccaccc

SEQ ID NO: 40 - DNA, 300 bp, Mus musculus
ttatgtggagggacctgcttgggacttgcatgcatgtttaccagtgtgtcttctgatcacatggtaccagaaattaggcttaaatgccagagtaccatccattcataatctgcggtttttcttaaaagacactcggaagaagatcctccgtgggggtgggggtgggggtggcggctacggctctggaggtcggttttacacatgtggggctgaatgtactgctgtgtgtgttttttttttttttgagtcttttcttatagaaaaaaaatgtcaatgttctctcttttatacttgctgcag

SEQ ID NO: 41 - DNA, 300 bp, Mus musculus
gtacgtccgccatgtttgaacttcttagcccagctcggattgtgctaacgagtgtactgtagaggctgctaggcggtgtcagaccaaagacctggttttcacgtttgtctcagttaagggaagcaagagaacatttcatcccttctgtctcgtatacccaccagtccccctgcccctgcttgctggtctctgttacatcccatgcgagtagcttttaggcggcgttgcatattaaccctcacgagccattcgcttatgcttattgcattttattttctttttcctgttccctgtattgga

SEQ ID NO: 42 - DNA, 300 bp, Mus musculus
ggcggtgtcagaccaaagacctggttttcacgtttgtctcagttaagggaagcaagagaacatttcatcccttctgtctcgtatacccaccagtccccctgcccctgcttgctggtctctgttacatcccatgcgagtagcttttaggcggcgttgcatattaaccctcacgagccattcgcttatgcttattgcattttattttctttttcctgttccctgtattggatgtctgtcctgcccccaaccccgcctccttttgttgccactccatctcctttgctgctgctcctatcccag

SEQ ID NO: 43 - DNA, 300 bp, Mus musculus
gtaagagcctttacttgctttgttctcttgtgtctgctgttgctttggctttgcatggttgactcatcccatgtagtatgacatttcacccaagagcagtgccacagctttctgcaacagaaactaaacagtgtgaactgtacacgcacctcgctcgtttccctttggaacgccagtgcctagaagatgctttttctgtgtctctaatgccgatgatgactgactgtattttgattcagcagctaatggggaccctcaaagctagatatcatcccccatgttacccataatccctgcaag

(19) United States
(12) Patent Application Publication
(10) Pub. No.: US 2014/0134737 A1
(43) Pub. Date: May 15, 2014

(54) Alternative Splicing Modulators and Splice Variants and Their Use in the Control and Detection of Pluripotency and Differentiation

(71) Applicants: Benjamin J. Blencowe, Toronto (CA); Mathieu Gabut, Polliat (FR); Hong Han, Toronto (CA)

(72) Inventors: Benjamin J. Blencowe, Toronto (CA); Mathieu Gabut, Polliat (FR); Hong Han, Toronto (CA)

(73) Assignee: Governing Council of the University of Toronto, Toronto, ON (CA)

(21) Appl. No.: 14/128,052

(22) PCT Filed: Jun. 29, 2012
(86) PCT No.: PCT/CA2012/000616

§ 371 Date: Dec. 20, 2013

Related U.S. Application Data
(60) Provisional application No. 61/503,206, filed on Jun. 30, 2011.

(51) Int. Cl. C12N 5/074

(52) U.S. Cl. CPC C12N 5/0607
USPC 435/455; 435/377

(57) ABSTRACT

Nucleotide sequences encoding novel splice variants of FOXP1, proteins encoded by the novel splice variants and antibodies thereto are disclosed. In addition, methods are described for maintaining a population of homogenous self-renewing and pluripotent stem cells, suppressing stem cell differentiation, and reprogramming somatic cells into pluripotent stem cells comprising the use of the novel splice variants. Also disclosed are modulators of alternative splicing such as MBNL1 and MBNL2 and methods and uses thereof for promoting pluripotency.

### CROSS REFERENCE TO RELATED APPLICATIONS

This non-provisional application claims priority from U.S. provisional application No. 61/503,206 filed on Jun. 30, 2011, which is incorporated herein by reference in its entirety.

### FIELD OF THE DISCLOSURE

The disclosure relates to a novel splice variant of FOXP1 and methods and uses thereof. In particular, the disclosure relates to methods of reprogramming somatic cells into pluripotent stem cells and methods of maintaining pluripotent stem cells through the use of the novel splice variant. The disclosure also relates to modulators of alternative splicing for promoting pluripotency and methods and uses thereof.

### BACKGROUND OF THE DISCLOSURE

During the past several years great strides have been made in understanding the regulatory processes that are responsible for maintenance of the pluripotent state of embryonic stem cells (ESCs), and for the reprogramming of somatic cells to generate ESCs and induced pluripotent stem cells (iPSCs). A core set of transcription factors that includes Oct4, Nanog, Sox2 and Tcf3 functions in ESC maintenance. The first three of these factors induce and cross regulate each other's expression, and also activate genes that further stabilize the ESC state (Chen et al., 2008; Kim et al., 2008; Silva et al., 2009). In a landmark discovery it was demonstrated that Oct4 and Sox2, together with Klf4 and c-Myc can reprogram somatic cells to iPSCs (Takahashi and Yamanaka, 2006). These factors remodel the transcriptome through successive reprogramming stages (Samavarchi-Tehrani et al., 2010) that ultimately lead to the reactivation of endogenous Oct4, Nanog and Sox2 and the establishment of the core transcriptional regulatory network required to maintain pluripotency.

In contrast to the major progress in the elucidation of signaling pathways and transcription factor networks in the control of pluripotency and iPSC formation, the role of alternative splicing (AS) in this process is not well understood. Likewise, while transcripts from nearly all human multi-exon genes undergo AS (Pan et al., 2008; Wang et al., 2008), the functional significance of the vast majority of these events is not known. A small number of AS events have been identified that play pivotal roles in the regulation of cell differentiation and early development. Classic examples include AS events in the Drosophila transcription factor genes doublesex and fruitless, which control transcriptional programs required for sex determination and courtship behavior, respectively (Demir and Dickson, 2005; Forch and Valcarcel, 2003). Recent studies comparing splice isoforms between ES and differentiated cell populations have identified AS differences between these two states (Atlasi et al., 2008; Kunarso et al., 2008; Pritsker et al., 2005; Rao et al., 2010b; Salomonis et al., 2010; Wu et al., 2010; Yeo et al., 2007), and two such events have been implicated in changing the activities of Tcf3 and Sall4, transcription factors which function in pluripotency (Rao et al., 2010b; Salomonis et al., 2010). It is therefore emerging that specific AS events may function to modulate transcriptional networks involved in transitions between pluripotency maintenance and cell type specification.

Forkhead box (FOX) proteins belong to a family of metazoan transcription factors that play essential roles in the regulation of genes involved in cell proliferation, differentiation and development, particularly during embryogenesis (Wijchers et al., 2006). The forkhead box, a domain of 80 to 100 amino acids that adopts a winged helix conformation, is a defining feature of FOX proteins and is responsible for binding to DNA (Li et al., 2004). FOXP1 is one of four members of the FOXP subfamily of FOX proteins that contain a C-terminal forkhead motif and N-terminal zinc finger and leucine zipper domains. FOXP1 is widely expressed across human tissues and loss of its expression, or its fusion with other proteins through chromosomal translocations, has been linked to several types of cancer (Koon et al., 2007). Knockout of murine Foxp1 results in early embryonic lethality (Wang et al., 2004) and disruption of Foxp1 expression in adult cells and tissues revealed that it has numerous critical roles in development and the establishment of specific cell types (Dasen et al., 2008; Zhang et al., 2010). Several splice variants of FOXP1 have been identified (Brown et al., 2008), yet the function of these are not well understood.

### SUMMARY OF THE DISCLOSURE

The present inventors have found that an embryonic stem cell (ESC)-specific isoform (FOXP1-ES) of the forkhead family transcription factor FOXP1 promotes the maintenance of ESC pluripotency and the reprogramming of somatic cells to ESCs and induced pluripotent stem cells. In particular, the present inventors identified a highly conserved alternative splicing event in FOXP1 transcripts that is activated in ESCs and silenced during cell differentiation. This alternative splicing event modifies critical amino acid residues within the FOXP1 forkhead domain and alters its DNA binding specificity. In ESCs this alternative splicing event switches the transcriptional regulatory output of FOXP1, which results in the stimulation of pluripotency genes such as OCT4, NANOG, GDF3 and NR5A2 while repressing cell lineage specification and differentiation genes. Induced expression of the ESC-specific isoform of FOXP1 promotes self-renewal and the maintenance of pluripotency, whereas silencing this isoform inhibits efficient iPSC reprogramming.

Accordingly, one aspect of the present disclosure is related to an isolated nucleic acid molecule comprising:

a. a nucleic acid sequence as shown in SEQ ID NOS: 3, 4, 7 or 8;

b. a nucleic acid sequence that is complementary to a nucleic acid sequence of (a);

c. a nucleic acid sequence that has substantial sequence identity to a nucleic acid sequence of (a) or (b);

d. a nucleic acid sequence that hybridizes to a nucleic acid sequence of (a), (b) or (c) under stringent hybridization conditions; or

e. a nucleic acid sequence differing from any of the nucleic acid sequences of (a) to (d) in codon sequences due to the degeneracy of the genetic code.

The disclosure is also related to an isolated nucleic acid molecule encoding an amino acid sequence as shown in SEQ ID NOS: 11, 12, 15 or 16.

The disclosure is further related to an isolated nucleic acid molecule comprising an antisense oligonucleotide to the nucleic acid sequence as shown in any one of SEQ ID NOS: 36 to 43. Optionally, the antisense nucleotide is 2 to 50, 5 to 40 or 10 to 25 nucleotides in length.

The disclosure also provides a recombinant expression vector comprising any of the isolated nucleic acid molecules described above.

In another aspect, the disclosure relates to an isolated polypeptide comprising the amino acid sequence as shown in SEQ ID NOS: 11, 15 or a fragment thereof. Optionally, the fragment comprises amino acids 511 to 565 of SEQ ID NO: 11 or amino acids 538 to 594 of SEQ ID NO: 15.

The disclosure further relates to a binding protein that binds to an isolated polypeptide comprising the amino acid sequence as shown in SEQ ID NOS: 11, 12, 15 or 16 or a fragment thereof. In one embodiment, the binding protein is an antibody, antibody fragment, peptide aptamer or nucleic-acid derived aptamer.

The disclosure also relates to a host cell comprising any of the isolated nucleic acid molecules, recombinant expression vectors, isolated polypeptides, or binding proteins described above.

In another aspect, the disclosure relates to the use of the isolated nucleic acids, vectors, or isolated polypeptides described above, or an antisense or interfering RNA molecule that increases the expression of FOXP1-ES and/or decreases the expression of FOXP1 to produce pluripotent stem cells, to maintain or enhance a homogeneous population of pluripotent stem cells, suppress stem cell differentiation or reprogram somatic cells into pluripotent stem cells.

The disclosure further relates to the use of a cDNA encoding FOXP1, a FOXP1 protein, an antisense or interfering RNA molecule that decreases the expression of FOXP1-ES and/or increases the expression of FOXP1, or a binding protein as described above to produce a population of differentiated cells.

The disclosure also provides a method of reprogramming somatic cells into pluripotent stem cells comprising:
(1)(a) transfecting somatic cells with a cDNA encoding FOXP1-ES,
(b) transfecting somatic cells with a mRNA encoding FOXP1-ES,
(c) expressing cDNA encoding FOXP1-ES in somatic cells,
(d) administering FOXP1-ES protein to a culture of somatic cells,
(e) administering an exon 18b or exon 16b splicing modulator to cells,
(f) administering antisense RNA or interfering RNA that decreases the expression of FOXP1 to cells, or
(g) administering genomic derived FOXP1 to cells and expressing the FOXP1-ES isoform in the cells; and
(2) culturing under conditions that allow reprogramming of the somatic cells into induced pluripotent stem cells.

In another embodiment, the disclosure provide a method of maintaining a homogenous population of pluripotent stem cells comprising:
(1)(a) transfecting cells with a cDNA encoding FOXP1-ES,
(b) transfecting cells with a mRNA encoding FOXP1-ES,
(c) administering FOXP1-ES protein to cells,
(d) expressing cDNA encoding FOXP1-ES in cells,
(e) administering an exon 18b or exon 16b splicing modulator to cells,
(f) administering antisense RNA or interfering RNA that decreases the expression of FOXP1 to cells, or
(g) administering genomic derived FOXP1 to cells and expressing the FOXP1-ES isoform in the cells; and
(2) culturing the cells.

In another embodiment, the disclosure provides a method of suppressing stem cell differentiation comprising:
(1)(a) transfecting cells with a cDNA encoding FOXP1-ES,
(b) transfecting cells with a mRNA encoding FOXP1-ES,
(c) administering FOXP1-ES protein to cells,
(d) expressing cDNA encoding FOXP1-ES in cells,
(e) administering an exon 18b or exon 16b splicing modulator to cells,
(f) administering antisense RNA or interfering RNA that decreases the expression of FOXP1 to cells, or
(g) administering genomic derived FOXP1 to cells and expressing the FOXP1-ES isoform in the cells; and
(2) culturing the cells.

In yet another embodiment, the disclosure provides a method of producing a population of differentiated cells comprising:
(1)(a) transfecting stem cells with a cDNA encoding FOXP1,
(b) transfecting stem cells with a mDNA encoding FOXP1,
(c) administering FOXP1 protein to stem cells,
(d) inhibiting the expression of FOXP1-ES in stem cells,
(e) administering an exon 18b or exon 16b splicing modulator to cells,
(f) administering antisense RNA or interfering RNA that decreases the expression of FOXP1-ES to cells, or
(g) administering genomic derived FOXP1 to cells and expressing the FOXP1 isoform in the cells; and
(2) culturing the cells.

The inventors have also shown that specific splicing regulators including MBNL1, MBNL2, TIA1 and TIAL1 function to modulate the ESC-specific splicing event in FOXP1 transcripts and expression of FOXP1-ES. The regulators act through conserved binding sites for these factors in the intronic regions proximal to this splicing event.

Accordingly, another aspect of the present disclosure is directed to a method of modulating the expression of FOX1P-ES in a cell comprising administering an exon 18b or exon 16b modulator to the cell.

In one embodiment, the modulator is a stimulator of exon 18b inclusion. Optionally, the stimulator of exon 18b inclusion is selected from the group consisting of: TIA1, TIAL1, a MBNL1 antagonist, a MBNL2 antagonist and antisense RNA or small interfering RNA that decreases expression of FOXP1.

In another embodiment, the modulator is a stimulator of exon 16b inclusion. Optionally, the stimulator of exon 16b inclusion is selected from the group consisting of: Tia1, Tial1, a Mbnl1 antagonist, a Mbnl2 antagonist and antisense RNA or small interfering RNA that decreases expression of Foxp1.

In a further embodiment, the MBNL1 antagonist and/or MBNL2 antagonist is an antibody to MBNL1 and/or MBNL2 or peptide or nucleic-acid derived aptamer to MBNL1 and/or MBNL2, antisense RNA or small interfering RNA that decreases expression of MBNL1 and/or MBNL2, or a compound that inhibits the expression or function of MBNL1 and/or MBNL2.

In another embodiment, the modulator is a repressor of exon 18b inclusion. Optionally, the repressor of exon 18b inclusion is selected from the group consisting of: MBNL1, MBNL2, a TIA1 antagonist, a TIAL1 antagonist, and antisense RNA or small interfering RNA that decreases expression of FOXP1-ES.

In another embodiment, the modulator is a repressor of exon 16b inclusion. Optionally, the repressor of exon 16b inclusion is selected from the group consisting of: Mbnl1, Mbnl2, a Tia1 antagonist, a Tial1 antagonist, and antisense RNA or small interfering RNA that decreases expression of Foxp1-ES.

In a further embodiment, the TIA1 antagonist or TIAL2 antagonist is an antibody or peptide or nucleic-acid derived aptamer to TIA1 and/or TIAL1, antisense RNA or small interfering RNA that decreases expression of TIA1 and/or TIAL1, or compound that inhibits the expression or function of TIA1 and/or TIAL1.

The inventors have also found that MBNL1 and MBNL2 are regulators of ESC-specific alternative splicing and reprogramming that act through a number of target genes, including, but not limited to, FOXP1.

Accordingly, the present disclosure also relates to the use of antagonists to MBNL1 and/or MBNL2 to maintain or enhance pluripotency of a cell.

In one embodiment, the disclosure relates to the use of both a MBNL1 antagonist and a MBNL2 antagonist to maintain or enhance pluripotency of a cell.

In one embodiment, maintaining or enhancing pluripotency comprises producing pluripotent stem cells, maintaining a homogeneous population of pluripotent stem cells, suppressing stem cell differentiation or reprogramming somatic cells into pluripotent stem cells.

In another embodiment, maintaining or enhancing pluripotency comprises producing pluripotent stem cells comprises increasing the efficiency and/or kinetics of: producing pluripotent stem cells, maintaining a homogeneous population of pluripotent stem cells, suppressing stem cell differentiation or reprogramming somatic cells into pluripotent stem cells.

The disclosure further relates to a method of assessing the pluripotency of a cell population comprising detecting the level of expression of FOXP1-ES in a sample of cells from the population, wherein an increase in the level of FOXP1-ES compared to a reference level in the sample of cells indicates the pluripotency of the cell population.

The disclosure also relates to a method of assessing the pluripotency of a cell population over time comprising
(a) detecting the level of expression of FOXP1-ES in a sample of cells from the population at a first time point,
(b) detecting the level of expression of FOXP1-ES in a sample of cells from the population at a second time point,
wherein an increase in the level of FOXP1-ES in the sample of cells at the second time point compared to the first time point indicates increased pluripotency of the cell population.

The disclosure further relates to a method of assessing the pluripotency of a cell population comprising detecting the level of expression of MBNL1 and/or MBNL2 in a sample of cells from the population, wherein a decrease in the level of MBNL1 and/or MBNL2 compared to a reference level in the sample of cells indicates the pluripotency of the cell population.

The disclosure also relates to a method of assessing the pluripotency of a cell population over time comprising
(a) detecting the level of expression of MBNL1 and/or MBNL2 in a sample of cells from the population at a first time point,
(b) detecting the level of expression of MBNL1 and/or MBNL2 in a sample of cells from the population at a second time point,
wherein an decrease in the level of MBNL1 and/or MBNL2 in the sample of cells at the second time point compared to the first time point indicates increased pluripotency of the cell population.

Other features and advantages of the present disclosure will become apparent from the following detailed description. It should be understood, however, that the detailed description and the specific examples while indicating embodiments of the disclosure are given by way of illustration only, since various changes and modifications within the spirit and scope of the disclosure will become apparent to those skilled in the art from this detailed description.

### BRIEF DESCRIPTION OF THE DRAWINGS

The disclosure will now be described in relation to the drawings in which:

FIG. 1 shows the identification of an embryonic stem cell (ESC)-specific splice variant from the human and mouse FOXP1/Foxp1 genes.

FIG. 1(A) is a schematic representation of exons 16 to 21 of the human FOXP1 gene. Transcripts including alternative exon 18 (black, [▪]“FOXP1”; NM_{—}032682) encode the widely expressed, canonical form of FOXP1 and transcripts including alternative exon 18b (gray; [symbol] “FOXP1-ES”) are specifically detected in hESCs. Transcripts simultaneously including exons 18 and 18b (indicated by an asterisk) are detected at low levels in hESCs, and are predicted to be targeted by nonsense mediated mRNA decay.

FIG. 1(B) shows RT-PCR assays using primers annealing to FOXP1 exons 17 and 19 (arrows) which were used to analyze FOXP1 splice isoform levels in self-renewing H9 hESCs grown in the presence of MEF feeder cells (lane 1) or matrigel (lane 2), H9 hESCs induced to differentiate for 2 days towards primitive endoderm (lane 3), primitive mesoderm (lane 4), neural lineages (lane 5) and neural progenitor cells (NPCs) at day 10 (lane 6) post induction. FOXP1 splice isoforms were also analyzed in a second hESC line (CA1, lane 7) and in 8 human immortalized cell lines of diverse origin as indicated in lanes 8-15. HeLa, cervical carcinoma; IMR32, neuroblastoma; H538, lung carcinoma; A549, lung adenocarcinoma; Colo 205, colorectal carcinoma; Raji, B lymphblastoma; Jurkat, T lymphoblastoma; 293T; “embryonic kidney”. *, isoform containing both exons 18 and 18b. ACTB mRNA levels are shown for comparison.

FIG. 1(C) shows RT-PCR analysis (as performed in panel B) of FOXP1 splice isoform levels in unsorted H9 hESCs (lane 1) and, following FACS, H9 hESCs that are double negative (lane 2) or double positive (lane 3) for the cell surface expressed pluripotency markers TRA1-81 and SSEA-1. *, isoform containing both exons 18 and 18b. ACTB mRNA levels are shown for comparison.

FIG. 1(D) is a conservation analysis of sequences surrounding FOXP1 human exons 18 and 18b (orthologous to exons 16 and 16b in mouse Foxp1) across 46 vertebrate species. The conservation plot was generated from the UCSC browser using the hg19 genome assembly.

FIG. 1(E) shows a RT-PCR analysis of Foxp1 splice isoforms in self-renewing CGR8 and Hb9 mouse (m)ESC lines (lanes 1 and 7), in CGR8 mESCs differentiated towards neural and glial progenitors (lane 2), in CGR8 mESCs aggregated to form embryoid bodies (EB) grown in conditions favoring differentiation into cardiomyocytes (EB days 2-10, lanes 3-5), and in beating cardiomyocytes (EB day 14, lane 6). Hb9 mESCs were differentiated into motor neuron (MN) progenitors (lane 8) and into mature MNs, which were FACS sorted (lane 9). Analysis of Neuro2a cells is shown in lane 10. *, isoform containing both exons 16 and 16b. Gapdh mRNA levels are shown for comparison.

FIG. 2 shows that FOXP1-ES has a distinct DNA binding specificity compared to the canonical form of FOXP1.

FIG. 2(A) is a multiple alignment of amino acid sequences encoding the FOXP1 and FOXP1-ES forkhead DNA binding domains from different vertebrate species. Amino acid sequences conserved across all species analyzed are indicated in white. Amino acid changes introduced as a consequence of splicing of exon 18b in FOXP1-ES are highlighted in dark grey. Amino acids predicted to contact DNA (based on the co-crystal structure of FOXP2 bound to its recognition site (Stroud et al., 2006)) are indicated in light grey, and residues that are the most highly conserved across Forkhead protein family members are indicated by black arrow heads above the alignment.

FIG. 2(B) is a protein binding microarray (PBM) analysis of the DNA binding preferences of GST fusion proteins containing the forkhead domains of FOXP1 and FOXP1-ES. Relative binding affinities measured as anti-GST florescence signal intensity are represented as “E scores” (Berger et al., 2006). The scatterplot directly compares the E scores for GST-FOXP1 and GST-FOXP1-ES, after averaging data from two independent experiments. Sequences of probes with E scores >0.45 in at least one of the two repeat experiments were clustered to derive consensus binding sites. All probe sequences (regardless of E score) that contain the consensus sequence GTAAACA preferentially bound by GST-FOXP1 are indicated by black dots. All probe sequences that contain the consensus sequences CGATACA, CAATACA or TGATACA and are indicated by dark grey dots with a black line. Additional probe sequences containing C/A-rich motifs that are preferentially bound by FOXP1-ES or similarly by both isoforms are indicated by dark grey and white dots, respectively. Light grey dots indicate all other probe sequences with E scores <0.45. Sequence logos representing PBM-derived consensus binding sites were generated using enoLOGOS. A full version of the scatterplot is shown in FIG. 11A.

FIG. 2(C) is an Electrophoresis Mobility Shift Assay (EMSA) validating PBM-derived consensus DNA binding sites for FOXP1 and FOXP1-ES. Radiolabeled dsDNA probes containing two copies of GTAAACAA (top left panel), AATAAACA (top middle panel) or CGATACAA (top right panel), or two copies of mutant versions of these sequences GGACACAA (bottom left panel), AATGGACA (bottom middle panel) or CGCGACAT (bottom right panel) were incubated in the absence (lanes 1, 9 and 19) or in the presence of increasing amounts (0.2 to 3.2 pmol) of recombinant GST-FOXP1 or GST-FOXP1-ES proteins. Positions mutated in the probe sequences are underlined. Shifted protein-dsDNA complexes are indicated by arrows and free dsDNA probe is indicated by an asterisk. Additional EMSA experiments employing other PBM-derived preferred binding sites for FOXP1 and FOXP1-ES are shown in FIG. 11B.

FIG. 3 shows that knockdown of FOXP1 and FOXP1-ES affects the expression of distinct sets of genes in hESCs.

FIG. 3(A) is a RT-PCR analysis of FOXP1 and FOXP1-ES splice isoforms in H9 hESCs transfected with a control, non targeting siRNA pool (lane 1), an siRNA pool targeting exon 18b (lane 2) and an siRNA pool targeting exon 18 (lane 3). ACTB mRNA levels are shown as a loading/recovery control.

FIG. 3(B) (Top) is a Venn diagram showing the numbers of genes with estimated 2-fold to 10.8-fold transcript level changes between the FOXP1 (black circle) or FOXP1-ES (light grey circle) knockdowns and the control knockdown samples shown in (A). FIG. 1(B) (Bottom) is a bar graph showing the proportions of genes showing up- (black fill) or down-regulation (white fill) in the gene sets affected by siRNA knockdown of exon 18 or exon 18b-containing transcripts. Genes with transcript changes affected in both knockdowns are also indicated (bar with grey outline).

FIG. 3(C) shows a Gene Ontology (GO) category enrichment analysis performed on sets of genes displaying increased or decreased transcript levels following siRNA knockdown of exon 18 and exon 18b-containing splice isoforms. Genes expressed in H9 hESCs were used as the comparison set in the GO analysis. The top four enriched annotations are shown for each gene set with their corresponding p-values, corrected using the Benjamini false discovery rate.

FIG. 3(D) shows qRT-PCR assays validating RNA-Seq predictions (FIG. 12C) of >˜2 fold changes in transcript levels from the pluripotency-associated genes (OCT4, TDGF1, NR5A2, NANOG, GDF3, FGF4) and differentiation-associated genes (GAS1, CITED2, WNT1, HESX1, BIK and SFRP4) following siRNA knockdown of FOXP1-ES and FOXP1 in H9 hESCs. Changes in expression are relative levels detected with a control siRNA pool. Expression ratios represent averages from three independent analyses and SDs are indicated.

FIG. 4 shows chromatin immunoprecipitation-high throughput sequencing analysis of FOXP1/FOXP1-ES target genes in hESCs.

FIG. 4(A) shows chromatin immunoprecipitation-high throughput sequencing (ChIP-Seq) analysis of FOXP1/FOXP1-ES binding sites in H9 hESCs was performed using a pan-FOXP1 isoform-specific antibody. The scatterplots compare relative enrichment scores for PBM-derived FOXP1 and FOXP1-ES 8-mer binding sequences under ChIP-Seq peaks, and PBM measured binding strengths. Z-scores were calculated by counting motif occurrences in peak sequences relative occurrences after randomizing the same peak sequences 100,000 times. PBM 8-mer sequences that bind preferentially to FOXP1, FOXP1-ES, or both proteins are marked as in FIG. 2B.

FIG. 4(B) shows representative tracks showing locations of FOXP1/FOXP1-ES ChIP-Seq peaks proximal (+/−20 kb of the transcription start site) to genes that display a ˜2 fold or greater change in mRNA expression upon knockdown of FOXP1 isoforms.

FIG. 4(C) is a bar graph representing the percentage of genes up- or down-regulated in response to FOXP1 or FOXP1-ES siRNA knockdown in H9 hESCs, which are experimentally-supported (based on combined ChIP and knockdown-expression analysis (Kunarso et al., 2010)) targets of OCT4. OCT4 target genes significantly overlap those genes showing decreased but not increased expression following knockdown FOXP1-ES (p=0.0016; Chi-square test), and do not significantly overlap genes with changes in expression following knockdown of FOXP1.

FIG. 5 shows that expression of Foxp1-ES but not Foxp1 promotes pluripotency maintenance of mESCs.

FIG. 5(A) shows CGR8 mESC lines expressing 3×Flag-Foxp1 or 3×Flag-Foxp1-ES under Doxycycline (Dox) inducible control, and the parental line used to generate these two cell lines (CGR8-rTA) were aggregated to form embryoid bodies (EBs) and then cultured under conditions promoting neural differentiation. The cultured EBs were treated with or without Dox and then immunostained for β-III tubulin (neural marker) or Oct4 (pluripotency marker). Nuclei were stained with Hoechst.

FIG. 5(B) is a quantification of CGR8 mESC proliferation in response to Dox-induced expression of 3×Flag-Foxp1 or 3×Flag-Foxp1-ES in the presence of excess LIF (LIF 1:1) which promotes mESC self-renewal, or in the presence of concentrations of LIF that are suboptimal for mESC self-renewal (LIF 1:10). Left panels: the plots show cell growth rates calculated as the cumulative difference in cell cycle numbers relative to the control condition (LIF1:1) without Dox-induced expression of the 3×Flag-Foxp1(-ES) transgenes. Right panel: quantification of the proportions of cells expressing Oct4 under the different growth conditions indicated after 4 cell passages. Quantifications represent two independent experiments and standard deviations are indicated.

FIG. 5(C) is a qRT-PCR analysis of transcript expression from genes involved in pluripotency maintenance in Dox-treated CGR8 mESCs expressing 3×Flag-Foxp1-ES and grown in absence of LIF (ΔLIF) for 24 passages. Average expression levels of Oct4, Nanog, Nr5a2, Sox2, Klf4 and LifR in CGR8 3×Flag-Foxp1-ES ΔLIF cells are shown relative to the average expression levels of the same genes in the parental CGR8 mESCs, cultured in parallel in the presence of 1:1 LIF. The expression ratios represent average measurements from three independent analyses, and positive SDs are indicated.

FIG. 5(D) is a teratoma assay assessing the pluripotency potential of mouse CGR8 3×Flag-Foxp1-ES ΔLIF cells (see panel C). Haematoxylin and eosin staining of teratoma sections detected all three embryonic germ layer-derived tissues. Endodermal derivatives: ciliated respiratory (a) and intestine-like (b) epithelium; mesodermal derivatives: muscle (c) and cartilage (d); ectodermal derivatives: neuronal (e) and skin epithelial cell (f). Bar=

FIG. 6 shows that Foxp1-ES is required for efficient reprogramming of MEFs into iPS cells.

FIG. 6(A) is a semi-quantitative RT-PCR analysis of the endogenous expression levels of Foxp1, Foxp1-ES, Oct4 and Sox2 during the course of reprogramming of secondary MEF-6C cells into secondary iPSC colonies (2°-6C iPSCs). Induction of Oct4, Klf4, cMyc and Sox2 transcription factors by addition of Dox at day 0 (2°-6C MEFs) was followed by monitoring transcript levels 2, 5, 11, 16, 21 and 30 days (2°-6C iPSCs) post Dox induction. Gapdh mRNA levels are shown as a loading/recovery control.

FIG. 6(B) is a bar graph showing the relative levels of expression of endogenous transcripts encoding Foxp1 and Foxp1-ES during the time course of reprogramming of 2°-6C MEFs. The levels of expression of Foxp1 and Foxp1-ES were normalized to Gapdh expression levels at each time point, and represented as log 2 ratios relative to the levels of Foxp1 and Foxp1-ES detected in 2°-6C MEFs and 2°-6C iPSCs, respectively. Positive SDs are indicated.

FIG. 6(C) is a bar graph showing the relative expression of Foxp1 and Foxp1-ES isoforms following transfection of siRNA pools. Cells were either mock transfected or transfected with siRNA pools specific for Foxp1 exon 16, Foxp1-ES exon 16b, or siRNA pools specific for Oct4. Expression levels were determined by semi-quantitative RT-PCR assays, normalized to Gapdh levels and relative to the expression levels of the same transcripts in the mock transfected control. Positive SDs are indicated.

FIG. 6(D) is a bar graph showing the relative proportions of flow cytometry-sorted, reprogramming 2°-6C MEFs that are double-positive for GFP and the ESC/iPSC marker SSEA-1. 2°-6C MEFs were Dox treated to induce OKMS factors and transfected with siRNA pools indicated in panel C at day 0, and then were analyzed by flow cytometry and immunostaining five days later. Results from analyzing the effects of transfecting the same siRNA pools at day 13 of reprogramming are shown in FIGS. 14C and D.

FIG. 6(E) shows representative images of SSEA-1 and DAPI-stained cells at day 5 following Dox induction of OKMS factors and post-transfection of siRNA pools as described in 5(C, D).

FIG. 7 shows the identification of regulatory factors for human FOXP1 exon 18b/mouse Foxp1 exon 16b alternative splicing.

FIG. 7A shows the analysis of 300 nucleotides upstream and downstream of FOXP1 exon 18b using the splicing code (Barash et al. 2010), and MBNL-binding motifs, U-rich motifs and other regulatory elements were predicted to control exon 18b splicing.

FIG. 7B shows that MBNL1/2 and TIA1/TIAL1 regulate alternative splicing of FOXP1 in H9 human ESCs and CGR8 mouse ESCs. RT-PCR assays monitoring alternative splicing patterns of FOXP1 in H9 and CGR8 cells following siRNA knockdown. Percent human 18b/mouse 16b exon inclusion levels (ES % inc) are shown below gel images. Expression levels of ACTIN and Gapdh transcripts are shown as loading controls.

FIG. 7C shows that MBNL1/2 regulate alternative splicing of FOXP1 in human 293T and mouse Neuro2A cells.

FIG. 7D shows the construction of wild type and mutant FOXP1 splicing reporters to test the regulation of FOXP1 alternative splicing in H9 human ESCs.

FIG. 8 shows a model for the role of alternative splicing in controlling transcriptional networks required for the regulation of ESC pluripotency and differentiation. In pluripotent ESCs or iPS cells, the inclusion of human FOXP1 exon 18b (or mouse Foxp1 exon 16b) results in the expression of FOXP1-ES, which preferentially binds to a distinct set of DNA motifs (FIG. 2). This event promotes the expression of transcription factors including OCT4 and NANOG required for the maintenance of pluripotency, and also represses genes required for ESC differentiation. The onset of differentiation triggers an alternative splicing shift resulting in complete skipping of exon 18b (exon 16b in mouse), the exclusive inclusion of exon 18 (exon 16 in mouse), and the expression of the “canonical” form of FOXP1 protein which preferentially binds to motifs with the consensus GTAAACA. Without being bound by theory, the loss of FOXP1-ES expression and possibly also the increased expression of FOXP1 results in reduced expression of pluripotency genes and increased expression of genes required for differentiation.

FIG. 9 is a quantitative gene expression analysis of lineage-specific markers in self renewing and differentiating H9 hESCs. qRT-PCR assays were used to analyzed changes in expression of nine lineage-specific markers in undifferentiated H9 hESCs (grown with Matrigel) and in H9 hESCs induced to differentiate into primitive endoderm (day 2), primitive mesoderm (day 2) and neural lineages (Neural Progenitor Cells [NPCs], days 2 and 10). Expression changes are represented as ratios over levels measured in undifferentiated H9 hESCs. Measurements are representative of three separate analyses; error bars indicate positive standard deviations.

(A) Endoderm markers: GATA6 and SOX17

(B) Mesoderm markers: BRACHYURY (T) and GOUSCOID (GSC)

(C) Neural markers: SOX1 and SOX3

(D) Pluripotency markers: OCT4, NANOG and SOX2

FIG. 10 is a comparison of human and mouse FOXP1/Foxp1 splice variants.

FIG. 10A (Top) is a schematic representation of human FOXP1 protein domains (encoded by the transcript NM_{—}032682) shown relative to coding exon boundaries, and overview of human FOXP1 splice variants (Updated from Brown et al., 2008). [ [symbol] ] exons overlap the 5″-UTR; [ [symbol] ] exons contain the translation initiation site; [▪] exons contain the translation termination site; exons annotated with an asterisk are truncated such that the resulting mRNA encodes only the first 21 amino acids of the forkhead domain; the [ [symbol] ] exon, when spliced in mRNA with exon 18, is predicted to introduce a premature termination codon (PTC) targeting the corresponding mRNA for degradation by the nonsense-mediated mRNA decay pathway.

FIG. 10A (Bottom) is a schematic representation of mouse Foxp1 protein domains (encoded by the transcript NM_{—}053202) shown relative to coding exon boundaries, and overview of mouse Foxp1 splice variants (Updated from Wang et al., 2003).

FIG. 10B is a table comparing length and sequence conservation between human FOXP1 exons 17 to 19 and the orthologous mouse Foxp1 exons 15 to 17.

FIG. 10C is a RT-PCR analysis of Foxp1 alternative splicing in mES R1 self-renewing cells (lane 1), in R1 cells aggregated to form embryoid bodies (EB) 5 days (lane 2) and 8 days (lane 3) post induction of differentiation, in R1 cells differentiated into beating cardiomyocytes (day 13, lane 4), and in R1-derived neurospheres (lane 5). The positions of primers used for RT-PCR assays are indicated by black arrows. Gapdh mRNA levels are shown for comparison.

FIG. 11 shows protein binding microarrays which reveal distinct DNA binding specificities for FOXP1 and FOXP1-ES.

FIG. 11A shows the full data used to generate the scatterplot in FIG. 2.

FIG. 11B shows an Electrophoresis Mobility Shift Assay (EMSA) validating PBM-derived 8-mer motifs predicted to bind with high affinity to FOXP1-ES (CAACACAA, ATACAAAA, CTAAACAA) and to both FOXP1 isoforms (AACAACAA). Radiolabeled dsDNA probes containing two copies of each 8-mer (top panels), or two copies of mutant versions of these sequences CCACTCAA, ATGCAGGA, CTATCCAA and AACCCCAA (bottom panels) were incubated in the absence (lanes 1, 10, 19 and 28) or in the presence of increasing amounts (0.2 to 3.2 pmol) of recombinant GST-FOXP1 or GST-FOXP1-ES proteins. Positions mutated in the probe sequences are underlined. Shifted protein-dsDNA complexes are indicated by arrows and free dsDNA probe is indicated by an asterisk.

FIG. 12 shows an RNA-Seq analysis of transcript level changes in FOXP1 and FOXP1-ES knockdowns.

FIG. 12A is a Western blot analysis of FOXP1 and FOXP1-ES levels in H9 hESCs following transfection with siRNA pools directed against exon 18 (lane 4) or exon 18b (lane 5), or with a control non-targeting siRNA pool (lane 3). Detection of FOXP1 in HEK293T and HeLa cell lysates is shown for comparison. Polymerase II (large subunit) levels are shown as a control for sample leading and recovery.

FIG. 12B is a scatterplot comparing the fold expression changes for 19 genes measured using qRT-PCR (x axis) and from RNA-Seq read analysis (y axis). The black () and grey ( [symbol] ) dots represent fold changes in transcript levels detected in H9 hESC RNA samples following knockdown using exon 18- and exon 18b-siRNAs pools, respectively, relative to H9 hESCs transfected with a non-targeting control siRNA pool. Comparison of the RNA-Seq and qRT-PCR measurements yields a correlation of 0.941.

FIG. 12C shows RNA-Seq predicted mRNA expression changes for key pluripotency genes and genes involved in differentiation following siRNA knockdown of FOXP1 and FOXP1-ES isoforms. The bar graph shows log 2 ratios of reads per kb per million reads (RPKM) values for each gene in H9 hESCs transfected with siRNA pools specific for FOXP1 exon 18- (black bars, ▪) or exon 18b- (grey bars, [symbol] ), relative to H9 hESCs transfected with a non-targeting control siRNA pool.

FIG. 13 shows the differentiation potential of transgenic mESC lines expressing Flag-tagged Foxp1 and Foxp1-ES under Dox-inducible control or expressing shRNAs directed against Foxp1 and Foxp1-ES.

FIG. 13A is a Western blot analysis using anti-Flag antibody reveals that 3×Flag-tagged Foxp1/Foxp1-ES proteins are detected in the presence but not in the absence of Dox induction in transgenic 3×Flag-Foxp1 and 3×Flag-Foxp1-ES CGR8 lines. Addition of 0.25 μg/ml Dox to cell culture medium resulted in an approximate two-fold increase in the overall level of each Foxp1 isoform, as compared to level of Foxp1 protein in the absence of Dox. Detection was performed with antibody specific for endogenous Foxp1. Bands corresponding to endogenous and Flag-tagged Foxp1/Foxp1-ES proteins are indicated by white and black arrows, respectively.

FIG. 13B shows immunostaining for β-III tubulin and Oct4 in differentiated EBs generated from CGR8 3×Flag-Foxp1-ES ΔLIF cells, in the absence or presence of Dox. Nuclei were stained with Hoechst.

FIG. 13C shows RT-PCR analysis of Foxp1 isoform expression in CGR8 cells infected with lentiviruses expressing a control shRNA directed against the GFP cDNA sequence (lane 1), an shRNA targeting exon 16 (lane 2), and an shRNA targeting exon 16b (lane 3). Gapdh mRNA levels are shown as loading control.

FIG. 13D is a bar graph quantifying alkaline phosphatase (AP) positive colonies observed after seeding 100 cells corresponding to lines expressing a control shRNA (CGR8-shRNA_control), an shRNA targeting Foxp1 exon 16 (-shRNA_ex16) and an shRNA targeting Foxp1 exon 16b (-shRNA_{—}ex16b). The bars represent the average number of colonies observed in 6 independent experiments, and positive standard deviations are indicated.

FIG. 13E shows representative images of AP-positive mESC colonies formed after expression of a control shRNA directed against GFP (a, CGR8-shRNA_control), an shRNA targeting Foxp1 exon 16 (b, -shRNA_ex16) and an shRNA targeting Foxp1 exon 16b (c, -shRNA_ex16b).

FIG. 13F is a teratoma assay assessing the pluripotency potential of mouse CGR8 3×Flag-Foxp1-ES ΔLIF cells (see FIG. 5D). Periodic Acid-Schiff (PAS) staining (to detect glycoprotein expressing cells) and Safranin O staining (to detect cartilage) confirmed the presence of intestine-like epithelial cells (a, endodermal derivative) and cartilage (b, mesodermal derivative), respectively. The teratomas were also immunostained for GFAP (c) to confirm the presence of ectodermal derivatives. Nuclei were stained with DAPI. Scale=50 μM.

FIG. 14 shows that FOXP1-ES expression is required for efficient reprogramming of MEFs to iPSCs.

FIG. 14A is a RT-PCR analysis of the endogenous mRNA expression of Foxp1 and Foxp1-ES in MEFs and primary 6C iPSCs (lanes 1 and 2), as well as before and after reprogramming of secondary MEF cells into secondary iPS colonies (2°-6C iPSCs) (lanes 3 and 4) (see FIG. 2). RT-PCR analysis was performed using primers specific for exons 15 and 17 (black arrows). Percent exon inclusion levels are indicated.

FIG. 14B is a bar graph showing the relative expression of Foxp1 and Foxp1-ES isoforms in pre-iPSC colonies at day 16, following transfection at day 11 of control, exon 16- and exon 16b-targeting siRNA pools, as well as single siRNAs that comprise these pools. mRNA expression levels were determined by semi-quantitative RT-PCR assays, normalized to Gapdh levels, and relative to the expression levels of the same transcripts in the mock transfected control. Error bars indicate positive SDs.

FIG. 14C is a bar graph showing the relative proportions of flow cytometry-sorted, reprogramming 2°-6C MEFs that are double-positive for GFP and the ESC/iPSC marker SSEA-1. 2°-6C MEFs were Dox treated to induce expression of OKMS factors and GFP protein, transfected with siRNA pools at day 13 of reprogramming, then were analyzed by flow cytometry and immunostaining three days later. Bars indicate range from median values from two independent experiments.

FIG. 14D shows representative images (from several repeat experiments) of primary MEFs undergoing reprogramming for 9 days following ectopic overexpression of OKMS factors, with or without ectopic over-expression of Foxp1 or Foxp1-ES, as indicated above each plate. Plates were stained for alkaline phosphatase, or SSEA1 (not shown).

FIG. 15 shows low expression of MBLN1 and MBLN2 genes in ESCs relative to all other analyzed cell and tissue types, and splicing code features predicted to be important for ESC-specific alternative splicing. (a) expression of MBNL1 and MBNL2 genes across all studied cell types and tissues, based on qPCR data. ESCs (in grey) and differentiated cells and tissues (in black) (b) Splicing code features that are most significantly correlated with differences in Percent Spliced In (PSI; the percentage of transcripts with the exon spliced in) levels in ESCs compared to non-ESC lines and differentiated tissues are indicated. Features are ranked according to distance correlation p-values (y-axis), for exons with either lower (top) or higher (bottom) inclusion in ESCs.

FIG. 16 shows that RNAi-mediated knockdown of MBNL1 and MBNL2 promotes ESC-specific AS of FOXP1. a) Western blots confirming the efficient knockdown of MBNL1 and MBNL2 proteins in 293T cells transfected with siRNAs targeting these factors (lane 6), and with a non-targeting siRNA pool used as a control (lane 5). Lanes 1-4, serial dilutions of total protein cell lysate (1, 1:2, 1:4 and 1:8). b) Splicing code feature map highlighting genomic coordinates of MBNL features predicted to regulate inclusion of FOXP1 exon 18b. c) RT-PCR assays monitoring mRNA levels of FOXP1 isoforms in 293T cells transfected with control siRNAs or siRNAs targeting MBNL1, MBNL2 or both of these factors. Primers specific for splice junctions were designed to selectively amplify FOXP1 isoforms containing the ESC-specific exon 18b (“FOXP1-ES”) and exon 18 (“FOXP1”). Expression levels of ACTIN are shown as loading controls. d) mRNA levels of murine Foxp1-ES and Foxp1 isoforms were assayed in mouse CGR8 stem cells as in (c) following transfections of control siRNAs or siRNAs targeting Mbnl1, Mbnl2 or both of these factors in mouse N2A cells. Expression levels of Gapdh are shown as loading controls.

FIG. 17 shows that MBNL1 and MBNL2 regulate approximately half of analyzed exons differentially regulated between ESCs and differentiated cells and tissues (hereafter referred to as ESC-specific alternative splicing events). a) Venn diagram showing the overlap between ESC-specific alternative splicing events in genes expressed in HeLa cells (medium grey) and the exons that show ≧15 change in PSI in HeLa cells transfected with MBNL1+2 siRNAs and with control siRNA (pale grey). b) High correlation (r=0.90) between the differences in average PSI levels of exons between ESCs versus differentiated cells/tissues, and differences in PSI levels in HeLa cells following siRNA knockdown of MBNL1 and MBNL2 KD versus a control siRNA. c) Change in PSI upon MBNL1 and MBNL2 knockdown in HeLa cells for representative ESC-specific exons; PSI in H9 (a human ESC line) is shown for comparison. Exon-included and exon-skipped mRNA isoforms are indicated.

FIG. 18 shows that knockdown of Mbnl proteins enhances the expression of key pluripotency genes and formation of SSEA1+ colonies at an early stage of iPSC reprogramming a) Schematic of experimental setup and time points of qRT-PCR analysis (top); mRNA expression levels of Oct4, Nanog, Sall4 and Alpl were quantified by qRT-PCR following siRNA-mediated knockdown of Mbnl1 and Mbnl2 proteins and Dox induction of OKMS factors at day 3 and at day 5 post-induction, and plotted relative to the expression level of each gene in MOCK control at day 5 post-induction (bottom). Oct4 siRNA transfection was used as a negative control, and MEFs without Dox induction are shown as empty bars. Values represent means±SE (n=3). b) Quantification of SSEA1 stained area changes following siRNA-mediated knockdown of Mbnl1+Mbnl2 and Oct4 relative to siRNA control at Day 5 post-induction of OKMS factors with values representing means±SE (n=3) (top); representative images of SSEA1 and DAPI stained cells at Day 5 post-induction (bottom). c) Model for the role of MBNL/Mbnl proteins in ESC pluripotency and iPSC reprogramming.

### DETAILED DESCRIPTION OF THE DISCLOSURE

### (A) Nucleic Acids

The present inventors have demonstrated that an alternatively spliced variant of forkhead family transcription factor FOXP1 promotes the maintenance of embryonic stem cell pluripotency and the reprogramming of somatic cells to induced pluripotent stem cells.

Accordingly, an isolated nucleic acid molecule including exon 18b of the human FOXP1 gene (hereinafter referred to as hFOXP1-ES; SEQ ID NO: 3) is described. In another embodiment, an isolated nucleic acid molecule including exon 16b of the mouse Foxp1 gene (hereinafter referred to as mFOXP1-ES; SEQ ID NO: 7) is described. An isolated nucleic acid molecule comprising exon 18b (SEQ ID NO: 4) and an isolated nucleic acid molecule comprising exon 16b (SEQ ID NO: 8) are also described. The genomic coordinates for human exons 17, 18, 18b and 19 of hFOXP1 are given in Table 1 and the genomic coordinates for mouse exons 15, 16, 16b and 17 of mFoxp1 are given in Table 2.

The term “isolated” refers to a nucleic acid substantially free of cellular material or culture medium when produced by recombinant DNA techniques, or chemical precursors, or other chemicals when chemically synthesized.

The term “nucleic acid molecule” is intended to include unmodified DNA or RNA or modified DNA or RNA. For example, the nucleic acid molecules or polynucleotides of the disclosure can be composed of single- and double stranded DNA, DNA that is a mixture of single- and double-stranded regions, single- and double-stranded RNA, and RNA that is a mixture of single- and double-stranded regions, hybrid molecules comprising DNA and RNA that may be single-stranded or, more typically double-stranded or a mixture of single- and double-stranded regions. In addition, the nucleic acid molecules can be composed of triple-stranded regions comprising RNA or DNA or both RNA and DNA. The nucleic acid molecules of the disclosure may also contain one or more modified bases or DNA or RNA backbones modified for stability or for other reasons. “Modified” bases include, for example, tritiated bases and unusual bases such as inosine. A variety of modifications can be made to DNA and RNA; thus “nucleic acid molecule” embraces chemically, enzymatically, or metabolically modified forms. The term “polynucleotide” shall have a corresponding meaning.

The term “FOXP1” as used herein refers to the forkhead family transcription factor FOXP1 from any species or source, optionally mammalian, such as human or mouse. While the term “FOXP” is often used to here to human FOXP1, in the present disclosure, the term is also used to refer to FOXP1 from other species. “FOXP1-ES” refers to the FOXP1 gene from any species or source that includes an exon 18b or a homolog thereof, such as exon 16b of mouse.

“hFOXP1-ES” refers to the human FOXP1 gene that includes exon 18b and the protein produced by that gene, also described herein as “FOXP1-ES”. “mFoxp1-ES” refers to the mouse Foxp1 gene that includes exon 16b and the protein produced by that gene, also described herein as “Foxp1-ES”. While the term “FOXP1-ES” is often used to here to human FOXP1-ES, in the present disclosure, the term is also used to refer to FOXP1-ES from other species. FOXP1-ES refers to an ESC-specific isoform of FOXP1. FOXP1-ES also refers to an iPSC-specific isoform of FOXP1.

One aspect of the present disclosure is thus an isolated nucleic acid molecule comprising a nucleotide sequence selected from the group consisting of:
(a) a nucleic acid sequence as shown in SEQ ID NOS: 3, 4, 7 or 8 (hFOXP1-ES, exon 18b, mFoxp1-ES or exon 16b);
(b) a nucleic acid sequence that is complementary to a nucleic acid sequence of (a);
(c) a nucleic acid sequence that has substantial sequence identity to a nucleic acid sequence of (a) or (b);
(d) a nucleic acid sequence that hybridizes to a nucleic acid sequence of (a), (b) or (c) under stringent hybridization conditions; or
(e) a nucleic acid sequence differing from any of the nucleic acid sequences of (a) to (d) in codon sequences due to the degeneracy of the genetic code.

As previously stated, the disclosure includes isolated DNA molecules having such sequences of nucleotides and RNA molecules having such sequences. The disclosure thus includes isolated mRNA transcribed from DNA having such a sequence. The disclosure further encompasses nucleic acid molecules that differ from any of the nucleic acid molecules of the disclosure in codon sequences due to the degeneracy of the genetic code.

In another aspect, the present disclosure includes a fragment of the nucleotide sequence encoding hFOXP1-ES, said fragment comprising exon 18b (SEQ ID NO: 4). The present disclosure also includes a fragment of the nucleotide sequence encoding mFoxp1-ES, said fragment comprising exon 16b (SEQ ID NO: 8). Such fragments can find usefulness as probes or depending on the fragments may even have biological activity themselves. The complement of the probe can find utility in, for example, manufacture of the probe or inhibition of any activity of the fragment, as the case may be. In a particular use, the probe can be used to determine the presence of an RNA molecule in a sample which might, or might not, also include an RNA molecule encoding FOXP1-ES. Such a probe would generally be 20 nucleotides long or be at least 20 nucleotides long. The probe could also be 25, 30, 35, 40, 45, 50, 55, 60 or more nucleotides in length and the probe can include the full length of the complement to the sequence to which it is intended to bind.

The disclosure includes the method of determining the presence of a nucleic acid molecule encoding FOXP1-ES in a sample containing RNA isolated from a cell, using such a probe.

In the context of this specification, the term “conserved” describes similarity between sequences. The degree of conservation between two sequences can be determined by optimally aligning the sequences for comparison. Sequences may be aligned using the Omiga software program, Version 1.13 (Oxford Molecular Group, Inc., Campbell, Calif.). The Omiga software uses the Clustal W Alignment algorithms [Higgins et al., 1989; Higgins et al., 1991; Thompson et al. 1994] Default settings used are as follows: Open gap penalty 10.00; Extend gap penalty 0.05; Delay divergent sequence 40 and Scoring matrix—Gonnet Series. Percent identity or homology between two sequences is determined by comparing a position in the first sequence with a corresponding position in the second sequence. When the compared positions are occupied by the same nucleotide or amino acid, as the case may be, the two sequences are conserved at that position. The degree of conservation between two sequences is often expressed, as it is here, as a percentage representing the ratio of the number of matching positions in the two sequences to the total number of positions compared.

In one aspect, the present disclosure is a nucleic acid molecule which encodes a protein that is a conservatively substituted variant of the protein encoded by the nucleotide sequence of SEQ ID NOS: 3, 4, 7 or 8.

Further, it will be appreciated that the disclosure includes nucleic acid molecules comprising nucleic acid sequences having substantial sequence identity with the nucleic acid sequence as shown in SEQ ID NOS: 3, 4, 7 or 8 or fragments thereof. The term “sequences having substantial sequence identity” means those nucleic acid sequences that have slight or inconsequential sequence variations from these sequences, i.e., the sequences function in substantially the same manner to produce functionally equivalent proteins. The variations may be attributable to local mutations or structural modifications.

Nucleic acid sequences having substantial identity include nucleic acid sequences having at least about 50 percent identity with a protein encoded by SEQ ID NOS: 3, 4, 7 or 8, respectively, or the full-length anti-sense sequence thereto. The level of sequence identity, according to various aspects of the disclosure is at least about: 60, 70, 75, 80, 83, 85, 88, 90, 93, 95 or 98 percent. Methods for aligning the sequences to be compared and determining the level of homology between the sequences are described in detail above.

In one aspect, the nucleic acid molecule having substantial sequence identity described above has a sequence identity of at least about: 50, 60, 70, 75, 80, 83, 85, 88, 90, 93, 95 or 98 percent within the exon 18b (nucleic acid 1531 to 1720 of SEQ ID NO: 3 numbering from the first ATG of the human FOXP1-ES gene sequence) or exon 16b (nucleic acid 1615 to 1784 of SEQ ID NO:7 numbering from the first ATG of the mouse Foxp1-ES gene sequence).

The disclosure is not to be restricted by this sequence identity, for instance, nucleic acid sequences having at least a 50% sequence identity with the sequence shown in SEQ ID NOS: 3, 4, 7 or 8 are also encompassed within the scope of the present disclosure where there is at least 50%, at least 60%, at least 70%, at least 80%, at least 90%, at least 95% or at least 98% homology with the sequence from nucleic 1531 to 1720 of SEQ ID NO: 3 numbering from the first ATG of the human FOXP1-ES gene sequence nucleic acid 1615 to 1784 of SEQ ID NO:7 numbering from the first ATG of the mouse Foxp1-ES gene sequence.

Sequence identity can be calculated according to methods known in the art. Sequence identity is most preferably assessed by the algorithm of BLAST version 2.1 advanced search. BLAST is a series of programs that are available, for example, online from the National Institutes of Health. The advanced blast search is set to default parameters. (ie Matrix BLOSUM62; Gap existence cost 11; Per residue gap cost 1; Lambda ratio 0.85 default). References to BLAST searches are: Altschul, S. F., Gish, W., Miller, W., Myers, E. W. & Lipman, D. J. (1990) “Basic local alignment search tool.” J. Mol. Biol. 215:403410; Gish, W. & States, D. J. (1993) “Identification of protein coding regions by database similarity search.” Nature Genet. 3:266272; Madden, T. L., Tatusov, R. L. & Zhang, J. (1996) “Applications of network BLAST server” Meth. Enzymol. 266:131_{—}141; Altschul, S. F., Madden, T. L., Schäffer, A. A., Zhang, J., Zhang, Z., Miller, W. & Lipman, D. J. (1997) “Gapped BLAST and PSI_BLAST: a new generation of protein database search programs.” Nucleic Acids Res. 25:33893402; Zhang, J. & Madden, T. L. (1997) “PowerBLAST: A new network BLAST application for interactive or automated sequence analysis and annotation.” Genome Res. 7:649656.

The term “sequence that hybridizes” means a nucleic acid sequence that can hybridize to a sequence of (a), (b) or (c) under stringent hybridization conditions. Appropriate stringency conditions which promote nucleic acid hybridization are known to those skilled in the art, or may be found in Current Protocols in Molecular Biology, John Wiley & Sons, N.Y. (1989), 6.3.1-6.3.6. The term “stringent hybridization conditions” as used herein means that conditions are selected which promote selective hybridization between two complementary nucleic acid molecules in solution. Hybridization may occur to all or a portion of a nucleic acid sequence molecule. The hybridizing portion is at least 50% the length with respect to one of the polynucleotide sequences encoding a polypeptide. In this regard, the stability of a nucleic acid duplex, or hybrids, is determined by the Tm, which in sodium containing buffers is a function of the sodium ion concentration, G/C content of labeled nucleic acid, length of nucleic acid probe (I), and temperature (Tm=81.5° C.−16.6 (Log 10[Na+])+0.41(% (G+C)−600/I). Accordingly, the parameters in the wash conditions that determine hybrid stability are sodium ion concentration and temperature. In order to identify molecules that are similar, but not identical, to a known nucleic acid molecule a 1% mismatch may be assumed to result in about a 1° C. decrease in Tm, for example if nucleic acid molecules are sought that have a greater than 95% identity, the final wash will be reduced by 5° C. Based on these considerations stringent hybridization conditions shall be defined as: hybridization at 5× sodium chloride/sodium citrate (SSC)/5×Denhardt's solution/1.0% SDS at Tm (based on the above equation)-5° C., followed by a wash of 0.2×SSC/0.1% SDS at 60° C.

Isolated nucleic acid molecules having sequences which differ from the nucleic acid sequence shown in SEQ ID NOS: 3, 4, 7 or 8 due to degeneracy in the genetic code are also within the scope of the disclosure. Such nucleic acids encode functionally equivalent proteins or peptides but differ in sequence from the above mentioned sequences due to degeneracy in the genetic code.

An isolated nucleic acid molecule of the disclosure which comprises DNA can be isolated by preparing a labelled nucleic acid probe based on all or part of the nucleic acid sequences as shown in SEQ ID NOS: 3, 4, 7 or 8 and using this labelled nucleic acid probe to screen an appropriate DNA library (e.g. a cDNA or genomic DNA library).

An isolated nucleic acid molecule of the disclosure which is DNA can also be isolated by selectively amplifying a nucleic acid encoding a novel protein of the disclosure using the polymerase chain reaction (PCR) methods and cDNA or genomic DNA. It is possible to design synthetic oligonucleotide primers from the nucleic acid sequences shown in SEQ ID NOS: 3, 4, 7 or 8 for use in PCR. A nucleic acid can be amplified from cDNA or genomic DNA using these oligonucleotide primers and standard PCR amplification techniques. The nucleic acid so amplified can be cloned into an appropriate vector and characterized by DNA sequence analysis. It will be appreciated that cDNA may be prepared from mRNA, by isolating total cellular mRNA by a variety of techniques, for example, by using the guanidinium-thiocyanate extraction procedure of Chirgwin et al., Biochemistry, 18, 5294 5299 (1979). cDNA is then synthesized from the mRNA using reverse transcriptase (for example, Moloney MLV reverse transcriptase available from Gibco/BRL, Bethesda, Md., or AMV reverse transcriptase available from Seikagaku America, Inc., St. Petersburg, Fla.).

An isolated nucleic acid molecule of the disclosure which is RNA can be isolated by cloning a cDNA encoding a novel protein of the disclosure into an appropriate vector which allows for transcription of the cDNA to produce an RNA molecule which encodes a protein of the disclosure. For example, a cDNA can be cloned downstream of a bacteriophage promoter, (e.g., a T7 promoter) in a vector, cDNA can be transcribed in vitro with T7 polymerase, and the resultant RNA can be isolated by standard techniques.

A nucleic acid molecule of the disclosure may also be chemically synthesized using standard techniques. Various methods of chemically synthesizing polydeoxynucleotides are known, including solid-phase synthesis which, like peptide synthesis, has been fully automated in commercially available DNA synthesizers (See e.g., Itakura et al. U.S. Pat. No. 4,598,049; Caruthers et al. U.S. Pat. No. 4,458,066; and Itakura U.S. Pat. Nos. 4,401,796 and 4,373,071).

The sequence of a nucleic acid molecule of the disclosure may be inverted relative to its normal presentation for transcription to produce an antisense nucleic acid molecule. The term “antisense” nucleic acid molecule is a nucleotide sequence that is complementary to its target. Preferably, an antisense sequence is constructed by inverting a region preceding or targeting the initiation codon or an unconserved region. In another embodiment the antisense sequence targets all or part of the mRNA or cDNA of FOXP1-ES or FOXP1. In particular, the nucleic acid sequences contained in the nucleic acid molecules of the disclosure or a fragment thereof may be inverted relative to its normal presentation for transcription to produce antisense nucleic acid molecules. In one embodiment the antisense molecules can be used to inhibit FOXP1-ES or FOXP1 expression.

The antisense nucleic acid molecules of the disclosure or a fragment thereof, may be chemically synthesized using naturally occurring nucleotides or variously modified nucleotides designed to increase the biological stability of the molecules or to increase the physical stability of the duplex formed with mRNA or the native gene e.g. phosphorothioate derivatives and acridine substituted nucleotides. The antisense sequences may be produced biologically using an expression vector introduced into cells in the form of a recombinant plasmid, phagemid or attenuated virus in which antisense sequences are produced under the control of a high efficiency regulatory region, the activity of which may be determined by the cell type into which the vector is introduced.

In another embodiment, the disclosure provides interfering RNA molecules such as siRNAs, shRNAs and miRNAs that target and silence FOXP1 or FOXP1-ES. Accordingly, in one aspect, the disclosure provides siRNAs for knockdown of human FOXP1 comprising SEQ ID NOs: 25-28, human FOXP1-ES comprising SEQ ID NOs: 29-32, mouse FOXP1 comprising SEQ ID NOs: 17-20 and mouse FOXP1-ES comprising SEQ ID NOs: 21-24. In another aspect, the disclosure provides shRNAs for knockdown of mouse Foxp1 isoforms (SEQ ID NOs: 33-35).

The disclosure also provides an isolated nucleic acid molecule comprising an antisense oligonucleotide to the nucleic acid sequence as shown in any one of SEQ ID NOS: 36-43. Optionally, the antisense oligonucleotide is between 1 and 300 nucleotides in length, optionally 2-50, 2-30 or 5-20 nucleotides in length. In one embodiment, the antisense oligonucleotide is optionally 2 to 50, 5 to 40 or 10 to 25 nucleotides in length.

The disclosure also provides a kit for modulating the expression of FOXP1-ES wherein the kit comprises at least two antisense oligonucleotides to the nucleic acid sequence as shown in any one of SEQ ID NOS: 36-43.

### (B) Proteins/Polypeptides

The present application further contemplates an isolated FOXP1-ES polypeptide, human FOXP1-ES polypeptide (SEQ ID NO: 11) or mouse Foxp1-ES polypeptide (SEQ ID NO: 15) or homologs thereof. In an embodiment of the disclosure, an isolated polypeptide is provided which has the amino acid sequence as shown in SEQ ID NO:11 or a fragment, having retained the amino acids encoded by exon 18b, thereof. In another embodiment of the disclosure, an isolated polypeptide is provided which has the amino acid sequence as shown in SEQ ID NO:15 or a fragment, having retained the amino acids encoded by exon 16b, thereof. The present disclosure also encompasses peptides comprising the amino acid sequence shown in SEQ ID NOs: 12 (exon 18b) and 16 (exon 16b).

Within the context of the present application, a polypeptide of the disclosure may in one embodiment include various structural forms of the primary protein. For example, a polypeptide of the disclosure may be in the form of acidic or basic salts or in neutral form. In addition, individual amino acid residues may be modified by oxidation or reduction.

In addition to the full-length amino acid sequences corresponding to human FOXP1-ES and mouse Foxp1-ES (SEQ ID NOs: 11 and 15, respectively), the polypeptides of the present disclosure may also include truncations of the polypeptides, and analogs, and homologs of the proteins and truncations thereof as described herein. Truncated polypeptides may comprise peptides of at least 10 and preferably at least fourteen amino acid residues.

In an embodiment, the disclosure provides a peptide fragment comprising amino acids 511 to 565 of SEQ ID NO: 11 or an analog or homolog thereof. In one embodiment, the disclosure provides a peptide fragment comprising the amino acid sequence shown in SEQ ID NO: 12 or an analog or homolog thereof.

In an embodiment, the disclosure provides a peptide fragment comprising amino acids 538 to 594 of SEQ ID NO: 15 or an analog or homolog thereof. In one embodiment, the disclosure provides a peptide fragment comprising the amino acid sequence shown in SEQ ID NO: 16 or an analog or homolog thereof.

Analogs of the proteins having the amino acid sequences shown in SEQ ID NO: 11 or 15 as described herein, may include, but are not limited to an amino acid sequence containing one or more amino acid substitutions, insertions, and/or deletions. Amino acid substitutions may be of a conserved or non-conserved nature. Conserved amino acid substitutions involve replacing one or more amino acids of the proteins of the disclosure with amino acids of similar charge, size, and/or hydrophobicity characteristics. When only conserved substitutions are made the resulting analog should be functionally equivalent. Non-conserved substitutions involve replacing one or more amino acids of the amino acid sequence with one or more amino acids which possess dissimilar charge, size, and/or hydrophobicity characteristics.

Conservative substitutions are described in the patent literature, as for example, in U.S. Pat. No. 5,264,558. It is thus expected, for example, that interchange among non-polar aliphatic neutral amino acids, glycine, alanine, proline, valine and isoleucine, would be possible. Likewise, substitutions among the polar aliphatic neutral amino acids, serine, threonine, methionine, asparagine and glutamine could possibly be made. Substitutions among the charged acidic amino acids, aspartic acid and glutamic acid, could probably be made, as could substitutions among the charged basic amino acids, lysine and arginine. Substitutions among the aromatic amino acids, including phenylalanine, histidine, tryptophan and tyrosine would also likely be possible. Other substitutions might well be possible.

One or more amino acid insertions may be introduced into the amino acid sequences shown in SEQ ID NO: 11 or 15. Amino acid insertions may consist of single amino acid residues or sequential amino acids ranging from 2 to 15 amino acids in length. This procedure may be used in vivo to inhibit the activity of FOXP1-ES.

Deletions may consist of the removal of one or more amino acids, or discrete portions from the amino acid sequence shown in SEQ ID NO: 11, excluding the region corresponding to exon 18b (511 to 565 of SEQ ID NO: 11) or from the amino acid sequence shown in SEQ ID NO:15, excluding the region corresponding to exon 16b (amino acids 538 to 594 of SEQ ID NO: 15). The deleted amino acids may or may not be contiguous. The lower limit length of the resulting analog with a deletion mutation is about 10 amino acids, preferably 100 amino acids.

Exemplary methods of making the alterations set forth above are disclosed by Sambrook et al (Molecular Cloning: A Laboratory Manual, 2nd Ed., Cold Spring Harbor Laboratory Press, 1989).

The proteins of the disclosure also include homologs of the amino acid sequence shown in SEQ ID NO: 11 or 15 and/or truncations thereof as described herein. Such homologs are proteins whose amino acid sequences are encoded by nucleic acid sequences that hybridize under stringent hybridization conditions (see discussion of stringent hybridization conditions herein) with a probe used to obtain a protein of the disclosure. Homologs of a protein of the disclosure will have the same regions which are characteristic of the protein.

A homologous protein includes a protein with an amino acid sequence having at least 60%, at least 70%, at least 80%, at least 90%, at least 95% or at least 98% identity with the amino acid sequence as shown in SEQ ID NO: 11 or 15.

The present application also includes a protein described above conjugated with a selected protein, or a selectable marker protein (see below) to produce fusion proteins.

The proteins described above (including truncations, analogs, etc.) may be prepared using recombinant DNA methods. These proteins may be purified and/or isolated to various degrees using techniques known in the art. Accordingly, nucleic acid molecules of the present disclosure having a sequence which encodes a protein of the disclosure may be incorporated according to procedures known in the art into an appropriate expression vector which ensures good expression of the protein. Possible expression vectors include but are not limited to cosmids, plasmids, or modified viruses (e.g., replication defective retroviruses, adenoviruses and adeno-associated viruses), so long as the vector is compatible with the host cell used. The expression “vectors suitable for transformation of a host cell”, means that the expression vectors contain a nucleic acid molecule of the disclosure and regulatory sequences, selected on the basis of the host cells to be used for expression, which are operatively linked to the nucleic acid molecule. “Operatively linked” is intended to mean that the nucleic acid is linked to regulatory sequences in a manner which allows expression of the nucleic acid.

The disclosure therefore contemplates a recombinant expression vector of the disclosure containing a nucleic acid molecule of the disclosure, or a fragment thereof, and the necessary regulatory sequences for the transcription and translation of the inserted protein-sequence. The disclosure further provides a recombinant expression vector comprising a DNA nucleic acid molecule of the disclosure cloned into the expression vector in an antisense orientation. That is, the DNA molecule is operatively linked to a regulatory sequence in a manner which allows for expression, by transcription of the DNA molecule, of an RNA molecule which is antisense to a nucleotide sequence of the disclosure optionally comprising the nucleotides as shown in SEQ ID NO: 3, 4, 7 or 8 or fragments thereof. Regulatory sequences operatively linked to the antisense nucleic acid can be chosen which direct the continuous expression of the antisense RNA molecule.

The recombinant expression vectors of the disclosure may also contain a selectable marker gene that facilitates the selection of host cells transformed or transfected with a recombinant molecule of the disclosure. Examples of selectable marker genes are genes encoding a protein which confers resistance to certain drugs, such as G418 and hygromycin.

Recombinant expression vectors can be introduced into host cells to produce a transformed host cell. The term “transformed host cell” is intended to include prokaryotic and eukaryotic cells which have been transformed or transfected with a recombinant expression vector of the disclosure. The terms “transformed with”, “transfected with”, “transformation” and “transfection” are intended to encompass introduction of nucleic acid (e.g. a vector) into a cell by one of many possible techniques known in the art.

Suitable host cells include a wide variety of prokaryotic and eukaryotic host cells. For example, the proteins of the disclosure may be expressed in bacterial cells such as E. coli, insect cells (using baculovirus), yeast cells or mammalian cells, COS1 cells. Other suitable host cells can be found in Goeddel, Gene Expression Technology: Methods in Enzymology 185, Academic Press, San Diego, Calif. (1991).

The disclosure includes a microbial cell that contains and is capable of expressing a heterologous a nucleic acid molecule having a nucleotide sequence as encompassed by the disclosure. The heterologous nucleic acid molecule can be DNA.

Isolated DNA of the disclosure can be contained in a recombinant cloning vector.

The disclosure includes a stably transfected cell line which expresses any one or more proteins as defined by the disclosure.

The disclosure includes a culture of cells transformed with a recombinant DNA molecule having a nucleotide sequence as encompassed by the disclosure.

The application also contemplates a process for producing any protein as defined by the disclosure. The process includes such steps as:

preparing a DNA fragment including a nucleotide sequence which encodes said protein;

incorporating the DNA fragment into an expression vector to obtain a recombinant DNA molecule which includes the DNA fragment and is capable of undergoing replication;

transforming a host cell with said recombinant DNA molecule to produce a transformant which can express said protein;

culturing the transformant to produce said protein; and

recovering said protein from resulting cultured mixture.

More particularly, the application provides a method of preparing a purified protein of the disclosure comprising introducing into a host cell a recombinant nucleic acid encoding the protein, allowing the protein to be expressed in the host cell and isolating and purifying the protein.

Alternatively, the protein or parts thereof can be prepared by chemical synthesis using techniques well known in the chemistry of proteins such as solid phase synthesis or synthesis in homogeneous solution.

### (C) Binding Proteins

The application provides binding proteins that bind to the FOXP1-ES protein, but do not bind to the FOXP1 protein.

The term “binding protein” as used herein refers to proteins that specifically bind to another substance. In an embodiment, the binding proteins bind to the human FOXP1-ES protein, but not to the human FOXP1 protein. Accordingly, in one embodiment, the binding protein binds to the protein having the amino acid sequence shown in SEQ ID NO:11. In another embodiment, the binding protein binds to a protein encoded by the nucleic acid sequence as shown in SEQ ID NO: 3. In another embodiment, the binding proteins bind to the mouse Foxp1-ES protein, but not to the mouse Foxp1 protein. Accordingly, in one embodiment, the binding protein binds to the protein having the amino acid sequence shown in SEQ ID NO: 15. In another embodiment, the binding protein binds to a protein encoded by the nucleic acid sequence as shown in SEQ ID NO:7. In another embodiment, the binding proteins are antibodies or antibody fragments thereof. In a further embodiment, the binding proteins are monoclonal antibodies or fragments thereof. The term “antibody” as used herein is intended to include monoclonal antibodies, polyclonal antibodies, and chimeric antibodies. The antibody may be from recombinant sources and/or produced in transgenic animals. The term “antibody fragment” as used herein is intended to include Fab, Fab′, F(ab′)₂, scFv, dsFv, ds-scFv, dimers, minibodies, diabodies, and multimers thereof and bispecific antibody fragments. Antibodies can be fragmented using conventional techniques.

In one embodiment of the disclosure, the binding protein binds to an epitope on the FOXP1-ES protein comprising the portion that is encoded by exon 18b which is normally not found in the wild type FOXP1 protein. For example, in one embodiment of the disclosure the epitope comprises the amino acid sequence as shown in SEQ ID NO:12, or a fragment thereof. Accordingly, in another embodiment, the binding protein binds to an epitope on the mouse Foxp1-ES protein comprising the portion that is encoded by exon 16b which is normally not found in the wild type mouse Foxp1 protein. For example, in one embodiment of the disclosure the epitope comprises the amino acid sequence as shown in SEQ ID NO:16, or a fragment thereof.

The term “epitope” as used herein refers to the part of the protein which contacts the antigen binding site of the binding protein of the disclosure.

The fragments of FOXP1-ES described above are useful as antigens in preparing antibodies that bind to the protein portion encoded by FOXP1-ES but do not bind to the FOXP1. In a particular embodiment, the antigen comprises the unique sequence in human FOXP1-ES, which is encoded by exon 18b as shown in SEQ ID NO. 4. In another embodiment, the antigen comprises the unique sequence in mouse Foxp1-ES which is encoded by exon 16b as shown in SEQ ID NO. 8.

In an embodiment, the disclosure provides a method of making an antibody that binds to FOXP1-ES comprising the following steps:
a) immunizing a host with an immunogen comprising an isolated FOXP1-ES fragment having the amino acid sequence as shown in SEQ ID NO:12;
b) isolating an antibody from said host that binds FOXP1-ES.

In an embodiment, the disclosure provides a method of making an antibody that binds to mouse FOXP1-ES comprising the following steps:
a) immunizing a host with an immunogen comprising an isolated mouse FOXP1-ES fragment having the amino acid sequence as shown in SEQ ID NO:16;
b) isolating an antibody from said host that binds mouse FOXP1-ES.

Conventional methods are useful to prepare the antibodies. For example, by using a peptide of a protein of the disclosure, polyclonal antisera or monoclonal antibodies can be made using standard methods.

Specific antibodies, or antibody fragments reactive against a protein of the disclosure may also be generated by screening expression libraries encoding immunoglobulin genes, or portions thereof, expressed in bacteria with peptides produced from nucleic acid molecules of the present disclosure. For example, complete Fab fragments, VH regions and FV regions can be expressed in bacteria using phage expression libraries (See for example Ward et al., Nature 341, 544-546: (1989); Huse et al., Science 246, 1275-1281 (1989); and McCafferty et al. Nature 348, 552-554 (1990)).

In another embodiment of the disclosure, the binding proteins are apatmers, optionally nucleic acid (DNA or RNA) derived or peptide derived aptamers.

### (D) Modulators of ESC/iPSC Alternative Splicing

The present disclosure also relates to alternative splicing modulators to promote, enhance or maintain pluripotency.

In one embodiment, the modulators regulate FOXP1 alternative splicing. In one aspect of the invention, the present disclosure relates to FOXP1 “exon 18b splicing modulators”. In one embodiment, an “exon 18b splicing modulator” is a stimulator of FOXP1 exon 18b inclusion. In another embodiment, an exon 18b splicing modulator is a repressor of FOXP1 exon 18b inclusion.

The term “stimulator of exon 18b inclusion” as used herein includes all substances that can stimulate the inclusion of FOXP1 exon 18b or homologs thereof and includes, without limitation, modified or unmodified nucleic acids, peptides, polypeptides, proteins or fragments thereof, small molecule activators, chemical compounds, antibodies (and fragments thereof), aptamers, interfering RNA molecules such as siRNA, miRNA and shRNA and other substances that can stimulate the inclusion of FOXP1 exon 18b. The term “stimulator of exon 18b inclusion” also includes agents which act as cis-regulatory or trans-regulatory elements of FOXP1 exon 18b and agents which recognize intronic sequences located 300 bp upstream (SEQ ID NO: 36) or 600 bp downstream (SEQ ID NO: 37) of human FOXP1 exon 18 or 300 bp upstream (SEQ ID NO: 38) or 300 bp downstream (SEQ ID NO: 39) of human FOXP1 exon 18b.

In some embodiments of the present disclosure, the splicing modulator is MBNL1 (also known as Muscleblind-like). While the term “MBNL1” is often used to here to human MBNL1, in the present disclosure, the term “MBNL1” refers to a protein encoded by the MBNL1 gene and encompasses MBNL1 from any species or source, optionally mammalian, such as human or mouse and isoforms and homologs thereof.

Optionally, the term MBNL1 relates to human MBNL1. A sequence corresponding to human MBNL1 is provided in GenBank under Accession No. AAH50535.1

Optionally, the term Mbnl1 relates to mouse Mbnl1. A sequence corresponding to mouse Mbnl1 is provided in GenBank under Accession No. AAH96600.1.

In some embodiments of the present disclosure, the splicing modulator is MBNL2 (also known as Muscleblind-like 2). While the term “MBNL2” is often used to here to human MBNL2, in the present disclosure, the term “MBNL2” refers to a protein encoded by the MBNL2 gene and encompasses MBNL2 from any species or source, optionally mammalian, such as human or mouse and isoforms and homologs thereof.

Optionally, the term MBNL2 relates to human MBNL2. A sequence corresponding to human MBNL2 is provided in GenBank under Accession No. AAI04040.1.

Optionally, the term Mbnl2 relates to mouse Mbnl2. A sequence corresponding to mouse Mbnl2 is provided in GenBank under Accession No. AAH75665.1.

In other embodiments of the present disclosure, the splicing modulator is TIA1 (also known as T Cell intracellular antigen-1). While the term “TIA1” is often used to here to human TIA1, in the present disclosure, the he term “TIA1” refers to a protein encoded by the TIA1 gene and encompasses TIA1 from any species or source, optionally mammalian, such as human or mouse and isoforms and homologs thereof.

Optionally, the term TIA1 relates to human TIA1. A sequence corresponding to human TIA1 is provided in GenBank under Accession No. AAH15944.1.

Optionally, the term TIA1 relates to mouse Tia1. A sequence corresponding to mouse Tia1 is provided in GenBank under Accession No. AAH23813.1.

In other embodiments of the present disclosure, the splicing modulator is TIAL1 (also known as TIA1 cytotoxic granule-associated RNA binding protein-like 1). While the term “TIAL1” is often used to here to human TIAL1, in the present disclosure, the he term “TIAL1” refers to a protein encoded by the TIAL1 gene and encompasses TIAL1 from any species or source, optionally mammalian, such as human or mouse and isoforms and homologs thereof.

Optionally, the term TIAL1 relates to human TIAL1. A sequence corresponding to human TIAL1 is provided in GenBank under Accession No. AAH30025.1.

Optionally, the term TIAL1 relates to mouse Tial1. A sequence corresponding to mouse Tial1 is provided in GenBank under Accession No. AAH10496.1.

In an embodiment, a “stimulator of exon 18b inclusion” is TIA1, TIAL1, an MBNL1 and/or MBNL2 antagonist or antisense or siRNA that decreases the expression of FOXP1.

An “MBNL1 and/or MBNL2 antagonist” is any factor that decreases the expression and/or function of MBNL1 and/or MBNL2. Optionally, an “MBNL1 and/or MBNL2 antagonist” is any factor that reduces and/or inhibits the activity of MBNL1 and/or MBNL2. Optionally, the activity of MBNL1 and/or MBNL2 includes the regulation of alternative splicing of FOXP1 and other ESC/iPSC-specific targets. In one embodiment, an “MBNL1 and/or MBNL2 antagonist” promotes inclusion of exon 18b in FOXP1 and/or exon 16b in Foxp1.

Examples of MBNL1 and/or MBNL2 antagonists include antibody or peptide or nucleic-acid derived aptamers to MBNL1 or MBNL2, antisense RNA or small interfering RNA that decrease expression of MBNL1 and/or MBNL2, or compounds that target MBNL1 and/or MBNL2, or the genes encoding MBNL1 and/or MBNL2. MBNL1 and MBNL2 antagonists are optionally used alone or in combination.

The term “repressor of exon 18b inclusion” as used herein includes all substances that can repress the inclusion of FOXP1 exon 18b or homologs thereof and includes, without limitation, modified or unmodified nucleic acids, peptides, polypeptides, proteins or fragments thereof, small molecule activators, chemical compounds, antibodies (and fragments thereof), aptamers, interfering RNA molecules such as siRNA, miRNA and shRNA, and other substances that can repress the inclusion of FOXP1 exon 18b. The term “repressor of exon 18b inclusion” also includes agents which act as cis-regulatory or trans-regulatory elements of FOXP1 exon 18b and agents which recognize intronic sequences located 300 bp upstream (SEQ ID NO: 36) or 300 bp downstream (SEQ ID NO: 37) of human FOXP1 exon 18 or 300 bp upstream (SEQ ID NO: 38) or 300 bp downstream (SEQ ID NO: 39) of human FOXP1 exon 18b.

In an embodiment of the present disclosure, a “repressor of exon 18b inclusion” is MBNL1, MBNL2, a TIA1 or TIAL1 antagonist, or antisense or siRNA that decreases expression of FOXP1-ES.

A “TIA1 or TIAL1 antagonist” is any factor that decreases the expression and/or function of TIA1 or TIAL1. Optionally, an “TIA1 or TIAL1 antagonist” is any factor that reduces and/or inhibits the activity of TIA1 or TIAL1. Optionally, the activity of TIA1 or TIAL1 includes the regulation of alternative splicing of FOXP1 and other ESC/iPSC-specific targets. In one embodiment, a “TIA1 or TIAL1 antagonist” represses inclusion of exon 18b in FOXP1 and/or exon 16b in Foxp1.

Examples of TIA1 or TIAL1 antagonists include antibody or peptide or nucleic-acid derived aptamers to TIA1 or TIAL1, antisense RNA or small interfering RNA that decrease expression of TIA1 or TIAL1, or compounds that target TIA1 or TIAL1 or the genes encoding TIA1 or TIAL1. TIA1 and TIAL1 antagonists are optionally used alone or in combination.

In one embodiment, the “exon 18b splicing modulator” is an antisense oligonucleotide to any one of SEQ ID NOs 36-39. In one aspect of the disclosure, the antisense oligonucleotides act to inhibit the binding of factors that stimulate or repress the inclusion of exon 18b. In another embodiment, antisense oligonucleotides are tethered to factors that stimulate or repress the inclusion of exon 18b.

Also contemplated in the disclosure are “exon 16b splicing modulators”.

The “exon 16b splicing modulator” can be a stimulator of Foxp1 exon 16b inclusion or a repressor of Foxp1 exon 16b inclusion.

The term “stimulator of exon 16b inclusion” or “repressor of exon 16b inclusion” as used herein includes all substances that can stimulate or repress the inclusion of mouse Foxp1 exon 16b and includes, without limitation, modified or unmodified nucleic acids, peptides, polypeptides, proteins or fragments thereof, small molecule activators, chemical compounds, antibodies (and fragments thereof), aptamers, interfering RNA molecules such as siRNA, miRNA and shRNA and other substances that can stimulation the inclusion of mouse Foxp1 exon 16b. The term “stimulator of exon 16b inclusion” includes agents which act as cis-regulatory or trans-regulatory elements of Foxp1 exon 16b and agents which recognize intronic sequences located 300 bp upstream (SEQ ID NO: 40) or 300 bp downstream (SEQ ID NO: 41) of mouse Foxp1 exon 16 or 300 bp upstream (SEQ ID NO: 42) or 300 bp downstream (SEQ ID NO: 43) of mouse Foxp1 exon 16b.

In one embodiment, the “exon 16b splicing modulator” is an antisense oligonucleotide to any one of SEQ ID NOs 40-43. In one aspect of the disclosure, the antisense oligonucleotides act to inhibit the binding of factors that stimulate or repress the inclusion of exon 16b. In another embodiment, antisense oligonucleotides are tethered to factors that stimulate or repress the inclusion of exon 16b.

### (E) Applications

The above nucleic acids, polypeptides, peptides, exon 18b splicing modulators and exon 16b splicing modulators of the disclosure can be used to promote the maintenance of the self-renewal and pluripotency properties of cells. The above nucleic acids, polypeptides peptides and exon 16b/18b splicing modulators of the disclosure can also be used to reprogram somatic cells into induced pluripotent stem cells.

In one aspect of the methods and uses provided in the present disclosure, the cells are from any species, optionally a mammalian species such as human or mouse.

### (i) Maintenance of Pluripotency

The above described nucleic acid, polypeptide and peptide molecules allow those skilled in the art to maintain the pluripotent state of stem cells.

The term “stem cell” as used herein refers to a cell that has the ability for self-renewal (the ability to go through numerous cycles of cell division while maintaining the undifferentiated state) and the capacity to differentiate into specialized cell types. The stem cells of the disclosure are optionally embryonic stem cells or induced pluripotent stem cells. In one embodiment, the cells are from any species, optionally a mammalian species such as human or mouse.

In one embodiment, the stem cell is a pluripotent stem cell. The term “pluripotent” as used herein refers to the ability of a cell to differentiate into one of many cell types. Embryonic stem cells and induced pluripotent stem cells are pluripotent stem cells.

The term “differentiation” or “differentiated” as used herein refers to the process by which a less specialized cell, such as a stem cell, becomes a more specialized cell type, such that it is committed to a specific lineage.

The terms “maintain the pluripotent state” or “maintain the undifferentiated state” of stem cells may include maintaining the pluripotency of stem cells, maintaining the self-renewal of stem cells and/or suppressing stem cell differentiation.

As used herein the term “cell culture” refers to one or more cells grown under controlled conditions and optionally includes a cell line. The term “cell line” refers to a plurality of cells that are the product of a single group of parent cells. According to the teaching of the present disclosure, cells may be cultured according to any method known in the art. Optionally, cells may be cultured in culture medium comprising conditioned medium, non-conditioned medium, or embryonic stem cell medium. Examples of suitable conditioned medium include IMDM, DMEM, or aMEM, conditioned with embryonic fibroblast cells (e.g. human embryonic fibroblast cells or mouse embryonic fibroblast cells), or equivalent medium. Examples of suitable non-conditioned medium include Iscove's Modified Delbecco's Medium (IMDM), DMEM, or aMEM, or equivalent medium. The culture medium may comprise serum (e.g. bovine serum, fetal bovine serum, calf bovine serum, horse serum, human serum, or an artificial serum substitute) or it may be serum free.

Accordingly, in one aspect of the disclosure, a method is provided for maintaining a homogeneous population of pluripotent stem cells. Optionally, a homogeneous population of pluripotent stem cells is a population of cells comprising at least: 50%, 60%, 70%, 80%, 90% or 100% pluripotent stem cells. Optionally, the pluripotent stem cells are induced pluripotent stem cells.

In one embodiment, a method of maintaining a homogeneous population of pluripotent stem cells comprises:
(1)(a) transfecting cells with a cDNA encoding FOXP1-ES,
(b) transfecting cells with a mRNA encoding FOXP1-ES,
(c) administering FOXP1-ES protein to cells,
(d) expressing cDNA encoding FOXP1-ES in cells,
(e) administering an exon 18b or exon 16b splicing modulator to cells,
(f) administering antisense RNA or small interfering RNA that decreases the expression of FOXP1 to cells, or
(g) administering genomic derived FOXP1 to cells and expressing the FOXP1-ES isoform in the cells; and
(2) culturing the cells.

In one embodiment of the method, the FOXP1-ES or FOXP1 is human FOXP1-ES or human FOXP1. In another embodiment, the FOXP1-ES or FOXP1 is mouse Foxp1-ES or mouse Foxp1. In a further embodiment, the cells are embryonic stem cells optionally human embryonic stem cells or mouse embryonic stem cells. In yet another embodiment, the cells are induced pluripotent stem cells.

In another embodiment of the disclosure, a method of maintaining a homogenous population of stem cells comprises decreasing the expression or function of FOXP1. Decreasing the expression of FOXP1 may be accomplished by any method known in the art including, but not limited to the use of genetic modifications, including knockdown by antisense RNA, small interfering RNAs, microRNAs, antibodies or binding proteins, aptamers, retroviral agents and small molecule compounds or toxins.

In another aspect of the disclosure, a method is provided for suppressing the differentiation of stem cells. The stem cells may be embryonic stem cells, optionally embryonic stem cells of any species such as human embryonic stem cells, mouse embryonic stem cells or induced pluripotent stem cells. The term “suppressing the differentiation” refers to suppressing the differentiation or maturation of a stem cell to later lineage cell stages.

In one embodiment, a method for suppressing the differentiation of stem cells comprises:
(1)(a) transfecting cells with a cDNA encoding FOXP1-ES,
(b) transfecting cells with a mRNA encoding FOXP1-ES,
(c) administering FOXP1-ES protein to cells,
(d) expressing cDNA encoding FOXP1-ES in cells,
(e) administering an exon 18b or exon 16b splicing modulator to cells,
(f) administering antisense RNA or small interfering RNA that decreases the expression of FOXP1 to cells, or
(g) administering genomic derived FOXP1 to cells and expressing the FOXP1-ES isoform in the cells; and
(2) culturing the cells.

In one embodiment of the method, the FOXP1-ES or FOXP1 is human FOXP1-ES or human FOXP1. In another embodiment, the FOXP1-ES or FOXP1 is mouse Foxp1-ES or mouse Foxp1. In a further embodiment, the cells are embryonic stem cells of any species, optionally human embryonic stem cells or mouse embryonic stem cells.

In another embodiment of the disclosure, a method for suppressing the differentiation of stem cells comprises decreasing the expression or function of FOXP1. Decreasing the expression of FOXP1 may be accomplished by any method known in the art including, but not limited to the use of genetic modifications, including knockdown by antisense RNA, small interfering RNAs, microRNAs, antibodies or binding proteins, retroviral agents and small molecule compounds or toxins.

In another aspect of the disclosure, a method is provided for producing a population of differentiated cells.

In one embodiment, a method for producing a population of differentiated cells comprises:
(1)(a) transfecting stem cells with a cDNA encoding FOXP1,
(b) transfecting stem cells with a mDNA encoding FOXP1,
(c) administering FOXP1 protein to stem cells,
(d) inhibiting the expression of FOXP1-ES in stem cells,
(e) administering an exon 18b or exon 16b splicing modulator to cells,
(f) administering antisense RNA or small interfering RNA that decreases the expression of FOXP1-ES to cells, or
(g) administering genomic derived FOXP1 to cells and expressing the FOXP1 isoform in the cells;
and (2) culturing the cells.

In one embodiment of the method, the FOXP1-ES or FOXP1 is human FOXP1-ES or human FOXP1. In another embodiment, the FOXP1-ES or FOXP1 is mouse Foxp1-ES or mouse Foxp1. In a further embodiment, the cells are embryonic stem cells of any species, optionally human embryonic stem cells, mouse embryonic stem cells or induced pluripotent stem cells.

In another embodiment of the disclosure, a method for producing a population of differentiated cells comprises decreasing the expression or function of FOXP1-ES. Decreasing the expression of FOXP1-ES may be accomplished by any method known in the art including, but not limited to the use of genetic modifications, including knockdown by antisense RNA, small interfering RNA, microRNA, antibodies or binding proteins, aptamers, retroviral agents and small molecule compounds or toxins.

(ii) Reprogramming Somatic Cells into Induced Pluripotent Stem Cells

The above described nucleic acid, polypeptide and peptide molecules also allow those skilled in the art to reprogram somatic cells into induced pluripotent stem cells. The somatic cells may be cells from any species, optionally a mammalian species such as human or mouse.

The term “induced pluripotent stem cell” refers to a pluripotent stem cell that has been artificially derived from a non-pluripotent cell.

Accordingly, in an embodiment of the disclosure, the inventors provide a method of reprogramming somatic cells into pluripotent stem cells comprising:
(1)(a) transfecting somatic cells with a cDNA encoding FOXP1-ES,
(b) transfecting somatic cells with a mRNA encoding FOX-P1-ES,
(c) expressing cDNA encoding FOXP1-ES in somatic cells,
(d) administering FOXP1-ES protein to a culture of somatic cells,
(e) administering an exon 18b splicing modulator to cells,
(f) administering antisense RNA or small interfering RNA that decreases the expression of FOXP1 to cells, or
(g) administering genomic derived FOXP1 to cells and expressing the FOXP1-ES isoform in the cells; and
(2) culturing under conditions that allow reprogramming of the somatic cells into induced pluripotent stem cells.

In one embodiment, the FOXP1-ES is human FOXP1-ES. In another embodiment, the FOXP1-ES is mouse Foxp1-ES. In a further embodiment, the somatic cells are somatic cells from any species, optionally human somatic cells or mouse somatic cells.

Optionally, the method comprises reprogramming embryonic fibroblasts, optionally fibroblast from any species such as human embryonic fibroblasts or mouse embryonic fibroblasts into induced pluripotent stem cells.

In some embodiments, the induced pluripotent stem cells exhibit morphology traits similar to the embryonic stem cells described herein such as increased expression of SSEA-1.

The present disclosure further provides isolated induced pluripotent stem cells generated by the method described herein and cells differentiated therefrom. In another aspect, the disclosure provides use of the cells described herein as a source of induced pluripotent stem cells or differentiated cells therefrom.

(iii) Modulation of FOXP1-ES Expression

In another embodiment, the disclosure provides a method of modulating the expression of FOXP1-ES. In one embodiment, the disclosure provides a method of modulating the expression of FOXP1-ES in a cell comprising administering an exon 18b splicing modulator to the cell.

Also contemplated in the disclosure are methods of modulating the expression of mouse Foxp1-ES in a cell comprising administering an exon 16b splicing modulator to the cell.

The term “administering to a cell” includes, without limitation transfecting a cell with nucleic acid corresponding to an exon 18b or 16b splicing modulator, overexpressing an exon 18b or 16b splicing modulator in a cell and exposing a cell to an exon 18b or 16b splicing modulator.

The present disclosure also relates to the use of the exon 18b or 16b splicing modulators to maintain stem cells, for example embryonic stem cells, optionally human or mouse embryonic stem cells, in a self-renewing and pluripotent state. The disclosure further relates to the use of exon 18b or 16b splicing modulators for the production of self-renewing, pluripotent stem cells (for example, induced pluripotent cells).

(iii) Markers of Differentiation

The present disclosure also relates to the use of the nucleic acids, polypeptides and peptides described herein as markers of differentiation or the differentiation state of an individual cell or a population of cells. In one embodiment, the nucleic acids, polypeptides and peptides described herein are for use as markers of cells that have been reprogrammed into pluripotent stem cells.

In one embodiment, the expression of higher levels of FOXP1-ES and/or lower levels of FOXP1 is correlated with pluripotency. In one embodiment, the expression of FOXP1-ES and/or suppression of FOXP1 in a cell indicates that the cell is a pluripotent cell such as an embryonic stem cell or an induced pluripotent stem cell.

In another embodiment, the expression of lower levels of MBNL1 and/or MBNL2 is correlated with pluripotency. In one embodiment, the suppression of MBNL1 and/or MBNL2 in a cell indicates that the cell is a pluripotent cell such as an embryonic stem cell or an induced pluripotent stem cell.

In a further embodiment, the expression of higher levels of FOXP1 and/or lower levels of FOXP1-ES is correlated with cell differentiation. In one embodiment, the expression of FOXP1 and/or suppression of FOXP1-ES in a cell indicates that the cell is a differentiated cell. In yet another embodiment, expression of MBNL1 and/or MBNL2 indicates that the cell is a differentiated cell.

Accordingly, the disclosure relates to a method of assessing the pluripotency of a cell population comprising detecting the level of expression of FOXP1-ES in a sample of cells from the population, wherein an increase in the level of FOXP1-ES compared to a reference level in the sample of cells indicates the pluripotency of the cell population.

The disclosure also relates to a method of assessing the pluripotency of a cell population over time comprising

(a) detecting the level of expression of FOXP1-ES in a sample of cells from the population at a first time point,
(b) detecting the level of expression of FOXP1-ES in a sample of cells from the population at a second time point,
wherein an increase in the level of FOXP1-ES in the sample of cells at the second time point compared to the first time point indicates increased pluripotency of the cell population.

Optionally, the reference level is the expression level of FOXP1-ES in a differentiated or partially differentiated cell. Optionally a 5, 10, 15, 25, 50, 75, 100 or 200% increase in the level of FOXP1-ES in a sample of cells compared to a reference level indicates that the cell population comprises pluripotent cells.

The disclosure further relates to a method of assessing the pluripotency of a cell population comprising detecting the level of expression of FOXP1 in a sample of cells from the population, wherein a decrease in the level of FOXP1 compared to a reference level in the sample of cells indicates the pluripotency of the cell population.

The disclosure also relates to a method of assessing the pluripotency of a cell population over time comprising

(a) detecting the level of expression of FOXP1 in a sample of cells from the population at a first time point,
(b) detecting the level of expression of FOXP1 in a sample of cells from the population at a second time point,
wherein a decrease in the level of FOXP1 in the sample of cells at the second time point compared to the first time point indicates increased pluripotency of the cell population.

Optionally, the reference level is the expression level of FOXP1 in a differentiated or partially differentiated cell. Optionally a 5, 10, 15, 25, 50, 75, 100 or 200% decrease in the level of FOXP1 in a sample of cells compared to a reference level indicates that the cell population comprises pluripotent cells.

The disclosure further relates to a method of assessing the pluripotency of a cell population comprising detecting the level of expression of MBNL1 and/or MBNL2 in a sample of cells from the population, wherein a decrease in the level of MBNL1 and/or MBNL2 compared to a reference level in the sample of cells indicates the pluripotency of the cell population.

The disclosure also relates to a method of assessing the pluripotency of a cell population over time comprising

(a) detecting the level of expression of MBNL1 and/or MBNL2 in a sample of cells from the population at a first time point,
(b) detecting the level of expression of MBNL1 and/or MBNL2 in a sample of cells from the population at a second time point,
wherein a decrease in the level of MBNL1 and/or MBNL2 in the sample of cells at the second time point compared to the first time point indicates increased pluripotency of the cell population.

Optionally, the reference level is the expression level of MBNL1 and/or MBNL2 in a differentiated or partially differentiated cell. Optionally a 5, 10, 15, 25, 50, 75, 100 or 200% decrease in the level of MBNL1 and/or MBNL2 in a sample of cells compared to a reference level indicates that the cell population comprises pluripotent cells.

The disclosure relates to a method of assessing the differentiation state of a cell population comprising detecting the level of expression of FOXP1-ES in a sample of cells from the population, wherein a decrease in the level of FOXP1-ES compared to a reference level in the sample of cells indicates increased differentiation of the cell population.

The disclosure also relate to a method of assessing the differentiation state of a cell population over time comprising

(a) detecting the level of expression of FOXP1-ES in a sample of cells from the population at a first time point,
(b) detecting the level of expression of FOXP1-ES in a sample of cells from the population at a second time point,
wherein a decrease in the level of FOXP1-ES in the sample of cells at the second time point compared to the first time point indicates increased differentiation of the cell population.

Optionally, the reference level is the expression level of FOXP1-ES in a pluripotent cell. Optionally a 5, 10, 15, 25, 50, 75, 100 or 200% decrease in the level of FOXP1-ES in a sample of cells compared to a reference level indicates that the cell population comprises differentiated cells.

The disclosure also relates to a method of assessing the differentiation state of a cell population comprising detecting the level of expression of FOXP1 in a sample of cells from the population, wherein an increase in the level of FOXP1 compared to a reference level in the sample of cells indicates increased differentiation of the cell population.

The disclosure also relates to a method of assessing the differentiation state of a cell population over time comprising

(a) detecting the level of expression of FOXP1 in a sample of cells from the population at a first time point,
(b) detecting the level of expression of FOXP1 in a sample of cells from the population at a second time point,
wherein an increase in the level of FOXP1 in the sample of cells at the second time point compared to the first time point indicates increased differentiation of the cell population.

Optionally, the reference level is the expression level of FOXP1 in a pluripotent cell. Optionally a 5, 10, 15, 25, 50, 75, 100 or 200% increase in the level of FOXP1 in a sample of cells compared to a reference level indicates that the cell population comprises differentiated cells.

The disclosure further relates to a method of assessing the differentiation state of a cell population comprising detecting the level of expression of MBNL1 and/or MBNL2 in a sample of cells from the population, wherein an increase in the level of MBNL1 and/or MBNL2 compared to a reference level in the sample of cells increased differentiation of the cell population.

The disclosure also relates to a method of assessing the pluripotency of a cell population over time comprising

(a) detecting the level of expression of MBNL1 and/or MBNL2 in a sample of cells from the population at a first time point,
(b) detecting the level of expression of MBNL1 and/or MBNL2 in a sample of cells from the population at a second time point,
wherein an increase in the level of MBNL1 and/or MBNL2 in the sample of cells at the second time point compared to the first time point indicates increased differentiation of the cell population.

Optionally, the reference level is the expression level of MBNL1 and/or MBNL2 in a pluripotent cell. Optionally a 5, 10, 15, 25, 50, 75, 100 or 200% increase in the level of MBNL1 and/or MBNL2 in a sample of cells compared to a reference level indicates that the cell population comprises differentiated cells.

As used herein, “expression” relates to the expression of gene and/or proteins in a cell. Methods of measuring the expressions levels of genes and proteins in a cell are well known in the art.

### (F) Modulation of ESC/iPSc-Specific Alternative Splicing and Reprogramming

The present disclosure includes evidence that alternative splicing modulators such as MBNL1/Mbnl1 and MBNL2/Mbnl2 regulate a large number ESC/iPSC-specific alternative splicing events

As such, the present disclosure relates also to the use of alternative splicing modulators such as MBNL1, MBNL2, TIA1, and TIAL1 and antagonists thereof, to regulate ESC/iPSC-specific alternative splicing events through FOXP1 exon 18b as well as targets other than FOXP1 exon 18b.

Accordingly, the present disclosure also relates to the use of modulators of ESC/iPSc-specific alternative splicing and reprogramming (for example, MBNL1 antagonists, MBNL2 antagonists, TIA1 and/or TIAL1, alone or in combination) to maintain or enhance pluripotency.

In one embodiment, the application relates to the use of a MBNL1 antagonist and a MBNL2 antagonist to maintain or enhance pluripotency. In another embodiment, the application relates to the use of a TIA1, and TIAL1 to maintain or enhance pluripotency. The phrase “maintaining or enhancing pluripotency” includes producing pluripotent stem cells, maintaining a homogeneous population of pluripotent stem cells, suppressing stem cell differentiation or reprogramming somatic cells into pluripotent stem cells.

Optionally, “maintaining or enhancing pluripotency” as used herein includes, without limitation, increasing the efficiency and/or kinetics of producing pluripotent stem cells, maintaining a homogeneous population of pluripotent stem cells, suppressing stem cell differentiation or reprogramming somatic cells into pluripotent stem cells. “Maintaining or enhancing pluripotency” as used herein also includes, without limitation, increasing the homogeneity of a population of pluripotent stem cells.

“Increasing the efficiency and/or kinetics” refers to increasing the efficiency and/or kinetics of a process by at least 5, 10, 25, 75, 100, 150 or 200%.

In other embodiments, modulators of ESC/iPSC-specific alternative splicing and reprogramming (for example, MBNL1 antagonists, MBNL2 antagonists, TIA1, and TIAL1) are used to accelerate or enhance the production of pluripotent stem cells, suppress stem cell differentiation, or reprogram somatic cells into pluripotent stem cells.

The present disclosure also relates to the use of MBNL1 and/or MBNL2, to promote differentiation or produce a population of differentiated cells. The present disclosure also relates to the use of a TIA1 antagonist and/or a TIAL1 antagonist to promote differentiation or produce a population of differentiated cells.

The following non-limiting examples are illustrative of the present disclosure:

### EXAMPLES

### Example 1

### Identification and Characterization of FOXP1 Splice Variants

An Embryonic Stem Cell-Specific Splice Variant from the FOXP1 Gene

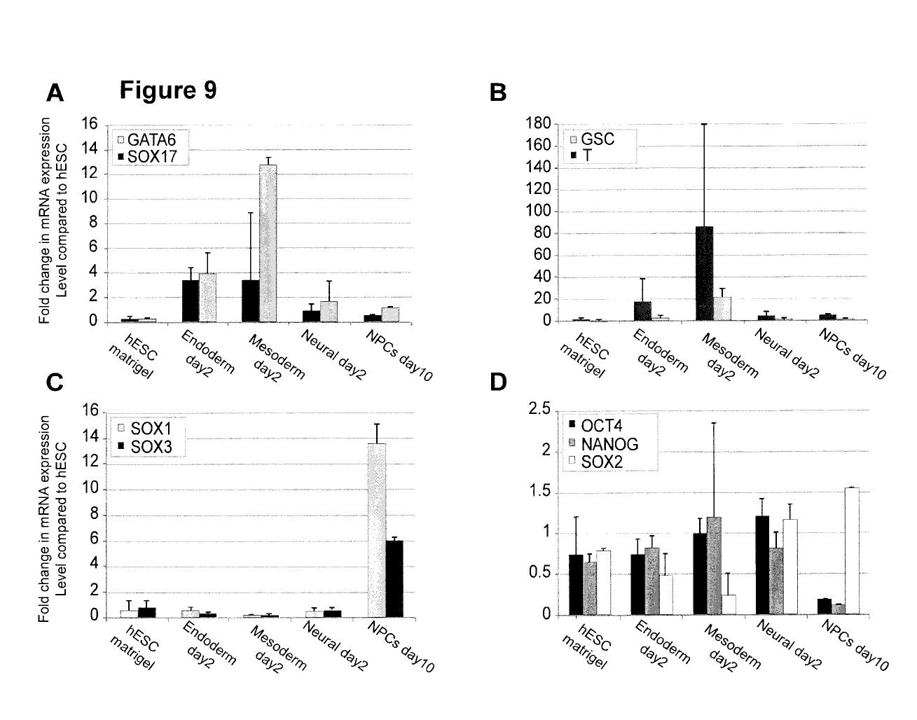

In order to identify AS events that may function in the control of stem cell pluripotency, microarray profiling was used to compare patterns of alternative splicing (AS) in undifferentiated and differentiated H9 human embryonic stem cells (hESCs; FIG. 9). Whereas few AS changes were detected between H9 hESCs and the lineage-specified samples collected at day 2, the microarray data predicted that ˜165 (2.85%) of the profiled exons undergo inclusion level changes of >15% between H9 hESCs and neural lineage-specified cells collected at day 10. Genes containing these predicted AS changes are not significantly enriched in specific Gene Ontology (GO) terms but are represented by diverse functional categories. In this study, the focus was on a previously unidentified AS change detected in transcripts from the FOXP1 gene.

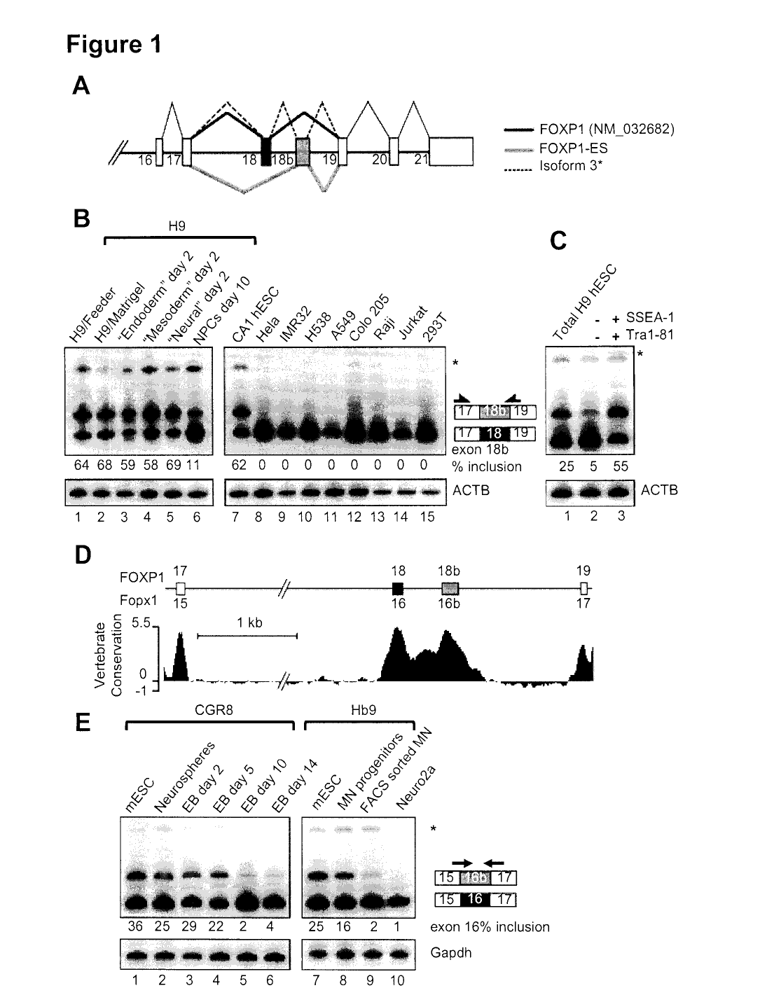

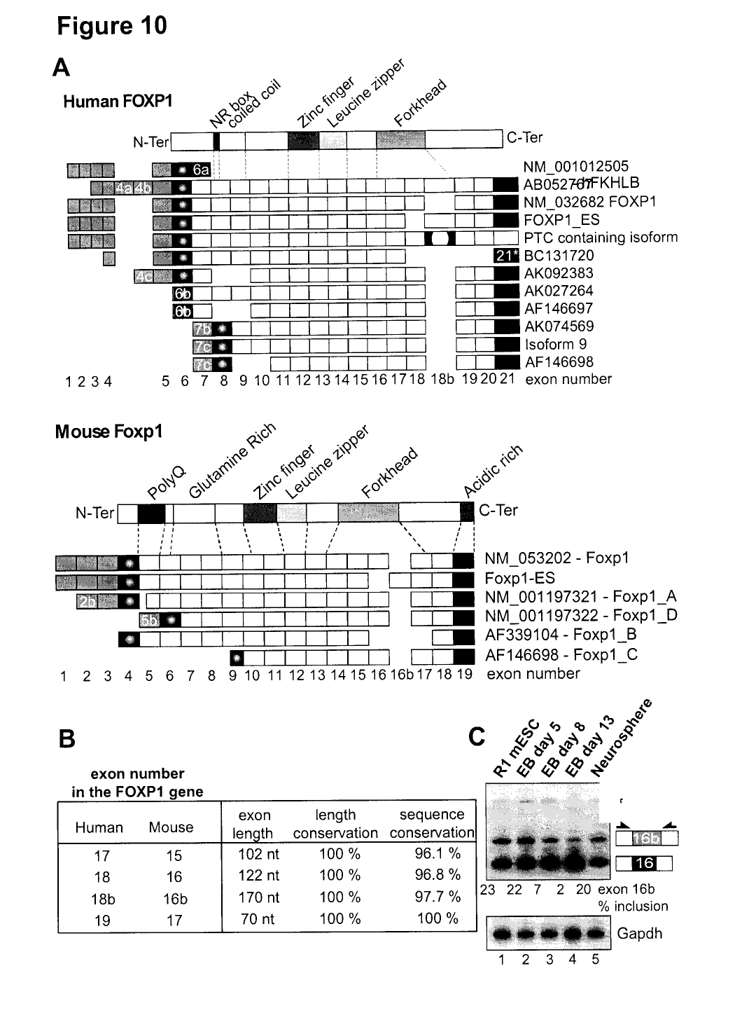

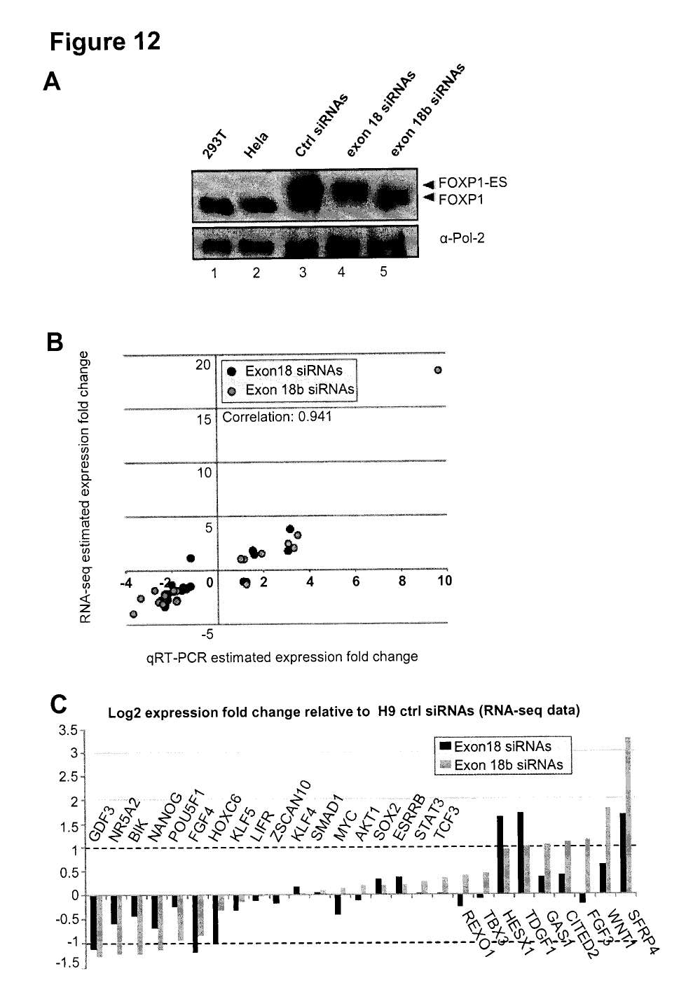

Microarray analysis indicated that human FOXP1 exon 18 has increased inclusion in day 10 neural progenitor-enriched cells (˜96% inclusion) compared to undifferentiated H9 hESCs (˜79% inclusion). RT-PCR assays using primers specific for FOXP1 exons 17 and 19 confirmed this prediction, and also detected two unexpected additional bands that are ˜50 nt and ˜170 nt longer than the transcripts containing exon 18 (FIG. 1A, NM_{—}032682). Sequencing of the +50 nt band revealed the inclusion of a previously uncharacterized exon in FOXP1 transcripts in place of exon 18, referred to as exon 18b. Sequencing of the +170 nt band revealed that it contains both exons 18 and 18b (indicated by an asterisk in FIG. 1). Consistent with low or undetectable (see below) expression of this isoform, simultaneous inclusion of exons 18 and 18b introduces a premature termination codon 121 nt upstream of exon 18b that likely elicits nonsense mediated mRNA decay. Inclusion of exon 18b instead of exon 18 preserves the FOXP1 open reading frame and produces a FOXP1 isoform with a modified C-terminal amino acid sequence (FIG. 10A). Exon 18b displays high levels of inclusion in undifferentiated H9 hESCs (>64%, FIG. 1B, lanes 1 and 2) and in H9 cells two days after differentiation induction (>58%, FIG. 1B, lanes 3 to 5), relative to the neural lineage-enriched cell population at day 10 (11%, FIG. 1B, lane 6). This observation is consistent with the relatively high proportion of undifferentiated H9 hESCs expressing pluripotency markers at day 2 compared to day 10 post induction of differentiation (FIG. 9D). Next RT-PCR assays were used to examine the regulation of exon 18 and 18b in another hESC line, CA1 (FIG. 1B, lane 7), and in a panel of diverse human cell lines derived from partially or fully differentiated cells (FIG. 1B lanes 8-15; refer to legend). Exon 18b is specifically included at similar levels (62%) in undifferentiated CA1 hESCs as found in H9 hESCs (64%), whereas exon 18 is included at a reduced level in CA1 hESCs and is the only exon detected in differentiated cell lines. Consistent with these results, western blotting experiments demonstrated that the exon 18b-containing isoform is more highly expressed at the protein level than is FOXP1 in hESCs, and also that it is not expressed in differentiated cell lines (FIG. 12A). These results demonstrate that FOXP1 exon 18b undergoes a switch from efficient inclusion in hESCs to almost complete skipping in differentiated hESCs and in cells derived from a variety of other differentiated sources.

To establish whether exon 18b is specifically included in self-renewing, pluripotent hESCs, RT-PCR assays were performed to measure the inclusion levels of exon 18 and 18b in flow cytometry-sorted H9 cells that express TRA1-81 and SSEA-3, two cell surface pluripotency markers (FIG. 1C). A partially differentiated population of H9 hESCs was used in this experiment, which displayed reduced levels of exon 18b inclusion relative to the levels of exon 18b inclusion detected in undifferentiated H9 hESCs (compare FIG. 1C lane 1 with lanes 1 and 2 in FIG. 1B). However, transcripts including exon 18b were highly enriched in sorted cells expressing TRA1-81 and SSEA-3, (FIG. 1C, lane 3), whereas only minor levels of exon 18b inclusion were detected in the TRA1-81/SSEA-3 negative H9 hESC population (FIG. 1C, lane 2). These results support the conclusion that inclusion of FOXP1 exon 18b is specific to self-renewing, pluripotent hESCs. In the sections that follow transcripts including exon 18b are referred to as the “FOXP1-ES” splice variant, and transcripts containing exon 18 are called the “FOXP1” variant.

### Evolutionary Conservation of FOXP1-ES Regulation

The evolutionary conservation of FOXP1 exons 18 and 18b as well as their regulated AS patterns were examined. A multispecies genome alignment encompassing human FOXP1 exons 17 to 19 and the orthologous sequences from 46 vertebrate species reveals that exons 18 and 18b are located within a ˜1000 nt genomic region that is highly conserved (PhastCons Mean 0.959, variance 0.029) across the analyzed species (FIG. 1D). This conserved region encompasses the 373 nt intron separating exons 18 and 18b and extends ˜120 nts into the intron upstream of exon 18 and ˜205 nts into the intron downstream of exon 18b. This observation shows that the intronic sequences surrounding exons 18 and 18b likely govern conserved patterns of regulation in diverse vertebrate species.

To determine whether differential AS of FOXP1 exons 18 and 18b between hESCs and differentiated cells is conserved, RT-PCR assays were used to analyze the regulation of the orthologous exons (exons 16 and 16b; FIG. S2B) in mouse Foxp1 transcripts (FIG. 1D). The AS levels of exons 16 and 16b were analyzed in three undifferentiated mouse (m)ESCs lines, CGR8, Hb9 and R1, and in RNA samples collected following induction of differentiation of these mESCs into different lineages (FIGS. 1E and 10C). Similar to the results for human exon 18b, exon 16b displays the highest levels of inclusion in undifferentiated mESCs (FIG. 1E, lanes 1 and 7; FIG. 10C, lane 1) but its inclusion level progressively decreases when CGR8- or R1-derived embryoid bodies (EBs) are induced to form cardiomyocytes over a 14 day period (FIG. 1E, lanes 3 to 6; FIG. S2C, lanes 2 to 4). Furthermore, exon 16b displays decreased levels of inclusion in day 14 CGR8- or R1-derived neural and glial progenitor-enriched neurospheres (FIG. 1E, lane 2; FIG. 10C, lane 5), and when Hb9 mESCs are induced to form motor neuron (MN) precursors (FIG. 1E, lane 8). It is almost entirely skipped in sorted, differentiated MNs and in the neuroblastoma cell line Neuro2A (FIG. 1E, lanes 9 and 10). Also similar to the results for human exon 18, mouse exon 16 also displays inclusion in all of the samples, but reduced levels of inclusion relative to exon 16b in undifferentiated compared to differentiated mESCs. Thus, consistent with the high degree of sequence conservation of exons 18b/16b and 18/16 and the surrounding intronic regions, these exons display conserved patterns of regulation between ESCs and differentiated cells.

### FOXP1 Genomic Co-Ordinates

The following table lists the genomic co-ordinates of exons 17, 18, 18b and 19 of human FOX P1.

TABLE 1
Human FOX P1 (hg assembly, - strand)

| | | |
|---|---|---|
| exon 17 | chr3 | 71026092-71026193 |
| Alternative exon 18 | chr3 | 71021706-71021827 |
| ES specific exon 18b | chr3 | 71021162-71021331 |
| exon 19 | chr3 | 71019887-71019956 |

The following table lists the genomic co-ordinates of exons 15, 16, 16b and 17 of mouse Foxp1.

TABLE 2
Mouse Foxp1 (mm9 assembly, - strand)

| | | |
|---|---|---|
| exon 15 | chr6 | 98894613-98894714 |
| Alternative exon 16 | chr6 | 98891530-98891651 |
| ES specific exon 16b | chr6 | 98890989-98891158 |
| exon 17 | chr6 | 98889713-98889782 |

FOXP1 and FOXP1-ES have Distinct DNA Binding Specificities

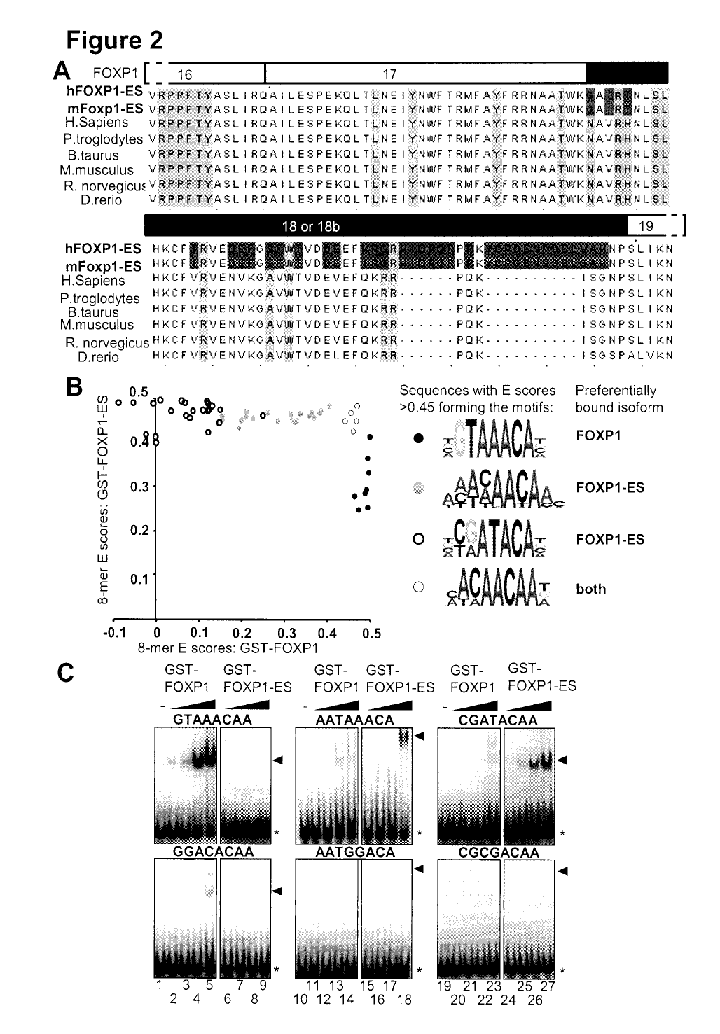

The coding sequence of the DNA-binding Forkhead domain of human FOXP1 overlaps exons 16 to 19. It was investigated whether differential AS of exons 18 and 18b affects the DNA binding properties of FOXP1. The Forkhead domains of FOXP family members are highly homologous and bind the same canonical consensus motif GTAAACA as monomers, homo- and/or heterodimers (Koh et al., 2009; Li et al., 2004). A co-crystal structure of FOXP2 bound to DNA (Stroud et al., 2006) reveals that residues forming direct contacts with DNA are conserved in FOXP1. Interestingly, the inclusion of exon 18b instead of exon 18 is predicted to substitute a total of 35 residues that overlap with the Forkhead domain. Four highly conserved amino acid residues (Asn510, His514, Ala531 and Arg543) in FOXP1 shown to directly contact DNA in FOXP2 are among those substituted (with Gly510, Tyr514, Ser 531 and Gly543) by exon 18b splicing. None of the sequence changes resulting from exon 18b splicing are expected to alter the overall secondary structure of the FOXP1 forkhead domain, nor its capacity to multimerize (amino acids involved in dimer formation are indicated by black dots in FIG. 2A). However, the amino acid substitutions involving Asn510 and His514, which form critical hydrogen bonds with the adenine-thymine (A-T) basepair at the fourth position in the canonical FOXP DNA binding site (underlined in: GTAAACA) are expected to affect binding affinity and/or specificity.

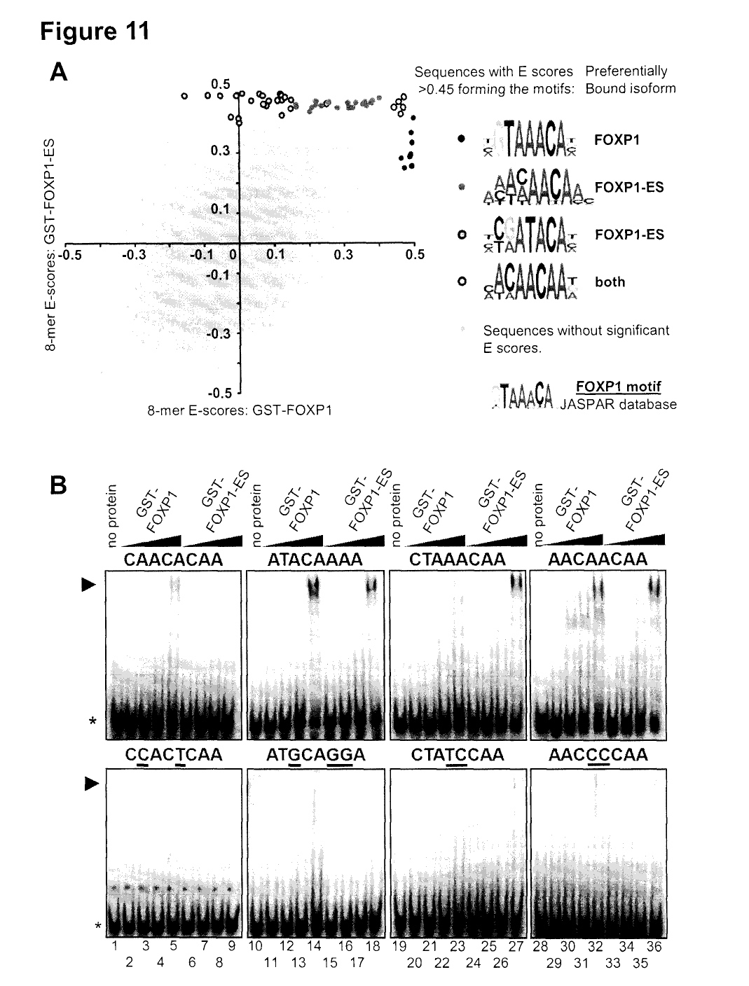

To investigate the consequence of exon 18b splicing on DNA binding, protein binding microarray experiments were performed (PBM: Berger et al., 2008; Berger et al., 2006) to define 8-mer sequences that are preferentially bound by FOXP1 and FOXP1-ES forkhead domains fused to glutathione S transferase (GST). GST-FOXP1 and GST-FOXP1-ES preferentially recognize distinct DNA binding motifs (FIGS. 2B and 11A). The canonical binding motif GTAAACAA was represented by the majority of the highest-scoring FOXP1-associated sequences. In contrast, the majority of the highest-scoring probe sequences preferentially bound by the FOXP1-ES forkhead domain contained motifs consisting of CGATACAA or closely related sequences. Other high-scoring probe sequences preferentially bound by FOXP1-ES contain specific C/A-rich motifs and other C/A-rich motifs are bound by both proteins (FIGS. 2B and 11A).

The PBM-defined binding preferences of the FOXP1 and FOXP1-ES forkhead domains were confirmed by gel mobility shift assays using the GST-forkhead fusion proteins and radiolabeled dsDNA probes (FIGS. 2C and 11B). For example, GST-FOXP1-ES, relative to GST-FOXP1, preferentially bound probes containing the sequences AATAAACA and CGATACAA (FIG. 2B). GST-FOXP1, relative to GST-FOXP1-ES, preferentially bound a probe containing the consensus GTAAACA. Further confirming binding specificity, GST-FOXP1 and GST-FOXP1-ES did not appreciably alter the mobility of probes that contained mutant versions of each of the analyzed PBM-derived binding sites (FIGS. 2C and 11B; mutant positions underlined).

These results show that hESC-specific inclusion of exon 18b changes the DNA binding specificity of FOXP1. Substitution of residues Asp510 and His514 as a consequence of exon 18b splicing affects the recognition of the A-T base pair at the fourth position of the FOXP1 consensus binding site. FOXP1-ES binds a T-A base pair at this position in a subset of the preferentially bound PBM sequences. Additional amino acid substitutions in FOXP1-ES affecting conserved residues at the DNA binding interface account for the other changes in the DNA binding properties of this splice isoform, including its preferential binding to specific C/A-rich motifs. In addition to a change in sequence preference, the combined results from the PBM experiments and gel mobility shift assays reveal that GST-FOXP1-ES can bind to a broader spectrum of sequences than does GST-FOXP1, although with apparent reduced affinity since at a similar concentration range of protein, GST-FOXP1-ES bound less efficiently to many of its highest scoring PBM sequences than did GST-FOXP1 (FIGS. 2 and 11A). These results show that the AS-mediated switch in FOXP1 transcripts directs different FOXP1-mediated gene expression programs.

FOXP1 and FOXP1-ES Regulate Distinct Programs of Gene Expression in hESCs

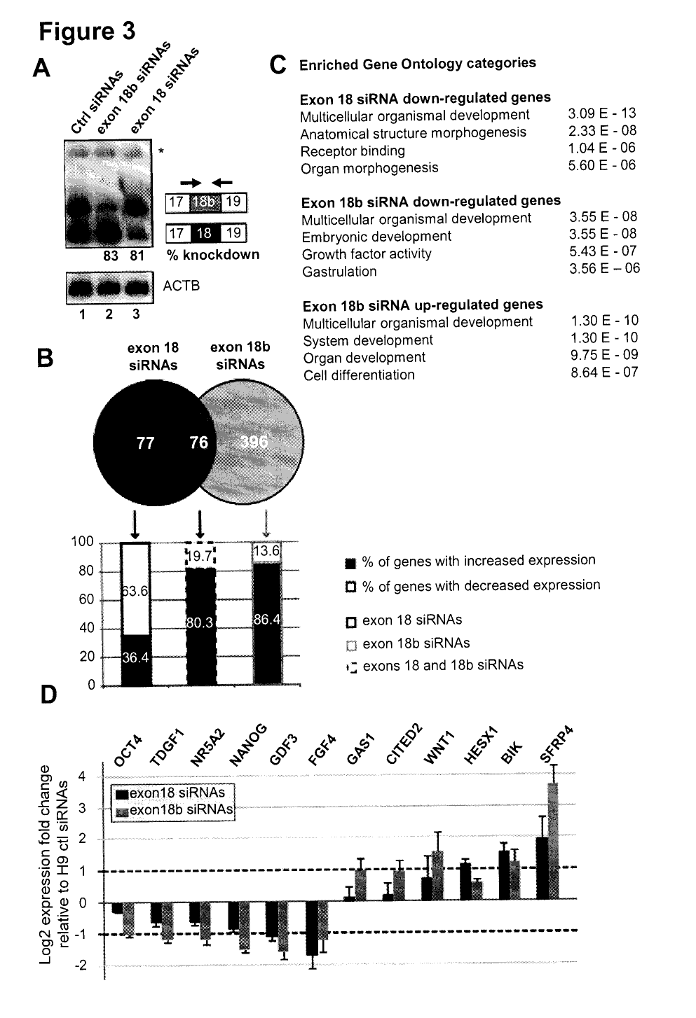

To show that FOXP1 and FOXP1-ES control the expression of different sets of genes, siRNA knockdowns of each isoform in undifferentiated H9 cells were performed followed by RNA-Seq profiling. H9 hESCs were transfected with custom siRNA pools targeting exons 18 or 18b. Relative to a control siRNA pool (FIG. 3A, lane 1), each isoform-targeting pool resulted in specific and efficient (>80%) knockdown of only the expected target transcripts, as monitored by RT-PCR (FIG. 3A, lanes 2 and 3), and supported by Western blotting experiments (FIG. 12A, lanes 4 and 5). RNA-Seq reads generated from polyA+ RNA from the control and each knockdown sample were mapped to a set of RefSeq cDNAs to establish the counts of unique-mapping reads per kb per million mapped sequenced reads (RPKM; (Mortazavi et al., 2008)). Genes with expression differences of at least 2-fold were selected for further analysis. Knockdown of FOXP1 resulted in changes in mRNA expression levels for 153 genes, whereas knockdown of FOXP1-ES had a more dramatic effect, resulting in altered mRNA expression levels of 472 genes, 76 of which overlap those affected by knockdown of FOXP1 (FIG. 3B). Confirming the accuracy of these observations, qRT-PCR measurements for a representative set of 19 genes with estimated expression changes ranging from 2 fold to 20 fold agreed well with the RNA-Seq-derived estimates for expression changes (r=0.941; FIG. 12B; see below).

A significantly higher proportion of genes showed increased expression upon knockdown of FOXP1-ES compared to knockdown of FOXP1 (86% vs. 58.2%; p=1.63E-05, Chi-square test). Moreover, of the 76 genes affected in both knockdowns (FIG. 3B), 61 (80.3%) displayed increased expression when either isoform was knocked-down. These results show that in undifferentiated hESCs, FOXP1 and FOXP1-ES control distinct but overlapping sets of genes, with a substantially larger set of genes controlled by FOXP1-ES compared to FOXP1 in hESCs. Moreover, FOXP1-ES appears to predominantly act to suppress gene expression.

A Gene Ontology (GO) enrichment analysis of genes with decreased mRNA expression upon knockdown of FOXP1 or FOXP1-ES revealed significant enrichment of terms associated with early development (p <1.21E-05, FIG. 3C). Interestingly, a subset of genes with decreased expression upon FOXP1-ES knockdown were associated with hESC pluripotency maintenance (see below and FIG. 3D). Genes up-regulated upon knockdown of FOXP1 were not significantly enriched in any GO category, whereas genes up-regulated upon knockdown of FOXP1-ES were highly significantly enriched in GO annotations associated with development, transmembrane receptor activity and cell differentiation (p<2.24E-06, FIG. 3C).

Validation experiments using qRT-PCR confirmed that knockdown of FOXP1-ES in H9 hESCs results in a ˜2-fold or greater decrease in the levels of mRNA expression of the pluripotency genes OCT4, NANOG, NR5A2, GDF3 and TDGF1, and a ˜2-fold or greater increase in the expression of differentiation-associated genes including GAS1, HESX1, SFRP4 and WNT1 (FIG. 3D). The expression levels of several other genes that function in pluripotency maintenance and reprogramming, including KLF4, KLF5, SOX2, C-MYC, ZSCAN10, ESRRB, REXO1 and TBX3, displayed negligible or less pronounced changes in mRNA expression upon FOXP1-ES knockdown (FIG. 12C). This suggests that the decreased expression of OCT4, NANOG, NR5A2, GDF3 and TDGF1 is a specific consequence of reduced levels of FOXP1-ES rather than an indirect effect arising from induction of differentiation, which otherwise would be expected to have more general effects on the expression levels of pluripotency genes. Further consistent with the RNA-Seq analysis, knockdown of FOXP1 resulted in negligible (<1.5 fold) changes in the expression levels of these and many other FOXP1-ES regulated genes (FIGS. 3D and 12C).

These results show that the AS-mediated expression of FOXP1-ES in hESCs predominantly functions in the suppression of a large number of genes with important functions in cell differentiation and development. Moreover, FOXP1-ES is also involved in promoting the expression of a specific subset of genes associated with pluripotency.

### Direct Binding of FOXP1-ES and FOXP1 to Regulated Target Genes

Chromatin immunoprecipitation was performed followed by high-throughput sequencing (ChIP-Seq) to identify genes that are bound and potentially directly regulated by FOXP1-ES and FOXP1 in H9 ESCs. To avoid introducing isoform-specific signal bias in the ChIP-Seq experiments, an antibody was used to efficiently immunoprecipitate both FOXP1 isoforms. The ChIP-Seq data revealed >3400 significant peaks across the human genome that were specific to immunoprecipitation with the anti-FOXP1 antibody. These peaks correspond to in vivo sites of FOXP1 and FOXP1-ES occupancy (FIG. 2).

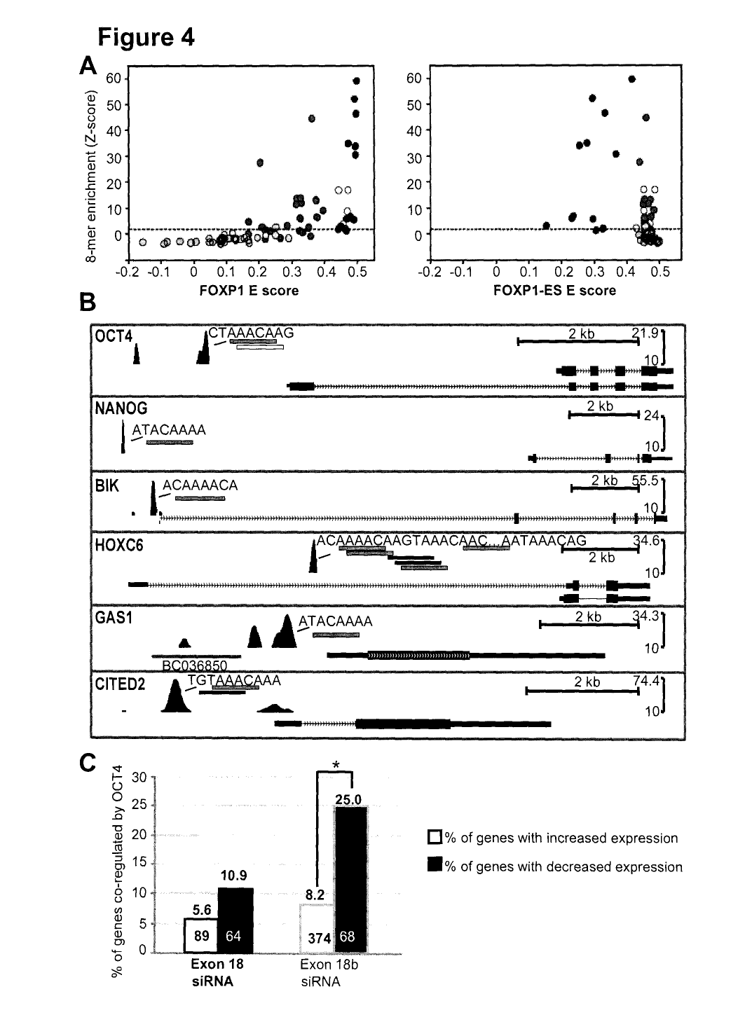

Scatterplots directly comparing the relative under-peak enrichment and PBM scores for the individual 8-mers are shown in FIG. 4A. Notably, PBM 8-mers that bind preferentially to FOXP1-ES or FOXP1, and other 8 mers that bind to both proteins are highly significantly enriched under the FOXP1-associated ChIP-Seq peaks. While a subset of the ChIP-Seq peaks contained sequences corresponding to the FOXP1-ES binding consensus CGATACA or closely related 8-mers, these sequences were not significantly enriched under the FOXP1 ChIP-Seq peaks.

These results show that FOXP1 and FOXP1-ES can preferentially bind to similar sequences in vivo as they do in vitro. However, the CGATACA consensus and closely related sequences preferentially bound by FOXP1-ES in vitro do not appear to be widely utilized by this factor in vivo. Instead, FOXP1-ES appears to more often bind a subset of C/A-rich motifs, and possibly lower affinity sites that resemble C/A-rich motifs also bound by FOXP1 (FIG. 4A). Previous studies (Jaeger et al., 2010; Rowan et al., 2010) have revealed examples of transcription factors that preferentially bind lower affinity sites in vivo, and it is possible that this may be important to facilitate the dynamic changes in transcriptional output mediated by FOXP1-ES and FOXP1 upon induction of ESC differentiation.

Examples of candidate direct target genes include several with functions in differentiation and early development and are shown in FIG. 4B. Importantly, the data support OCT4 and NANOG as possible direct targets of FOXP1-ES, since the promoters of these genes are proximal to peaks that overlap sequences predicted by the PBM analysis to bind preferentially to this isoform.

Further showing a direct role for FOXP1-ES in regulating OCT4, a statistically significant overlap was observed between the set of RNA-Seq profiled genes that are dependent on FOXP1-ES for expression in H9 hESCs (FIG. 3D), and a set of genes previously reported (Kunarso et al., 2010) to be both directly bound and regulated by OCT4 in H1 hESCs (FIG. 4C, p=0.0016; Chi-square test). The majority of these overlapping genes show changes in the same direction upon knockdown of either factor. In contrast, genes stimulated or repressed by FOXP1 in this hESC line, did not significantly overlap with the previously reported OCT4 target genes (FIG. 4C). Without wishing to be bound by theory, the results show that FOXP1-ES regulate hESC self-renewal and pluripotency maintenance by directly controlling the expression of OCT4. Moreover, the results also show that reduced expression of a subset of genes upon knockdown of FOXP1-ES in H9 hESCs arises as an indirect consequence of the loss of FOXP1-ES-dependent expression of OCT4.

Foxp1-ES Expression Promotes mESC Self-Renewal and Pluripotency

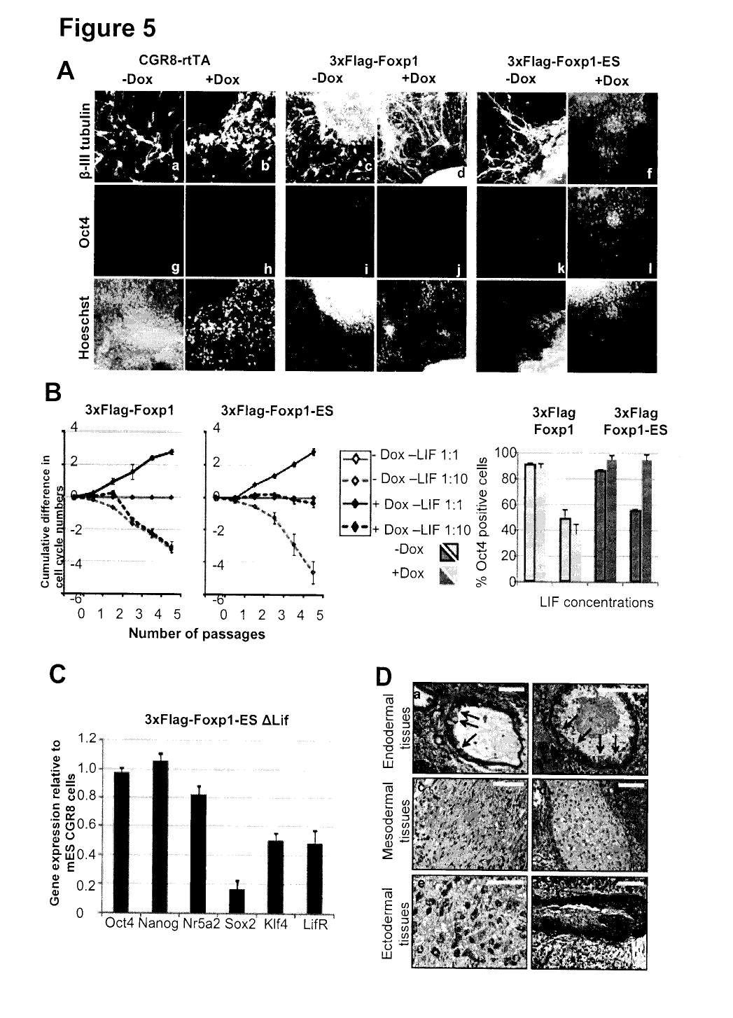

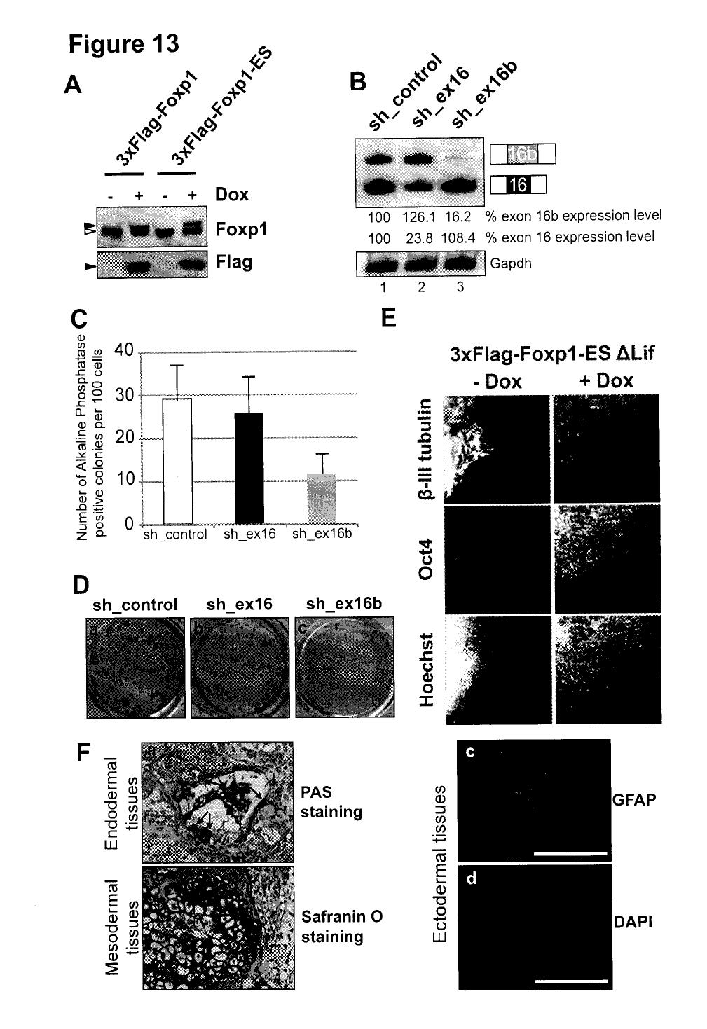

To show that Foxp1-ES is important for the maintenance of the self-renewal and pluripotency of mESCs, it was first asked whether increasing its expression over the endogenous levels of Foxp1 is capable of suppressing mESC differentiation. Transgenic CGR8 mESC lines were generated that stably express 3×Flag-Foxp1-ES or 3×Flag-Foxp1 isoforms under Doxycycline (Dox)-inducible control. Upon Dox stimulation 3×Flag-Foxp1 and 3×Flag-Foxp1-ES expression was detected at levels comparable to those of the endogenous proteins, thus resulting in an approximately 2-fold increase in the overall expression of each isoform (FIG. 13A). Cells from the parental (rtTA) and 3×Flag-Foxp1 isoform-expressing CGR8 lines were then aggregated to form embryoid bodies and cultured under conditions which favor neural cell differentiation. In the absence of Dox, all three cell lines supported neural differentiation, as revealed by the appearance of cells with neuronal morphology that immunostained with an antibody to the neuronal marker 13-III tubulin (FIG. 5Aa,c,e). Differentiated β-III tubulin-positive neurons were also observed in the control line and in 3×Flag-Foxp1-expressing cells after Dox stimulation (FIGS. 5Ab and d, respectively). In stark contrast, over-expression of 3×Flag-Foxp1-ES almost completely abolished neural cell differentiation as revealed by the absence of both neuronal-like cells and β-III tubulin immunostaining (FIG. 5Af). Oct4 expression in these cultures was also assessed to determine whether over-expression of Foxp1-ES but not of Foxp1 may promote the maintenance of CGR8 mESCs in an undifferentiated, self-renewing state (FIGS. 5Ag-I). Indeed, only the 3×Flag-Foxp1-ES over-expressing cells showed prominent levels of Oct4 immunostaining (compare FIG. 5Al with FIG. 5Ag-k). The effects of isoform-specific knockdown in CGR8 mESCs was also compared (FIG. 13B-D). Whereas knockdown of Foxp1 did not have significant impact on cell growth, knockdown of Foxp1-ES reduced formation of CGR8 mES colonies by ˜3-fold. Taken together, these results show that Foxp1 promotes mESC differentiation, whereas expression of Foxp1-ES prevents differentiation and is required for mESC self-renewal.

To further show that Foxp1-ES expression is required for the maintenance of stem cell identity, the Dox-inducible 3×Flag-Foxp1 or 3×Flag-Foxp1-ES CGR8 cell lines were cultured in the presence of different levels of Leukemia Inhibitory Factor (LIF), a cytokine which is required for pluripotency maintenance of mESCs. The CGR8 3×Flag-Foxp1 and 3×Flag-Foxp1-ES lines were cultured with an excess of LIF to support mESC self-renewal (FIG. 5B, LIF1:1, continuous lines), or with 10% of this amount, which is insufficient to prevent cell differentiation (FIG. 5B, LIF 1:10, dashed lines). Consistent with known characteristics of mESCs undergoing differentiation, in the absence of Dox induction, the reduced concentration of LIF led to reduced (˜50% of total) numbers of Oct4-positive cells after 4 cell passages (FIG. 5B, right panels) with reduced cell division rates (FIG. 5B, white dashed lines in left panels). However, upon Dox induction, over-expression of either Foxp1 isoform in the presence of standard LIF concentrations resulted in increased rates of cell division (FIG. 5B, solid black lines), and the majority (>80%) of cells remained Oct4 positive. In contrast, in the presence of reduced LIF, Dox-induction of 3×Flag-Foxp1 did not prevent cell differentiation; cell division declined after 2 passages and only ˜40% of the cells were Oct4 positive after 4 passages (FIG. 5B). Under the same reduced LIF conditions, Dox-induced expression of 3×Flag-Foxp1-ES prevented loss of pluripotency characteristics since more than 90% of the cells remained Oct4 positive after 4 passages and cell division rates were comparable to those of control cells grown in the presence of regular amounts of LIF (FIG. 5B).

To further show that overexpression of Foxp1-ES but not Foxp1 promotes the maintenance of pluripotency, the 3×Flag-Foxp1 and 3×Flag-Foxp1-ES expressing cell lines were cultured in the absence of exogenous LIF, with or without Dox added to the media. As expected, the two cell lines rapidly differentiated in the absence of Dox and the 3×Flag-Foxp1 line could not be maintained in culture beyond 5 or 6 passages even in the presence of Dox.Strikingly, the 3×Flag-Foxp1-ES line continued to grow in the presence of Dox and these cells were kept in culture over 30 passages in the absence of LIF. These cells are referred to as 3×Flag-Foxp1-ESΔLIF. qRT-PCR analysis performed after the 24th passage confirmed that these cells express Oct4, Nanog and Nr5a2 at levels comparable with those of the parental control CGR8 cells, but they display reduced levels of Sox2, Klf4 and LifR (FIG. 5C). The 3×Flag-Foxp1-ESΔLIF cells were aggregated to form embryoid bodies and then cultured under conditions that favor neural cell differentiation. As before, in the presence of Dox, the cells were predominantly immunopositive for Oct4 but not β-III tubulin (FIG. 13E, right panel). Conversely, in absence of Dox, the cells adopted clear neuronal morphology and stained positively for β-III tubulin, but displayed negligible immunostaining for Oct4 (FIG. 13E, left panel). Finally, when injected in mice, 3×Flag-Foxp1-ESΔLIF CGR8 mESCs are capable of forming teratomas which reproduce all three germ cell types in vivo (FIGS. 5D and 13F). Collectively, these results demonstrate that increased expression of Foxp1-ES, but not of Foxp1, promotes the maintenance of CGR8 mESCs in a pluripotent state.

### Foxp1-ES is Required for Efficient Reprogramming of Mouse Embryonic Fibroblasts to Induced Pluripotent Stem Cells

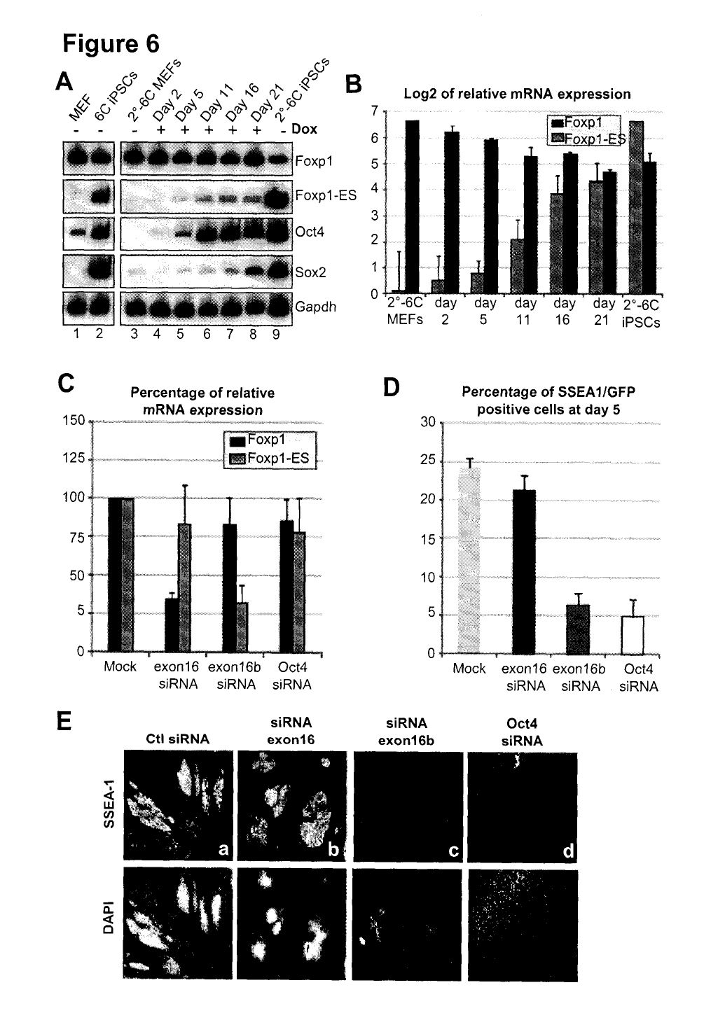

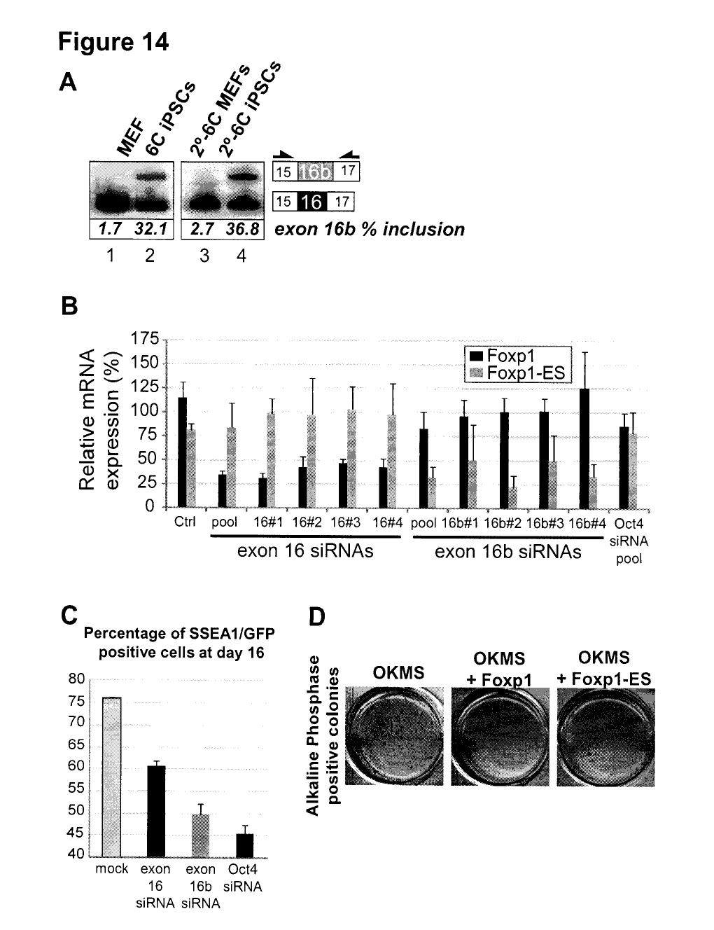

It was determined that Foxp1-ES expression is important for the formation of induced pluripotent stem cells (iPSCs) from mouse embryonic fibroblasts (MEFs). For this experiment, secondary mouse MEFs (2° MEFs) were employed that were derived from a primary iPSC line (1°-6C iPSC) containing an integrated piggyBac transposon expressing, under Dox-inducible control, the four Yamanaka transcription factors “OKMS” (Oct4, Klf4, c-Myc and Sox2) required for iPSC reprogramming (Takahashi and Yamanaka, 2006; Woltjen et al., 2009). In the presence of Dox, the 2°-6C MEFs expressing all four factors efficiently form secondary iPSCs (2°-6C iPSCs) that are pluripotent (Woltjen et al., 2009). Consistent with a key role for Foxp1-ES in the maintenance of mESC pluripotency, RT-PCR assays showed that exon 16b (homologous to human exon 18b) is almost completely skipped in primary MEFs but is included to ˜32% in Foxp1 transcripts in iPSCs, which is comparable with its inclusion level observed in mESCs (FIGS. 6A and 14A, lanes 1 and 2). During 2°-6C MEF reprogramming, Foxp1 exon 16 is predominantly included at early stages but displays progressively decreased inclusion towards the end of reprogramming (compare days 2-21 in FIGS. 6A, 6B and 14A). Conversely, exon 16b is weakly included (<4%) during the earliest stages of secondary iPSC reprogramming (lanes 3-5) but becomes more efficiently included at later stages (between days 5-16), reaching the highest level of inclusion (˜37%) in 2°-6C iPSCs (FIGS. 6A, 6B and 14A).

It was next investigated whether Foxp1 and Foxp1-ES are important for iPSC formation. Each isoform was selectively knocked down using siRNAs specific for either exon 16b or exon 16 sequences. For each knockdown, sets of four siRNAs targeted to each exon were initially used as pools. Short interfering RNAs comprising these pools that produced the most efficient isoform-specific knockdown (FIG. 14B) were then used in pairs to validate results. Each isoform-specific pool resulted in a selective reduction (>65%) in the expression of only its respective target mRNA isoform, relative to the samples that were mock-transfected or transfected with an siRNA to Oct4, which was included as a control to inhibit reprogramming (FIG. 6C). Short interfering RNAs were transfected into 2°-6C MEFs at day 0 or at day 13 during reprogramming, and then harvested 5 and 3 days later, respectively. For both time points, reprogramming colonies were either fixed and immunostained with antibody to SSEA-1, a marker of ESCs/iPSCs, or cells were collected and analyzed by flow-cytometry to quantify cells that are double-positive for SSEA-1 and GFP signal (expressed from the ROSA26 locus) in the entire reprogramming population.

Quantification of SSEA-1/GFP positive cells by flow cytometry at day 5 of reprogramming is shown in FIG. 6D, and representative panels of immunostained colonies are shown in FIG. 6E. As expected, in the absence of Dox-induced expression of OKMS, no SSEA-1/GFP-positive cells formed. In the presence of Dox, 2°-6C MEFs transfected with siRNAs targeting Oct4 transcripts reduced the population of SSEA-1/GFP positive cells by ˜5 fold compared to mock transfected cells (FIGS. 6D and E). Importantly, knockdown of Foxp1-ES resulted in a comparable (˜4-fold) reduction in SSEA-1/GFP positive cells, whereas knockdown of Foxp1 had little to no effect on this population (FIGS. 6D and E). Knockdown of Foxp1-ES (or Oct4) at Day 13, also significantly reduced the proportion of SSEA-1/GFP-positive cells (FIGS. S6C and D). Finally, it was also asked whether over-expression of Foxp1-ES and Foxp1, together with OKMS factors, differentially affects the reprogramming of primary MEFs. While over-expression of Foxp1-ES with OKMS factors did not substantially alter the efficiency of formation of SSEA-1-positive colonies, overexpression of Foxp1 completely blocked reprogramming by OKMS factors, since no AP or SSEA-1 positive colonies were detected (FIG. 14E).

Taken together with the results described earlier, these data show that the AS mediated switch controlling Foxp1-ES expression provides for efficient iPSC reprogramming as well as for the maintenance of ESC self-renewal and pluripotency.

### Example 2

### Identification of Factors that Regulate FOXP1 Exon 18b Alternative Splicing

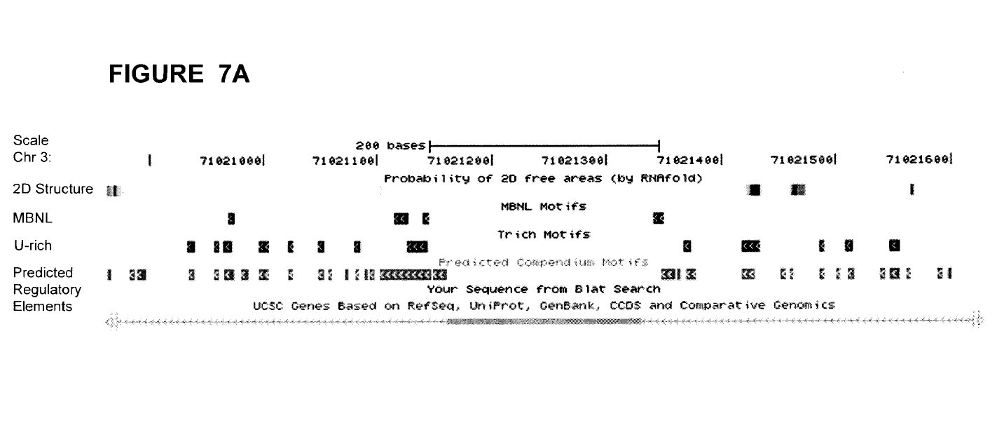

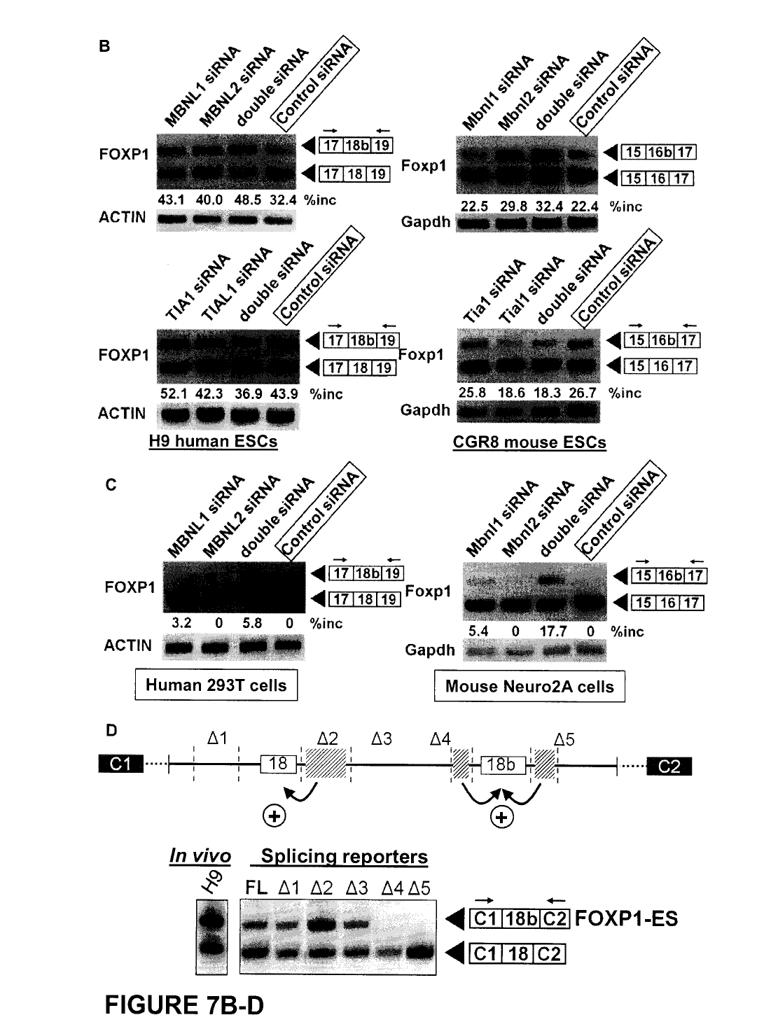

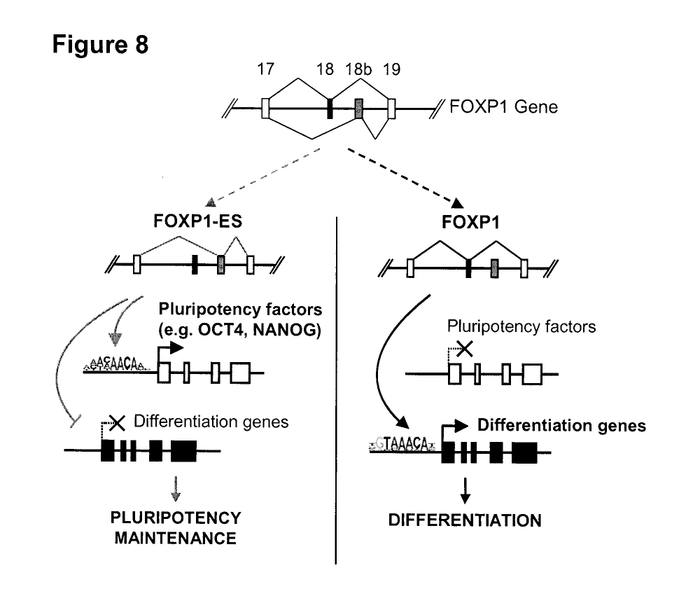

Using the recently reported splicing code (Barash et al. 2010) and manual inspection of intronic sequences located 300 nucleotides upstream and downstream of FOXP1 exon 18b, candidate cis-regulatory elements of this exon were identified. Both approaches identified sequences resembling binding sites of the regulators MBNL1-3 and TIA1/TIAL1 proteins, which recognize YGCUKY containing sequences- and U-rich motifs, respectively (FIG. 7A). In addition, the splicing code pinpointed several additional sequence elements predicted to function in exon 18b AS regulation, although at the present time the factor(s) recognizing these elements are not known (FIG. 7A).

Using transfection of Dharmacon siRNA pools, significant and reproducible modulation of exon 18b splicing following depletion of MBNL1, MBNL2, TIA1 and TIAL1 proteins was observed, but negligible effects on exon 18b splicing following knockdown of MBNL3, and two other tested splicing regulators, PTBP1 and PTBP2 (FIG. 7B). Knockdown of TIA1 and TIAL1 proteins resulted in decreased inclusion levels of exon 18b, whereas knockdown of MBNL1 and MBNL2 resulted in increased inclusion levels of exon 18b in human and mouse ESCs, with the largest changes seen when TIA1 and TIAL1 or MBNL1 and MBNL2 were knocked down simultaneously (FIG. 7B). This latter result suggested that MBNL1 and MBNL2 may act together to repress inclusion of FOXP1 exon 18b in non-ESCs/differentiated cells. To test this, each factor was knocked down independently and in combination in human 293T and mouse Neuro2A cell lines. In every case, the activation of exon 18b splicing in these cell lines was observed, with the strongest increase in inclusion when MBNL1 and MBNL2 were knocked down simultaneously (FIG. 7C). These results demonstrate a role for MBNL1, MBNL2, TIA1 and TIAL1 proteins in the regulation of FOXP1 exon 18b AS, with TIAL1 and TIAL1 functioning to stimulate exon 18b splicing, and MBNL1 and MBNL2 functioning to silence the inclusion of this exon in ESCs/iPSCs. These results show that the single and/or combined manipulation of the levels or activities of TIA1, TIAL1, MBNL1, MBNL2 or other regulators of FOXP1 exon 18b splicing have utility in applications directed at the maintenance of ESCs in a self-renewing and pluripotent state, as well as for the efficient and stable production of iPSCs.

In addition, MBNL1 and 2 affect the regulation of additional stem cell specific events. Without being bound by theory, these proteins may play a widespread role in negatively regulating a stem cell specific alternative splicing program. The suppression of this program by modulators such as MBNL1 and 2 is a factor in reprogramming somatic cells into pluripotent stem cells.

In order to delineate sequences that are critical for regulation of exon 18b splicing a pre-mRNA splicing reporter was generated by subcloning sequences of FOXP1 including the alternative exons 18 and 18b as well as flanking introns (˜1.1 kb) into the Exontrap vector (MoBiTec) (FIG. 7D). When transfected into H9 human ESCs, transcripts produced from this vector display patterns of splicing that are similar to those of endogenous FOXP1 pre-mRNA, with separate populations of transcripts that specifically include exon 18b or exon 18 (FIG. 7E). Importantly, deletion of 34 nucleotides of intronic sequence upstream of exon 18b or 52 nucleotides of intronic sequence downstream of exon 18b completely abolishes inclusion of exon 18b without affecting inclusion of exon 18 (FIG. 7D, lanes 6 and 7). These results demonstrate that critical regulatory elements required for exon 18b splicing reside in the deleted intronic regions flanking exon 18b, and are further consistent with the observation that binding sites for TIA1, TIAL1, MBNL1 and MBNL2, as well as additional sites predicted by the splicing code to be important for exon 18b splicing, are located in these intronic regions.

### Materials and Methods

### Microarray Hybridization, Data Extraction and Analysis

Total RNA was extracted from 1-2 g of sample of undifferentiated or differentiating H9 hESCs using TRI reagent (Sigma-Aldrich) as per the manufacturer recommendations. PolyA+ mRNA was purified using Nucleotrap Midiprep kits (Clonetech). Complementary DNA (cDNA) synthesized from the polyA+ RNA samples using the WT-Ovation RNA Amplification System (Nugen) was hybridized to custom AS microarrays in dye swap experiments and analyzed as described previously (Pan et al., 2004).

siRNA Knockdown of Splicing Regulators

Cells were transfected with SMART-pool siRNAs (Dharmacon) targeting potential splicing regulators using DharmaFECT reagent following the manufacturer's instructions, and total RNA was extracted 48 or 72 hours after transfection.

### RNA-Seq Data Generation and Analysis

H9 hESCs were transfected with a control, non-targeting siRNA pool (Dharmacon) or with siRNA pools targeting FOXP1 exon 18 or exon 18b sequences using DharmaFECT reagent following the manufacturer recommendations. Two days after transfection, H9 hESCs were transfected again and total RNA was isolated after an additional two days of culture. Total RNA from two independent transfections was pooled and submitted to IIlumina Inc. for polyA+ isolation and mRNA sequencing; datasets consisting of ˜50 million×50 mer reads were generated from each sample. RNA-Seq analysis was performed using a reference database comprising all annotated human splice junctions, essentially as previously described (Pan et al., 2008).

### Reverse-Transcription-Polymerase Chain Reaction (RT-PCR) Assays

Radiolabeled, semi-quantitative RT-PCR reactions contained 10 ng of total RNA were performed using the OneStep kit (Qiagen), essentially as described previously (Calarco et al., 2007). For qRT-PCR analysis, cDNAs were generated from 2 μg of total RNA using SuperScript® III Reverse Transcriptase (Invitrogen) as per manufacturer recommendations, and reactions were performed in a 384 well format using 20 ng of cDNA and FastStart Universal SYBR Green Master (Rox) (Roche Applied Science).

### Protein Binding Microarrays (PBMs) and Data Analysis

GST-FOXP1 and GST-FOXP1-ES were expressed, purified and incubated on PBMs as previously described (Badis et al., 2009). Each protein was assayed using two independent array designs, and the resulting data was processed as described in (Lam et al., 2011). Individual 8-mers obtaining an E score >0.45 in at least one of the two experimental repeats for FOXP1 and FOXP1-ES were considered significant (Berger et al., 2008) and aligned to generate consensus sequences using enoLOGOS (Workman et al., 2005).

### Gel Mobility Shift Assays

dsDNA probes for electrophoretic mobility shift assays (EMSAs) contained two tandemly repeated copies of representative PBM-derived FOXP1/FOXP1-ES consensus binding sequences, or mutated derivatives of these sequences, separated by two cytosines. EMSAs were performed as described in (Hellman and Fried, 2007).

### ChIP-Seq

ChIP-Seq experiments were performed as described previously (Schmidt et al., 2009), using an anti-FOXP1 (Abcam) antibody and 5×10⁷ H9 hESCs per sample. Immunoprecipitated DNA and a fraction of the total input DNA (after fragmentation) were used to generate dsDNA libraries, which were prepared using the Genomic DNA Sample preparation kit (Illumina, FC-102-1001) as per the manufacturer's protocol.

### Immunofluoresence Microscopy

CGR8 cells were fixed in 4% formaldehyde/PBS for 10 min at room temperature, washed with PBS, permeabilized for 3 min at 4° C. with 0.1% Triton X100, and then incubated with blocking solution (0.01% goat serum, 10 mg/ml BSA, 0.2% Tween-20) for one hour at 37° C. Cells were incubated with primary antibodies (polyclonal β-III Tubulin, T2200 Sigma-Aldrich and murine monoclonal Oct4 Pierce) in blocking solution (1% goat serum, 1% BSA, 0.2% Tween20 in PBS) for two hours at 37°, and then with secondary antibodies in blocking solution for two hours at 37° C. Cells were washed in PBS, incubated with Hoechst dye (Sigma-Aldrich, B2883), mounted with Aqueous mounting Medium (Permafluor #0725) and images were acquired by epi-fluorescence imaging as previously described (Samavarchi-Tehrani et al., 2010).

iPSC Reprogramming Assays and Imaging

2°-6C MEF cells were cultured and induced to express OKMS factors as previously described (Woltjen et al., 2009). Single or pooled siRNAs (Dharmacon/ThermoFisher) were transfected 0 or 13 days after induction of OKMS factors, and cells were cultured for another 5 or 3 days prior to harvesting for total RNA, or analysis by Flow-cytometry and immunostaining, which were performed as previously described (Samavarchi-Tehrani et al., 2010).

### Example 3

### Muscleblind-Like Proteins MBNL1 and MBNL2 are Negative Regulators of the ESC-Specific AS Switch in FOXP1

As discussed above, Muscleblind-like proteins MBNL1 and MBNL2 have been identified as negative regulators of an ESC/iPSC-specific AS switch in the forkhead family transcription factor FOXP1 that controls pluripotency. These proteins are expressed at lower levels in ESCs than in diverse differentiated cells and tissues, and their knockdown promotes ESC/iPSC-specific AS in differentiated cells. It is further shown that among a large network of ESC-specific AS events in genes with diverse functions associated with ESC biology, approximately half are regulated by MBNL proteins. Consistent with a central role for MBNL proteins in the core pluripotency circuitry, their knockdown promotes the expression of key pluripotency genes early on during the reprogramming of somatic cells to induced pluripotent stem cells.

To identify trans-acting regulators of ESC-specific AS events, high-throughput RNA sequencing (RNA-Seq) was used to detect human and mouse splicing factor genes that are differentially expressed between ESCs and a diverse panel of non-ESC lines and differentiated tissues. From the same RNA-Seq datasets, sets of human and mouse cassette alternative exons that display differential regulation between ESCs and non-ESCs and tissues were defined. A splicing code inference method (Barash et al. 2010; Xiong et al. 2011) was employed to predict cis-acting elements required for the regulation of these ESC/iPSC-specific AS events. By integrating results from these data, differentially expressed splicing regulators corresponding to defined binding sequences the code predicts to be important for controlling ESC-specific AS were identified.

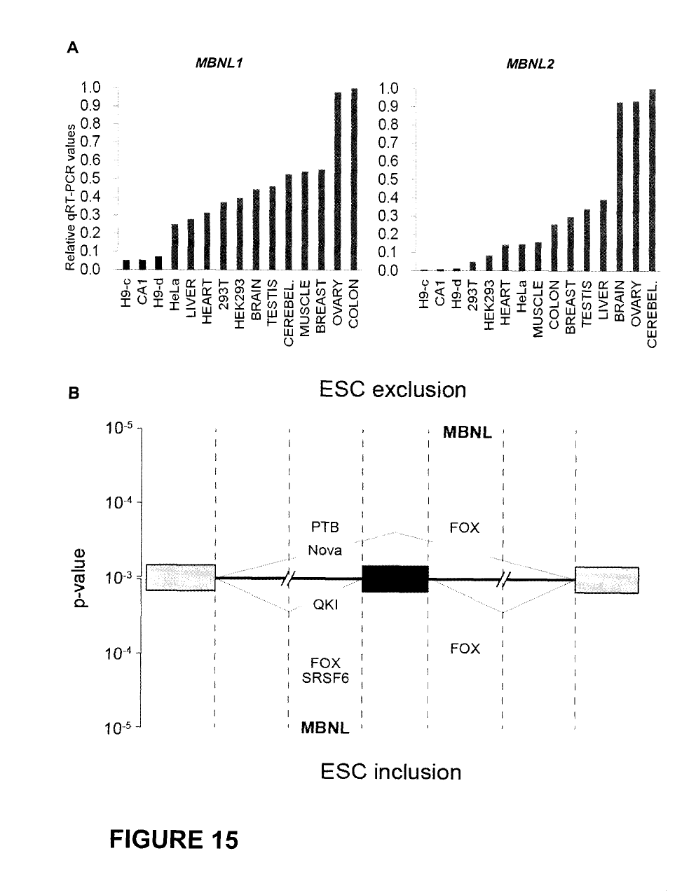

The mRNA expression levels of 221 known or putative splicing regulators across 7 human ESC/iPSC lines and 28 diverse other cell line and tissue samples were determined using unique-mapping RNA-Seq reads and the metric “corrected reads per kilobase cDNA per million reads” (cRPKM). Eleven of these 221 genes are significantly differentially-expressed between ESCs and the other cells and tissues (Bonferroni-corrected p<0.05, Wilcox test). MBNL1 and MBNL2 have the lowest overall mRNA expression levels in ESCs and iPSCs compared with almost all of the other analyzed cell lines and tissues. Quantitative RT-PCR assays confirmed these observations (FIG. 15a). Similar results were obtained when analyzing a comparable collection of mouse ESC and non-ESC lines and tissues.

From the RNA-Seq data 208 human and 104 mouse ESC-regulated cassette AS events were defined, with each set comprising comparable numbers of exons that are on average ≧25% more included or more skipped in ESCs versus the other profiled cell lines and tissues. Alternative splicing of ESC-regulated exons are more conserved between human and mouse than are non-regulated exons (58.7% vs. 23.5% in human, p<2.2e-16, Fisher test). When comparing exons in both species that meet minimal required expression criteria for comparing ESC and non-ESC percent spliced in (PSI) levels, 27 (13-26%) of the human and mouse ESC-specific AS events overlapped. In addition to detecting previously identified ESC-specific AS events such as FOXP1 exon 18b, examples of novel conserved ESC-specific AS events are in MTA1, a component of the NuRD chromatin remodeling complex that has previously been implicated in the control of pluripotency (Liang et al. 2008), and in the protein kinases CASK, MARK2 and MAP2K7. These results thus suggest a more extensive role for regulated AS events in fundamental aspects of ESC biology than previously appreciated.

Using the splicing code, it was inferred the combinations of cis-regulatory elements that are predictive of ESC-specific AS in human and mouse. Consistent with the analysis of differential expression of trans-acting factors, consensus binding sites for MBNL proteins are the most strongly predictive of exons with both higher or lower inclusion levels in ESCs relative to other cells and tissues (FIG. 15b). Features resembling binding sites for RbFox2 are also predictive of ESC-regulated exons, although to a much lesser extent than MBNL features (FIG. 15b). Collectively, the results suggest that MBNL proteins may have an important role in the regulation of mammalian ESC-specific AS.

Because MBNL1/Mbnl1 and MBNL2/Mbnl2 proteins are expressed at very low levels in human and mouse ESCs compared to other cell types and tissues, without being bound by theory, it is hypothesized that these factors may function by repressing the inclusion of ESC-specific exons in non-ESCs, and, conversely, that they may also function by activating the inclusion of exons in non-ESCs that are normally skipped in ESCs. Previous studies performed in differentiated cell lines and tissues have shown that MBNL proteins can suppress exon inclusion when they bind upstream flanking intronic sequences, whereas they can promote inclusion when binding to downstream flanking intron sequences. The results from the splicing code are entirely consistent with this proposal: predicted MBNL binding elements are significantly enriched in intronic sequence upstream of exons that are specifically included in ESCs, and they are significantly enriched in intronic sequence downstream of exons that are specifically skipped in ESCs (FIG. 15b).

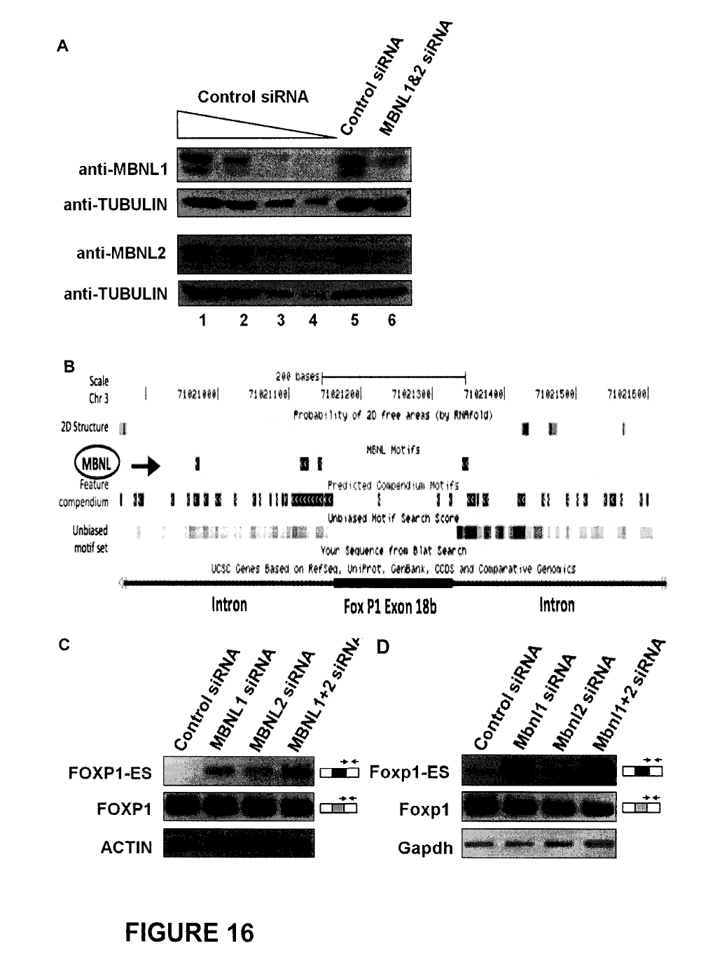

To directly test whether MBNL1/Mbnl1 and MBNL2/Mbnl2 proteins regulate ESC-specific AS events, siRNAs were transfected to knockdown these proteins individually and together in differentiated human and mouse cell lines and, for control and comparison purposes, in ESCs. RT-PCR assays were then used to monitor changes in the inclusion levels of ESC-specific AS events. Western blotting indicated that knockdown in all tested differentiated cells was efficient, resulting in less than 10% of the endogenous proteins remaining (FIG. 16a). The initial focus was on human FOXP1 exon 18b and the orthologous mouse Foxp1 exon 16b, which are specifically included in ESCs but skipped in differentiated cells. Moreover, the splicing code predicted that MBNL binding sites may mediate the repression of inclusion of these exons (FIG. 16b).

Knockdown of MBNL2 in 293T cells had no apparent effect on exon 18b inclusion. In contrast, knockdown of MBNL1 alone, and together with MBNL2, resulted in shifts from zero to 2.4 and 6.5 exon PSI, respectively (FIG. 16c). Similar but more pronounced effects were observed for mouse exon 16b: knockdown of Mbnl1 and Mbnl2 together in the neural cell line N2A resulted in a shift from no inclusion in the siRNA control transfection to 15.1 PSI, whereas knockdown of MBNL1 alone had a lesser effect (9.5 PSI) and knockdown of MBNL2 alone had no effect on exon 16b splicing (FIG. 16d). Knockdown in ESCs had little to no effect on exon 16b/18b splicing, consistent with the already low levels of expression of MBNL/Mbnl proteins in these cells. Collectively, the results are consistent with conserved, direct and partially redundant roles for MBNL1/Mbnl1 and MBNL2/Mbnl2 proteins in the negative regulation of FOXP1/Foxp1 exon 18b/16b splicing. Consistent with the splicing code results (FIG. 15b), while not being bound by theory, it is suggested that while these factors are important for FOXP1/Foxp1 exon 16b/18b regulation, they may not be sufficient for full regulation (see also below), although it cannot be excluded that incomplete knockdown might prevent levels of inclusion of these exons that are closer to those found in ESCs (˜60 PSI for exon 18b in H9 hESCs and 30-40 PSI for exon 16b in CGR8 mESCs; Gabut et al. 2011).

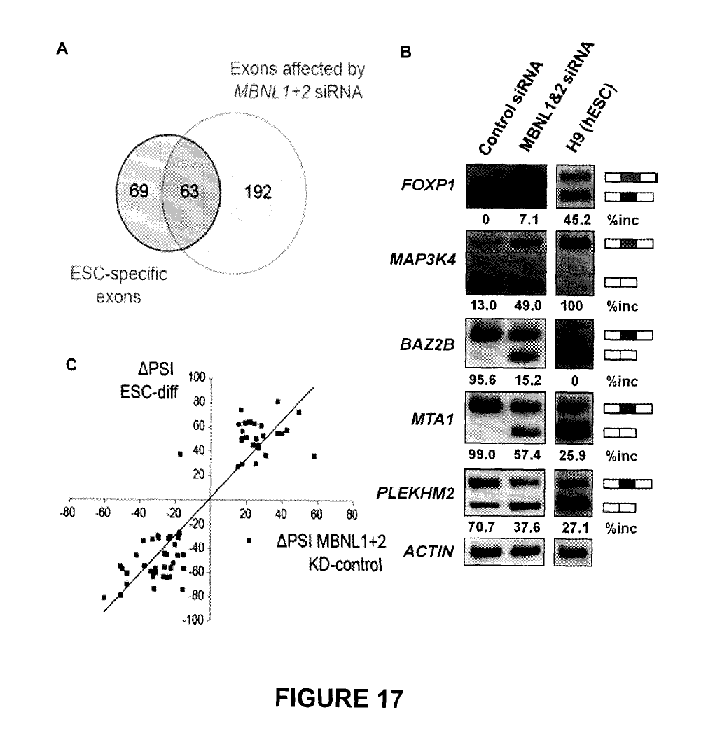

The extent to which ESC/iPSC-specific AS events are regulated by MBNL proteins was investigated next. MBNL1 and MBNL2 were simultaneously knocked down in HeLa cells, as described above, and RNA-Seq profiling was used to detect knockdown-dependent AS level changes (FIG. 17). Of 132 exons that have a 25 PSI difference between ESCs and differentiated cell lines and tissues and are expressed in HeLa cells, approximately half are also affected by knockdown of MBNL1/2 proteins, with a PSI change of in the expected direction (p<2.0e-49, hypergeometric test) (FIG. 17a). Moreover, a strong overall correlation (r=0.90) was observed when comparing the differences in PSI levels for exons differentially spliced between ESCs and non-ESCs/tissues with the differences in PSI levels of the same exons following knockdown of MBNL1/2 in HeLa cells (FIG. 17b). Comparable results were observed when MBNL1 and MBNL2 were knocked down in human 293T cells, and in undifferentiated C2C12 mouse myoblast cells. RT-PCR experiments validated the MBNL1+MBNL2 knockdown-dependent changes in PSI levels involving ESC-specific exons at high rate (˜90%; representative examples shown in FIG. 17c).

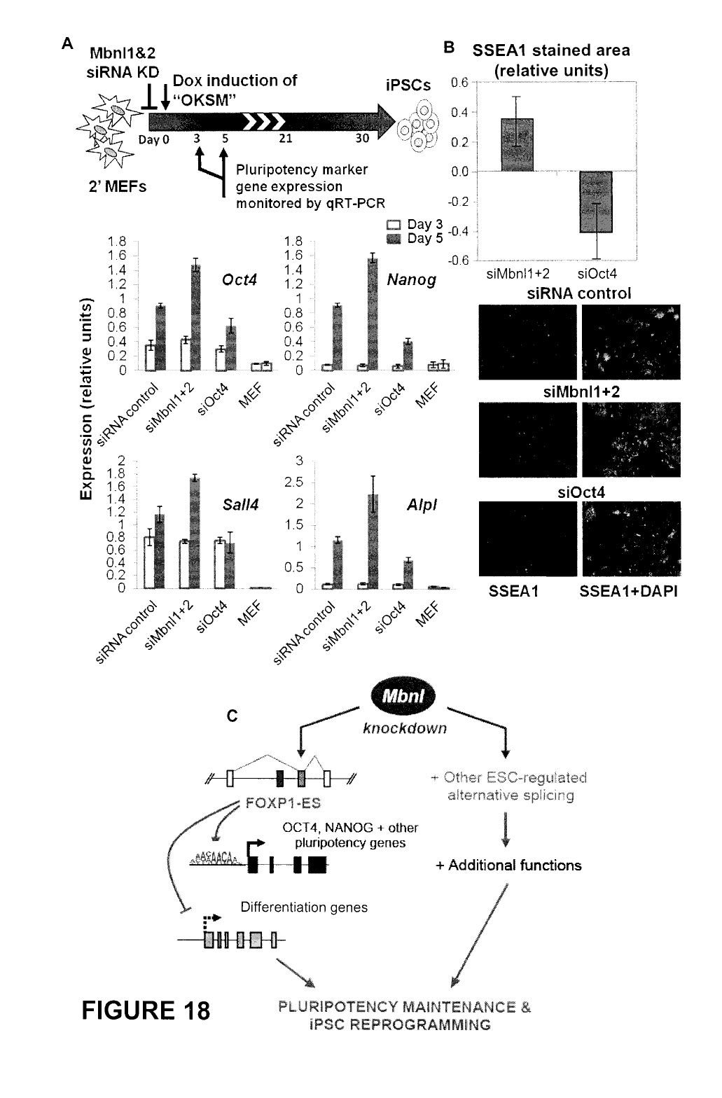

Given the important role for MBNL/Mbnl proteins in the regulation of FOXP1/Foxp1 exons 18b/16b, which control ESC pluripotency, it was next asked whether these splicing regulators control the expression of key pluripotency genes during the process of iPSC reprogramming. Secondary mouse embryonic fibroblasts (MEFs) expressing the four Yamanka factors “OKMS” (Oct4, Klf4, c-Myc, and Sox2) under doxycycline (dox)-inducible control were transfected with siRNA pools that knockdown Mbnl1 and Mbnl2, or with a control non-targeting siRNA pool. A siRNA pool targeting Oct4 was transfected as a negative control. The expression of several pluripotency factors and markers, including Oct4, Nanog, Sall4 and Alpl, were measured by qRT-PCR assays three and five days post induction of OKMS (FIG. 18a). At day 3 post-induction, none of these genes displayed significant changes in expression between the different knockdowns conditions. However, at day 5 post-induction, simultaneous knockdown of Mbnl1 and Mbnl2 stimulated the expression of Oct4, Nanog, Sall4 and Alpl by 1.5 to 2-fold over the levels observed in the control siRNA transfection (FIG. 18a). Knockdown of Mbnl1 and Mbnl2 also resulted in a statistically significant increase of 30% in the colony area immunostained with antibody to SSEA1, a marker for re-programming iPSCs (FIG. 18b). In contrast, as expected, knockdown of Oct4 resulted in a reduction in expression pluripotency genes, and also in SSEA1-immunostained colonies (FIGS. 18a,b). Thus, consistent with an important role for Mbnl proteins in suppressing the splicing of Foxp1 exon 16b, the inclusion of which is required for ESC pluripotency maintenance and the efficient iPSC programming (Gabut et al. 2011) knockdown of these factors significantly enhances the expression of key pluripotency genes at an early stage of reprogramming.

While the present disclosure has been described with reference to a number of examples, it is to be understood that the disclosure is not limited to the disclosed examples. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

TABLE OF SEQUENCES

| SEQ ID NO | Description |
|---|---|
| 1 | hFOXP1 (ex18) cDNA |
| 2 | exon 18 nucleic acid sequence |
| 3 | hFOXP1-ES (ex18b) cDNA |
| 4 | exon 18b nucleic acid sequence |
| 5 | mFoxP1 (ex16) cDNA |
| 6 | exon 16 nucleic acid sequence |
| 7 | mFoxP1-ES (ex16b) cDNA |
| 8 | exon 16b nucleic acid sequence |
| 9 | hFOXP1 (ex18) amino acid sequence |
| 10 | exon 18 amino acid sequence |
| 11 | hFOXP1-ES (ex18b) amino acid sequence |
| 12 | exon 18b amino acid sequence |
| 13 | mFoxp1 (ex16) amino acid sequence |
| 14 | exon 16 amino acid sequence |
| 15 | mFoxp1-ES (ex16b) amino acid sequence |
| 16 | exon 16b amino acid sequence |

Sequences of siRNAs for knockdown
of mouse and human FOXP1 isoforms

| | |
|---|---|
| 17 | siRNA targeting mouse Foxp1 |
| 18 | siRNA targeting mouse Foxp1 |
| 19 | siRNA targeting mouse Foxp1 |
| 20 | siRNA targeting mouse Foxp1 |
| 21 | siRNA targeting mouse Foxp1-ES |
| 22 | siRNA targeting mouse Foxp1-ES |
| 23 | siRNA targeting mouse Foxp1-ES |
| 24 | siRNA targeting mouse Foxp1-ES |
| 25 | siRNA targeting human FOXP1 |
| 26 | siRNA targeting human FOXP1 |
| 27 | siRNA targeting human FOXP1 |
| 28 | siRNA targeting human FOXP1 |
| 29 | siRNA targeting human FOXP1-ES |
| 30 | siRNA targeting human FOXP1-ES |
| 31 | siRNA targeting human FOXP1-ES |
| 32 | siRNA targeting human FOXP1-ES |

Sequences of shRNAs for knockdown
of mouse Foxp1 isoforms

| | |
|---|---|
| 33 | control shRNA directed against the GFP cDNA |
| 34 | shRNA directed against exon 16 |
| 35 | shRNA directed against exon 16b |

Genomic DNA sequences

| | |
|---|---|
| 36 | hFOXP1 genomic DNA exon 18 |
| | upstream flanking region |
| 37 | hFOXP1 genomic DNA exon 18 |
| | downstream flanking region |
| 38 | hFOXP1 genomic DNA exon 18b |
| | upstream flanking region |
| 39 | hFOXP1 genomic DNA exon 18b |
| | downstream flanking region |
| 40 | mFOXP1 genomic DNA Exon 16 |
| | upstream flanking region |
| 41 | mFOXP1 genomic DNA Exon 16 |
| | downstream flanking region |
| 42 | mFOXP1 genomic DNA Exon 16b |
| | upstream flanking region |
| 43 | mFOXP1 genomic DNA Exon 16b |
| | downstream flanking region |

### REFERENCES

Atlasi, Y., Mowla, S. J., Ziaee, S. A., Gokhale, P. J., and Andrews, P. W. (2008). OCT4 spliced variants are differentially expressed in human pluripotent and nonpluripotent cells. Stem Cells 26, 3068-3074.
Badis, G., Berger, M. F., Philippakis, A. A., Talukder, S., Gehrke, A. R., Jaeger, S. A., Chan, E. T., Metzler, G., Vedenko, A., Chen, X., et al. (2009). Diversity and complexity in DNA recognition by transcription factors. Science 324, 1720-1723.
Barash, Y., J. A. et al. (2010). “Deciphering the splicing code.” Nature 465(7294): 53-59.
Beissbarth, T., and Speed, T. P. (2004). GOstat: find statistically overrepresented Gene Ontologies within a group of genes. Bioinformatics 20, 1464-1465.
Berger, M. F., Badis, G., Gehrke, A. R., Talukder, S., Philippakis, A. A., Pena-Castillo, L., Alleyne, T. M., Mnaimneh, S., Botvinnik, O. B., Chan, E. T., et al. (2008). Variation in homeodomain DNA binding revealed by high-resolution analysis of sequence preferences. Cell 133, 1266-1276.
Berger, M. F., Philippakis, A. A., Qureshi, A. M., He, F. S., Estep, P. W., 3rd, and Bulyk, M. L. (2006). Compact, universal DNA microarrays to comprehensively determine transcription-factor binding site specificities. Nat Biotechnol 24, 1429-1435.
Brown, P. J., Ashe, S. L., Leich, E., Burek, C., Barrans, S., Fenton, J. A., Jack, A. S., Pulford, K., Rosenwald, A., and Banham, A. H. (2008). Potentially oncogenic B-cell activation-induced smaller isoforms of FOXP1 are highly expressed in the activated B cell-like subtype of DLBCL. Blood 111, 2816-2824.
Calarco, J. A., Xing, Y., Caceres, M., Calarco, J. P., Xiao, X., Pan, Q., Lee, C., Preuss, T. M., and Blencowe, B. J. (2007). Global analysis of alternative splicing differences between humans and chimpanzees. Genes Dev 21, 2963-2975.
Chen, X., Xu, H., Yuan, P., Fang, F., Huss, M., Vega, V. B., Wong, E., Orlov, Y. L., Zhang, W., Jiang, J., et al. (2008). Integration of external signaling pathways with the core transcriptional network in embryonic stem cells. Cell 133, 1106-1117.
Dasen, J. S., De Camilli, A., Wang, B., Tucker, P. W., and Jessell, T. M. (2008). Hox repertoires for motor neuron diversity and connectivity gated by a single accessory factor, FoxP1. Cell 134, 304-316.
Demir, E., and Dickson, B. J. (2005). fruitless splicing specifies male courtship behavior in Drosophila. Cell 121, 785-794.
Fic, W., Juge, F., Soret, J., and Tazi, J. (2007). Eye development under the control of SRp55/B52-mediated alternative splicing of eyeless. PLoS One 2, e253.
Forch, P., and Valcarcel, J. (2003). Splicing regulation in Drosophila sex determination. Prog Mol Subcell Biol 31, 127-151.
Gabut, M., Samavarchi-Tehrani, P., Wang, X., Slobodeniuc, V., O'Hanlon, D., Sung, H. K., Alvarez, M., Talukder, S., Pan, Q., Mazzoni, E. O., Nedelec, S., Wichterle, H., Woltjen, K., Hughes, T. R., Zandstra, P. W., Nagy, A., Wrana, J. L., Blencowe, B. J. (2011). An alternative splicing switch regulates embryonic stem cell pluripotency and reprogramming. Cell. 147, 132-46.
Hao, J., Daleo, M. A., Murphy, C. K., Yu, P. B., Ho, J. N., Hu, J., Peterson, R. T., Hatzopoulos, A. K., and Hong, C. C. (2008). Dorsomorphin, a selective small molecule inhibitor of BMP signaling, promotes cardiomyogenesis in embryonic stem cells. PLoS One 3, e2904.
Hellman, L. M., and Fried, M. G. (2007). Electrophoretic mobility shift assay (EMSA) for detecting protein-nucleic acid interactions. Nat Protoc 2, 1849-1861.
Hu, H., Wang, B., Borde, M., Nardone, J., Maika, S., Allred, L., Tucker, P. W., and Rao, A. (2006). Foxp1 is an essential transcriptional regulator of B cell development. Nat Immunol 7, 819-826.
Jaeger, S. A., Chan, E. T., Berger, M. F., Stottmann, R., Hughes, T. R., and Bulyk, M. L. (2010). Conservation and regulatory associations of a wide affinity range of mouse transcription factor binding sites. Genomics 95, 185-195.
Kim, J., Chu, J., Shen, X., Wang, J., and Orkin, S. H. (2008). An extended transcriptional network for pluripotency of embryonic stem cells. Cell 132, 1049-1061.
Koh, K. P., Sundrud, M. S., and Rao, A. (2009). Domain requirements and sequence specificity of DNA binding for the forkhead transcription factor FOXP3. PLoS One 4, e8109.
Konstantoulas, C. J., Parmar, M., and Li, M. (2010). FoxP1 promotes midbrain identity in embryonic stem cell-derived dopamine neurons by regulating Pitx3. J Neurochem 113, 836-847.
Koon, H. B., Ippolito, G. C., Banham, A. H., and Tucker, P. W. (2007). FOXP1: a potential therapeutic target in cancer. Expert opinion on therapeutic targets 11, 955-965.
Kubo, H., Shimizu, M., Taya, Y., Kawamoto, T., Michida, M., Kaneko, E., Igarashi, A., Nishimura, M., Segoshi, K., Shimazu, Y., et al. (2009). Identification of mesenchymal stem cell (MSC)-transcription factors by microarray and knockdown analyses, and signature molecule-marked MSC in bone marrow by immunohistochemistry. Genes Cells 14, 407-424.
Kunarso, G., Chia, N. Y., Jeyakani, J., Hwang, C., Lu, X., Chan, Y. S., Ng, H. H., and Bourque, G. (2010). Transposable elements have rewired the core regulatory network of human embryonic stem cells. Nat Genet 42, 631-634.
Kunarso, G., Wong, K. Y., Stanton, L. W., and Lipovich, L. (2008). Detailed characterization of the mouse embryonic stem cell transcriptome reveals novel genes and intergenic splicing associated with pluripotency. BMC Genomics 9, 155.
Lam, K. N., van Bakel, H., Cote, A. G., van der Ven, A., and Hughes, T. R. (2011). Sequence specificity is obtained from the majority of modular C2H2 zinc-finger arrays. Nucleic Acids Research in press.
Langmead, B., Trapnell, C., Pop, M., and Salzberg, S. L. (2009). Ultrafast and memory-efficient alignment of short DNA sequences to the human genome. Genome Biol 10, R25.
Lee, L. H., Peerani, R., Ungrin, M., Joshi, C., Kumacheva, E., and Zandstra, P. (2009). Micropatterning of human embryonic stem cells dissects the mesoderm and endoderm lineages. Stem Cell Res 2, 155-162.
Liang, J., Wan, M., Zhang, Y., Gu, P., Xin, H., Jung, S. Y., Qin, J., Wong, J., Cooney, A. J., Liu, D., Songyang, Z. (2008). Nanog and Oct4 associate with unique transcriptional repression complexes in embryonic stem cells. Nat Cell Biol. 10, 731-9.
Li, S., Weidenfeld, J., and Morrisey, E. E. (2004). Transcriptional and DNA binding activity of the Foxp1/2/4 family is modulated by heterotypic and homotypic protein interactions. Mol Cell Biol 24, 809-822.
Lillie, R. D., Fulmer, H. M. (1976). Histopathologic Technic and Practical Histochemistry, 4th ed. New York, N.Y.: McGraw-Hill 1976:657
Mallanna, S. K., Ormsbee, B. D., Iacovino, M., Gilmore, J. M., Cox, J. L., Kyba, M., Washburn, M. P., and Rizzino, A. (2010). Proteomic analysis of Sox2-associated proteins during early stages of mouse embryonic stem cell differentiation identifies Sox21 as a novel regulator of stem cell fate. Stem Cells 28, 1715-1727.
Mayshar, Y., Rom, E., Chumakov, I., Kronman, A., Yayon, A., and Benvenisty, N. (2008). Fibroblast growth factor 4 and its novel splice isoform have opposing effects on the maintenance of human embryonic stem cell self-renewal. Stem Cells 26, 767-774.
Mortazavi, A., Williams, B. A., McCue, K., Schaeffer, L., and Wold, B. (2008). Mapping and quantifying mammalian transcriptomes by RNA-Seq. Nat Methods 5, 621-628.
Pan, Q., Shai, O., Lee, L. J., Frey, B. J., and Blencowe, B. J. (2008). Deep surveying of alternative splicing complexity in the human transcriptome by high-throughput sequencing. Nat Genet 40, 1413-1415.
Pan, Q., Shai, O., Misquitta, C., Zhang, W., Saltzman, A. L., Mohammad, N., Babak, T., Siu, H., Hughes, T. R., Morris, Q. D., et al. (2004). Revealing global regulatory features of mammalian alternative splicing using a quantitative microarray platform. Mol Cell 16, 929-941.
Peerani, R., Rao, B. M., Bauwens, C., Yin, T., Wood, G. A., Nagy, A., Kumacheva, E., and Zandstra, P. W. (2007). Niche-mediated control of human embryonic stem cell self-renewal and differentiation. EMBO J 26, 4744-4755.
Pritsker, M., Doniger, T. T., Kramer, L. C., Westcot, S. E., and Lemischka, I. R. (2005). Diversification of stem cell molecular repertoire by alternative splicing. Proc Natl Acad Sci USA 102, 14290-14295.
Puceat, M. (2008). Protocols for cardiac differentiation of embryonic stem cells. Methods 45, 168-171.
Rao, D. S., O'Connell, R. M., Chaudhuri, A. A., Garcia-Flores, Y., Geiger, T. L., and Baltimore, D. (2010a). MicroRNA-34a perturbs B lymphocyte development by repressing the forkhead box transcription factor Foxp1. Immunity 33, 48-59.
Rao, S., Zhen, S., Roumiantsev, S., McDonald, L. T., Yuan, G. C., and Orkin, S. H. (2010b). Differential roles of Sall4 isoforms in embryonic stem cell pluripotency. Mol Cell Biol 30, 5364-5380.
Rousso, D. L., Gaber, Z. B., Wellik, D., Morrisey, E. E., and Novitch, B. G. (2008). Coordinated actions of the forkhead protein Foxp1 and Hox proteins in the columnar organization of spinal motor neurons. Neuron 59, 226-240.
Rowan, S., Siggers, T., Lachke, S. A., Yue, Y., Bulyk, M. L., and Maas, R. L. (2010). Precise temporal control of the eye regulatory gene Pax6 via enhancer-binding site affinity. Genes & development 24, 980-985.
Salomonis, N., Schlieve, C. R., Pereira, L., Wahlquist, C., Colas, A., Zambon, A. C., Vranizan, K., Spindler, M. J., Pico, A. R., Cline, M. S., et al. (2010). Alternative splicing regulates mouse embryonic stem cell pluripotency and differentiation. Proc Natl Acad Sci USA 107, 10514-10519.
Samavarchi-Tehrani, P., Golipour, A., David, L., Sung, H. K., Beyer, T. A., Datti, A., Woltjen, K., Nagy, A., and Wrana, J. L. (2010). Functional genomics reveals a BMP-driven mesenchymal-to-epithelial transition in the initiation of somatic cell reprogramming. Cell Stem Cell 7, 64-77.
Schmidt, D., Wilson, M. D., Spyrou, C., Brown, G. D., Hadfield, J., and Odom, D. T. (2009). ChIP-seq: using high-throughput sequencing to discover protein-DNA interactions. Methods 48, 240-248.
Valouev, A., Johnson, D. S., Sundquist, A., Medina, C., Anton, E., Batzoglou, S., Myers, R. M., and Sidow, A. (2008). Genome-wide analysis of transcription factor binding sites based on ChIP-Seq data. Nat Methods 5, 829-834.
Silva, J., Nichols, J., Theunissen, T. W., Guo, G., van Oosten, A. L., Barrandon, O., Wray, J., Yamanaka, S., Chambers, I., and Smith, A. (2009). Nanog is the gateway to the pluripotent ground state. Cell 138, 722-737.
Stroud, J. C., Wu, Y., Bates, D. L., Han, A., Nowick, K., Paabo, S., Tong, H., and Chen, L. (2006). Structure of the forkhead domain of FOXP2 bound to DNA. Structure 14, 159-166.
Takahashi, K., and Yamanaka, S. (2006). Induction of pluripotent stem cells from mouse embryonic and adult fibroblast cultures by defined factors. Cell 126, 663-676.
Valcarcel, J., Singh, R., and Green, M. R. (1995). Mechanisms of regulated Pre-mRNA splicing.
Wang, B., Lin, D., Li, C., and Tucker, P. (2003). Multiple domains define the expression and regulatory properties of Foxp1 forkhead transcriptional repressors. J Biol Chem 278, 24259-24268.
Wang, W., Lin, C., Lu, D., Ning, Z., Cox, T., Melvin, D., Wang, X., Bradley, A., and Liu, P. (2008). Chromosomal transposition of PiggyBac in mouse embryonic stem cells. Proc Natl Acad Sci USA 105, 9290-9295.
Wang, B., Weidenfeld, J., Lu, M. M., Maika, S., Kuziel, W. A., Morrisey, E. E., and Tucker, P. W. (2004). Foxp1 regulates cardiac outflow tract, endocardial cushion morphogenesis and myocyte proliferation and maturation. Development 131, 4477-4487.
Wang, E. T., Sandberg, R., Luo, S., Khrebtukova, I., Zhang, L., Mayr, C., Kingsmore, S. F., Schroth, G. P., and Burge, C. B. (2008). Alternative isoform regulation in human tissue transcriptomes. Nature 456, 470-476.
Watanabe, K., Kamiya, D., Nishiyama, A., Katayama, T., Nozaki, S., Kawasaki, H., Watanabe, Y., Mizuseki, K., and Sasai, Y. (2005). Directed differentiation of telencephalic precursors from embryonic stem cells. Nat Neurosci 8, 288-296.
Wichterle, H., Lieberam, I., Porter, J. A., and Jessell, T. M. (2002). Directed differentiation of embryonic stem cells into motor neurons. Cell 110, 385-397.
Wijchers, P. J., Burbach, J. P., and Smidt, M. P. (2006). In control of biology: of mice, men and Foxes. Biochem J 397, 233-246.
Woltjen, K., Michael, I. P., Mohseni, P., Desai, R., Mileikovsky, M., Hamalainen, R., Cowling, R., Wang, W., Liu, P., Gertsenstein, M., et al. (2009). piggyBac transposition reprograms fibroblasts to induced pluripotent stem cells. Nature 458, 766-770.
Workman, C. T., Yin, Y., Corcoran, D. L., Ideker, T., Stormo, G. D., and Benos, P. V. (2005). enoLOGOS: a versatile web tool for energy normalized sequence logos. Nucleic Acids Res 33, W389-392.
Wu, J. Q., Habegger, L., Noisa, P., Szekely, A., Qiu, C., Hutchison, S., Raha, D., Egholm, M., Lin, H., Weissman, S., et al. (2010). Dynamic transcriptomes during neural differentiation of human embryonic stem cells revealed by short, long, and paired-end sequencing. Proc Natl Acad Sci USA 107, 5254-5259.
Xiong, H. Y., Barash, Y., Frey, B. J. (2011). Bayesian prediction of tissue-regulated splicing using RNA sequence and cellular context. Bioinformatics. 27, 2554-6.
Yeo, G. W., Xu, X., Liang, T. Y., Muotri, A. R., Carson, C. T., Coufal, N. G., and Gage, F. H. (2007). Alternative splicing events identified in human embryonic stem cells and neural progenitors. PLoS Comput Biol 3, 1951-1967.
Zhang, Y., Li, S., Yuan, L., Tian, Y., Weidenfeld, J., Yang, J., Liu, F., Chokas, A. L., and Morrisey, E. E. (2010). Foxp1 coordinates cardiomyocyte proliferation through both cell-autonomous and nonautonomous mechanisms. Genes Dev 24, 1746-1757.

1-11. (canceled)

12: A method of maintaining a homogeneous population of pluripotent stems cells, suppressing stem cell differentiation or reprogramming somatic cells into pluripotent stems cells comprising:
(1)(a) transfecting the stem cells or somatic cells with a cDNA encoding FOXP1-ES,
(b) transfecting the stem cells or somatic cells with a mRNA encoding FOXP1-ES,
(c) expressing cDNA encoding FOXP1-ES in the stem cells or somatic cells,
(d) administering FOXP1-ES protein to a culture of the stem cells or somatic cells,
(e) administering a stimulator of exon 18b inclusion to the stem cells or somatic cells,
(f) administering antisense RNA or interfering RNA that decreases the expression of FOXP1 to the stem cells or somatic cells, or
(g) administering genomic derived FOXP1 to the stem cells or somatic cells and expressing the FOXP1-ES isoform in the stem cells or somatic cells; and
(2) culturing under conditions that allow maintenance of the pluripotent stem cells, suppression of the stem cell differentiation or reprogramming of the somatic cells into induced pluripotent stem cells.

13. (canceled)

14: A method of producing a population of differentiated cells comprising:
(a) transfecting stem cells with a cDNA encoding FOXP1,
(b) transfecting stem cells with a mDNA encoding FOXP1,
(c) administering FOXP1 protein to stem cells,
(d) inhibiting the expression of FOXP1-ES in stem cells,
(e) administering a repressor of exon 18b inclusion to cells,
(f) administering antisense RNA or interfering RNA that decreases the expression of FOXP1-ES to cells, or
(g) administering genomic derived FOXP1 to cells and expressing the FOXP1 isoform in the cells;
and culturing the cells.

15: A method of modulating the expression of FOXP1-ES in a cell comprising administering an exon 18b splicing modulator to the cell.

16: The method of claim 15, wherein the exon 18b splicing modulator is a stimulator of exon 18b inclusion.

17: The method of claim 16, wherein the stimulator of exon 18b inclusion is selected from the group consisting of: TIA1, TIAL1, a MBNL1 antagonist, a MBNL2 antagonist and antisense RNA or interfering RNA that decreases expression of FOXP1.

18: The method of claim 17, wherein the MBNL1 and/or MBNL2 antagonist is an antibody or peptide or nucleic-acid derived aptamer to MBNL1 or MBNL2, antisense RNA or small interfering RNA that decreases expression of MBNL1 and/or MBNL2, or a compound that inhibits the expression or function of MBNL1 and/or MBNL2.

19: The method of claim 15, wherein the modulator is a repressor of exon 18b inclusion.

20: The method of claim 19, wherein the repressor of exon 18b inclusion is selected from the group consisting of: MBNL1, MBNL2, a TIA1 antagonist, a TIAL2 antagonist and antisense RNA or interfering RNA that decreases expression of FOXP1-ES.

21: The method of claim 20, wherein the TIA1 or TIAL2 antagonist is an antibody or peptide or nucleic-acid derived aptamer to TIA1 or TIAL1, antisense RNA or small interfering RNA that decreases expression of TIA1 or TIAL1, or a compound that inhibits the expression or function of TIA1 or TIAL1.

22: The method of claim 17 for maintaining or enhancing pluripotency in a cell.

23-25. (canceled)

26: The method of claim 22, wherein maintaining or enhancing pluripotency comprises producing pluripotent stem cells, maintaining a homogeneous population of pluripotent stem cells, suppressing stem cell differentiation or reprogramming somatic cells into pluripotent stem cells.

27-32. (canceled)

33: The method of claim 12, wherein the FOXP1-ES comprises the amino acid sequence as shown in SEQ ID NO: 11, 12, 15 or 16 or is encoded by the nucleic acid sequence as shown in SEQ ID NO: 3, 4, 7 or 8.

34: The method of claim 12, wherein the FOXP1 comprises the amino acid sequence as shown in SEQ ID NO: 9, 10, 13 or 14 or is encoded by the nucleic acid sequence as shown in SEQ ID NO: 1, 2, 5 or 6.

35: The method of claim 12, wherein the stimulator of exon 18b inclusion is selected from: TIA1, TIAL1, a MBNL1 antagonist and a MBNL2 antagonist.

36: The method of claim 14, wherein the FOXP1-ES comprises the amino acid sequence as shown in SEQ ID NO: 11, 12, 15 or 16 or is encoded by the nucleic acid sequence as shown in SEQ ID NO: 3, 4, 7 or 8.

37: The method of claim 14, wherein the FOXP1 comprises the amino acid sequence as shown in SEQ ID NO: 9, 10, 13 or 14 or is encoded by the nucleic acid sequence as shown in SEQ ID NO: 1, 2, 5 or 6.

38: The method of claim 14, wherein the repressor of exon 18b inclusion is selected from: MBNL1, MBNL2, a TIA1 antagonist, or a TIAL2 antagonist.
